SEQUENCE LISTING (92 sequences)

SEQ ID NO: 1 - protein, 16 aa, Homo sapiens
CARQEDTAMVYYFDYW

SEQ ID NO: 2 - DNA, 60 bp, Homo sapiens
tactgtgcgagacaggaggatacagctatggtttactactttgactactggggccaggga

SEQ ID NO: 3 - protein, 7 aa, Homo sapiens
RFWSGPL

SEQ ID NO: 4 - protein, 10 aa, Homo sapiens
HGNEVGTMWL

SEQ ID NO: 5 - protein, 11 aa, Homo sapiens
KNEKRWIHFDF

SEQ ID NO: 6 - protein, 5 aa, Homo sapiens
RLGIP

SEQ ID NO: 7 - protein, 15 aa, Homo sapiens
SHLVEELYLVAGEEG

SEQ ID NO: 8 - protein, 13 aa, Homo sapiens
VVYMQAATMLWYS

SEQ ID NO: 9 - protein, 13 aa, Homo sapiens
CSLYAGSNNVVVF

SEQ ID NO: 10 - protein, 17 aa, Homo sapiens
CASSASGGGGSNYKLTF

SEQ ID NO: 11 - protein, 13 aa, Homo sapiens
CASSYPGTAEAFF

SEQ ID NO: 12 - DNA, 334 bp, Homo sapiens
  misc_feature (1)..(334): nucleotide sequence encoding IgL1 (light chain 1) of x-mAb
cagtctgtgctgacgcagcctccctccgcgtccgggtcttttggacagtcagtcaccatctcctgcactggaaccagcagtgacattggtacttataattatgtctcctggtaccaacagcacccaggcagggcccccaaactcatgatttctgacatcaataagcggccctcaggggtccctgatcgcttctctggctccaagtctggcaacacggcctccctgaccgtctctggactccaggctgatgatgaggctgattattattgtagtctatatgcaggcagcaacaatgttgtggtattcggcggagggaccaagctgaccgtcctag

SEQ ID NO: 13 - DNA, 333 bp, Homo sapiens
  misc_feature (1)..(333): nucleotide sequence encoding IgL2 (light chain 2) of x-mAb
aattttatgctgactcagcctccctccgcgtccgggtcttttggacagtcagtcaccatctcctgcactggaaccaacagtgacattggtacttataattatgtctcctggtaccaacagcacccaggcaaggcccccaaactcatgatttctgacatcaataagcggccctcaggggtccctgatcgcttctctggctccaagtctggcaacacggcctccctgaccgtctctgactccaggctgatgatgaggctgattattattgtagtctatatgcaggcagcaacaatgttgtggtattcggcggagggaccaagctgaccgtcctag

SEQ ID NO: 14 - DNA, 349 bp, Homo sapiens
  misc_feature (1)..(349): nucleotide sequence encoding TCR-alpha
ctcactatagggcgaattgggcccgacgtcgcatgctcccggccgccatggcggccgcgggaattcgattgaaacaagaatagaaggagatattgtatcctatggtacaaaaaataccctgctgaaggtcctacattcctgatatctataagttccattaaggataaaaatgaagatggaagattcactgttttcttaaacaaaagtgccaagcacctctctctgcacattgtgccctcccagcctggagactctgcagtgtacttctgtgcagcaagcgcgtcgggtggtggaggtagcaactataaactgacatttggaaaaggaactctcttaaccgtgaatccaa

SEQ ID NO: 15 - DNA, 337 bp, Homo sapiens
  misc_feature (1)..(337): nucleotide sequence encoding TCR-beta
actatagggcgaattgggcccgacgtcgcatgctcccggccgccatggcggccgcgggaattcgattgaaacaagaatagaaggagatattgtatgtcctggtatcgacaagacccaggcatggggctgaggctgattcattactcagttggtgctggtatcactgaccaaggagaagtccccaatggctacaatgtctccagatcaaccacagaggatttcccgctcaggctgctgtcggctgctccctcccagacatctgtgtacttctgtgccagcagttaccccgggacggctgaagctttctttggacaaggcaccagactcacagttgtag

SEQ ID NO: 16 - protein, 15 aa, Homo sapiens
SHLVEALYLVCGERG

SEQ ID NO: 17 - DNA, 20 bp, Artificial sequence
  x-Id forward primer 1
gctggagtggattgggagta

SEQ ID NO: 18 - DNA, 26 bp, Artificial sequence
  x-Id reverse primer 1
cccagtagtcaaagtagtaaaccata

SEQ ID NO: 19 - DNA, 27 bp, Artificial sequence
  x-Id reverse primer 2
tccctggccccagtagtcaaagtagta

SEQ ID NO: 20 - protein, 13 aa, Homo sapiens
CAGGHNYGIKSYW

SEQ ID NO: 21 - protein, 15 aa, Homo sapiens
CARQRFWSGPLFDYW

SEQ ID NO: 22 - protein, 13 aa, Homo sapiens
RQENFDTAMVYYF

SEQ ID NO: 23 - DNA, 19 bp, Artificial Sequence
  x-Id forward primer 2
ctgcgctgtctctggttac

SEQ ID NO: 24 - DNA, 28 bp, Artificial Sequence
  x-Id reverse primer 3
tccctggcccccagtagtcaaagtagta

SEQ ID NO: 25 - DNA, 48 bp, Homo sapiens
  misc_feature (1)..(48): nucleotide sequence encoding CDR3 (x-Id clonotype)
tgtgcgagacaggaggatacagctatggtttactactttgactactgg

SEQ ID NO: 26 - DNA, 926 bp, Artificial sequence
  nucleotide sequence encoding heavy chain of x-mAb (including vector sequence)
tatgtatcatacacatacgatttaggtgacactatagaataacatccactttgcctttctctccacaggtgtccactcccaggtccaactgcacctcggttctatcgattgaattccaccatgggatggtcatgtatcatcctttttctagtagcaactgcaaccggtgtacattcccaggtgcagctgcaggagtcgggcccaggactggtgaagccttcggagaccctgtccctcacctgcgctgtctctggctactccatcagcagtggttactactggggctggatccggcagcccccagggaaggggctggagtggattgggagtatctatcatagtgggagcacctactacaacccgtccctcaagagtcgagtcaccatatcagtagacacgtccaagaaccagttctccctgaagctgagctctgtgaccgccgcagacacggccgtgtattactgtgcgagacaggaggatacagctatggtttactactttgactactggggccagggaaccctggtcaccgtctcctcagcgtcgaccaagggcccatcggtcttccccctggcaccctcctccaagagcacctctgggggcacagcggccctgggctgcctggtcaaggactacttccccgaacctgtgacggtctcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaactcctggggggaccgtcagtcttcctcttccccccaaa

SEQ ID NO: 27 - DNA, 364 bp, Homo sapiens
  misc_feature (1)..(364): nucleotide sequence encoding heavy chain of x-mAb
caggtgcagctgcaggagtcgggcccaggactggtgaagccttcggagaccctgtccctcacctgcgctgtctctggctactccatcagcagtggttactactggggctggatccggcagcccccagggaaggggctggagtggattgggagtatctatcatagtgggagcacctactacaacccgtccctcaagagtcgagtcaccatatcagtagacacgtccaagaaccagttctccctgaagctgagctctgtgaccgccgcagacacggccgtgtattactgtgcgagacaggaggatacagctatggtttactactttgactactggggccagggaaccctggtcaccgtctcctcag

SEQ ID NO: 28 - protein, 121 aa, Homo sapiens
  MISC_FEATURE (1)..(121): amino acid sequence of heavy chain of x-mAb
QVQLQESGPGLVKPSETLSLTCAVSGYSISSGYYWGWIRQPPGKGLEWIGSIYHSGSTYYNPSLKSRVTISVDTSKNQFSLKLSSVTAADTAVYYCARQEDTAMVYYFDYWGQGTLVTVSS

SEQ ID NO: 29 - DNA, 581 bp, Artificial Sequence
  nucleotide sequence encoding IgL1 (light chain 1) of x-mAb (including vector sequence)
ttctctccaccaggtgtcccactcccaggtccaactgcacctcggttctatcgattgaattccaccatgggatggtcatgtatcatcctttttctagtagcaactgcaaccggttccaattctcagactgtggtgacccagttctcaggctgtggcgttcttgagccaattttatgctgctctgtgacctccctatgagctgagtcagcacagactgggcccaggaaccggttcttgggccaattttatgctgactcagcctccctccgcgtccgggtcttttggacagtcagtcaccatctcctgcactggaaccagcagtgacattggtacttataattatgtctcctggtaccaacagcacccaggcaaggcccccaaactcatgatttctgacatcaataagcggctctcaggggtccctgatcgcttctctggctccaagtctggcaacacggcctccctgaccgtctctggactccaggctgatgatgaggctgattattattgtagtctatatgcaggcagcaacaatgttgtggtattcggcggagggaccaagctgaccgtcctagtcagcc

SEQ ID NO: 30 - protein, 111 aa, Homo sapiens
  MISC_FEATURE (1)..(111): amino acid sequence of IgL1 (light chain 1) of x-mAb
QSVLTQPPSASGSFGQSVTISCTGTSSDIGTYNYVSWYQQHPGRAPKLMISDINKRPSGVPDRFSGSKSGNTASLTVSGLQADDEADYYCSLYAGSNNVVVFGGGTKLTVL

SEQ ID NO: 31 - DNA, 885 bp, Artificial Sequence
  nucleotide sequence encoding IgL2 (light chain 2) of x-mAb (including vector sequence)
caatttaaccctttatgtatcatacacatacgatttaggtgacactatagaataacatccactttgcctttctctccacaggtgtccactcccaggtccaactgcacctcggttctatcgattgaattccaccatgggatggtcatgtatcatcctttttctagtagcaactgcaaccggttcttgggccaattttatgctgactcagcctccctccgcgtccgggtcttttggacagtcagtcaccatctcctgcactggaaccaacagtgacattggtacttataattatgtctcctggtaccaacagcacccaggcaaggcccccaaactcatgatttctgacatcaataagcggccctcaggggtccctgatcgcttctctggctccaagtctggcaacacggcctccctgaccgtctctgactccaggctgatgatgaggctgattattattgtagtctatatgcaggcagcaacaatgttgtggtattcggcggagggaccaagctgaccgtcctaggtcagcccagggctgccccctcggtcactctgttcccgccctcgagtgaggagcttcaagccaacaaggccacactggtgtgtctcataagtgacttctacccgggagccgtgacagtggcctggaaggcagatagcagccccgtcaaggcgggagtggagaccaccacaccctcccaaacaaagcaacaacaagtacgcggccagcagctatctgagcctgacgcctgagcagtggaagtcccacagaagctacagctgccaggtcacgcatgaagggagcaccgtggagaagacagtggcccctacagaatgttcatagaagcttggccgccatggcccaaacttgtttattgcagcctt

SEQ ID NO: 32 - protein, 109 aa, Homo sapiens
  MISC_FEATURE (1)..(109): amino acid sequence of IgL2 (light chain 2) of x-mAb
NFMLTQPPSASGSFGQSVTISCTGTNSDIGTYNYVSWYQQHPGKAPKLMISDINKRPSGVPDRFSGSKSGNTASLTVSDSRLMMRLIIIVVYMQAATMLWYSAEGPSPS

SEQ ID NO: 33 - protein, 115 aa, Homo sapiens
  MISC_FEATURE (1)..(115): amino acid sequence of TCR-alpha
LTIGRIGPDVACSRPPWRPREFDNKNRRRYCILWYKKYPAEGPTFLISISSIKDKNEDGRFTVFLNKSAKHLSLHIVPSQPGDSAVYFCAASASGGGGSNYKLTFGKGTLLTVNP

SEQ ID NO: 34 - protein, 111 aa, Homo sapiens
  MISC_FEATURE (1)..(111): amino acid sequence of TCR-beta
TIGRIGPDVACSRPPWRPREFDNKNRRRYCMSWYRQDPGMGLRLIHYSVGAGITDQGEVPNGYNVSRSTTEDFPLRLLSAAPSQTSVYFCASSYPGTAEAFFGQGTRLTVV

SEQ ID NO: 35 - DNA, 349 bp, Homo sapiens
  misc_feature (1)..(349): nucleotide sequence encoding TCR-alpha
gaccagcaagttaagcaaaattcaccatccctgagcgtccaggaaggaagaatttctattctgaactgtgactatactaacagcatgtttgattatttcctatggtacaaaaaataccctgctgaaggtcctacattcctgatatctataagttccattaaggataaaaatgaagatggaagattcactgtcttcttaaacaaaagtgccaagcacctctctctgcacattgtgccctcccagcctggagactctgcagtgtacttctgtgcagcaagcgcgtcgggtggtggaggtagcaactataaactgacatttggaaaaggaactctcttaaccgtgaatccaa

SEQ ID NO: 36 - DNA, 244 bp, Homo sapiens
  misc_feature (1)..(244): nucleotide sequence encoding TCR-beta
atgtcctggtatcgacaagacccaggcatggggctgaggctgattcattactcagttggtgctggtatcactgaccaaggagaagtccccaatggctacaatgtctccagatcaaccacagaggatttcccgctcaggctgctgtcggctgctccctcccagacatctgtgtacttctgtgccagcagttacctcgggacggctgaagctttctttggacaaggcaccagactcacagttgtag

SEQ ID NO: 37 - DNA, 27 bp, Homo sapiens
  misc_feature (1)..(27): nucleotide sequence encoding IGL1 CDR1
agcagtgacattggtacttataattat

SEQ ID NO: 38 - protein, 9 aa, Homo sapiens
  MISC_FEATURE (1)..(9): amino acid sequence of IGL1 CDR1
SSDIGTYNY

SEQ ID NO: 39 - DNA, 9 bp, Homo sapiens
  misc_feature (1)..(9): nucleotide sequence encoding IGL1 CDR2
gacatcaat

SEQ ID NO: 40 - protein, 3 aa, Homo sapiens
  MISC_FEATURE (1)..(3): amino acid sequence of IGL1 CDR2
DIN

SEQ ID NO: 41 - DNA, 36 bp, Homo sapiens
  misc_feature (1)..(36): nucleotide sequence encoding IGL1 CDR3
tgtagtctatatgcaggcagcaacaatgttgtggta

SEQ ID NO: 42 - protein, 12 aa, Homo sapiens
  MISC_FEATURE (1)..(12): amino acid sequence of IGL1 CDR3
CSLYAGSNNVVV

SEQ ID NO: 43 - DNA, 26 bp, Homo sapiens
  misc_feature (1)..(26): nucleotide sequence encoding IGH CDR1
ggctactccatcagcagtggttacta

SEQ ID NO: 44 - protein, 9 aa, Homo sapiens
  MISC_FEATURE (1)..(9): amino acid of IGH CDR1
GYSISSGYY

SEQ ID NO: 45 - DNA, 21 bp, Homo sapiens
  misc_feature (1)..(21): nucleotide sequence encoding IGH CDR2
atctatcatagtgggagcacc

SEQ ID NO: 46 - protein, 7 aa, Homo sapiens
  MISC_FEATURE (1)..(7): amino acid sequence of IGH CDR2
IYHSGST

SEQ ID NO: 47 - DNA, 18 bp, Homo sapiens
  misc_feature (1)..(18): nucleotide sequence encoding TCR-alpha CDR1
aacagcatgtttgattat

SEQ ID NO: 48 - protein, 6 aa, Homo sapiens
  MISC_FEATURE (1)..(6): amino acid sequence of TCR-alpha CDR1
NSMFDY

SEQ ID NO: 49 - DNA, 21 bp, Homo sapiens
  misc_feature (1)..(21): nucleotide sequence of TCR-alpha CDR2
ataagttccattaaggataaa

SEQ ID NO: 50 - protein, 7 aa, Homo sapiens
  MISC_FEATURE (1)..(7): amino acid sequence of TCR-alpha CDR2
ISSIKDK

SEQ ID NO: 51 - DNA, 51 bp, Homo sapiens
  misc_feature (1)..(51): nucleotide sequence encoding TCR-alpha CDR3
tgtgcagcaagcgcgtcgggtggtggaggtagcaactataaactgacattt

SEQ ID NO: 52 - protein, 17 aa, Homo sapiens
  MISC_FEATURE (1)..(17): amino acid sequence of TCR-alpha CDR3
CAASASGGGGSNYKLTF

SEQ ID NO: 53 - DNA, 15 bp, Homo sapiens
  misc_feature (1)..(15): nucleotide sequence encoding TCR-beta CDR1
atgaaccatgaatac

SEQ ID NO: 54 - protein, 5 aa, Homo sapiens
  MISC_FEATURE (1)..(5): amino acid sequence of TCR-beta CDR1
MNHEY

SEQ ID NO: 55 - DNA, 18 bp, Homo sapiens
  misc_feature (1)..(18): nucleotide sequence encoding TCR-beta CDR2
tcagttggtgctggtatc

SEQ ID NO: 56 - protein, 6 aa, Homo sapiens
  MISC_FEATURE (1)..(6): amino acid sequence of TCR-beta CDR2
SVGAGI

SEQ ID NO: 57 - DNA, 48 bp, Homo sapiens
  misc_feature (1)..(48): nucleotide sequence encoding TCR-beta CDR3
gccagcagttacctcgggacggctgaagctttctttggacaaggcacc

SEQ ID NO: 58 - protein, 13 aa, Homo sapiens
  MISC_FEATURE (1)..(13): amino acid sequence of TCR-beta CDR3
CASSYLGTAEAFF

SEQ ID NO: 59 - DNA, 24 bp, Mus musculus
  misc_feature (1)..(24): nucleotide sequence encoding IGH CDR1 of anti-idiotypic Ab
ggttacaggtttaccagatatggg

SEQ ID NO: 60 - protein, 8 aa, Mus musculus
  MISC_FEATURE (1)..(8): amino acid sequence of IGH CDR1 of anti-idiotypic Ab
GYRFTRYG

SEQ ID NO: 61 - DNA, 24 bp, Mus musculus
  misc_feature (1)..(24): nucleotide sequence encoding IGH CDR2 of anti-idiotypic Ab
atcagcgcatacagtggagacaca

SEQ ID NO: 62 - protein, 8 aa, Mus musculus
  MISC_FEATURE (1)..(8): amino acid sequence of IGH CDR2 of anti-idiotypic Ab
ISAYSGDT

SEQ ID NO: 63 - DNA, 57 bp, Mus musculus
  misc_feature (1)..(57): nucleotide sequence encoding IGH CDR3 of anti-idiotypic Ab
tgtgcgagagatcacgtccaaggggaagtgagcatatattattatgccatggacgtc

SEQ ID NO: 64 - protein, 19 aa, Mus musculus
  MISC_FEATURE (1)..(19): amino acid sequence of IGH CDR3 of anti-idiotypic Ab
CARDHVQGEVSIYYYAMDV

SEQ ID NO: 65 - DNA, 27 bp, Mus musculus
  misc_feature (1)..(27): nucleotide sequence encoding IGL CDR1 of anti-idiotypic Ab
agcagtgacattggtacttataattat

SEQ ID NO: 66 - protein, 9 aa, Mus musculus
  MISC_FEATURE (1)..(9): amino acid sequence of IGL CDR1 of anti-idiotypic Ab
SSDIGTYNY

SEQ ID NO: 67 - DNA, 9 bp, Mus musculus
  misc_feature (1)..(9): nucleotide sequence encoding IGL CDR2 of anti-idiotypic Ab
gacatcaat

SEQ ID NO: 68 - protein, 3 aa, Mus musculus
  MISC_FEATURE (1)..(3): amino acid sequence of IGL CDR2 of anti-idiotypic Ab
DIN

SEQ ID NO: 69 - DNA, 33 bp, Mus musculus
  misc_feature (1)..(33): nucleotide sequence encoding IGL CDR3 of anti-idiotypic Ab
agtctatatgcaggcagcaacaatgttgtggta

SEQ ID NO: 70 - protein, 11 aa, Mus musculus
  MISC_FEATURE (1)..(11): amino acid sequence of IGL CDR3 of anti-idiotypic Ab
SLYAGSNNVVV

SEQ ID NO: 71 - DNA, 36 bp, Mus musculus; identical to SEQ ID NO: 41
  misc_feature (1)..(36): nucleotide sequence of IGL CDR3 of anti-idiotypic Ab

SEQ ID NO: 72 - protein, 12 aa, Mus musculus
  MISC_FEATURE (1)..(12): amino acid sequence of IGL CDR3 of anti-idiotypic Ab
CSLYAGSNNVVV

SEQ ID NO: 73 - DNA, 376 bp, Mus musculus
  misc_feature (1)..(376): nucleotide sequence encoding IGH of anti-idiotypic Ab
gaggtgcagctggtgcagtctggacctgagatgaagaagcctggggcctcagtgaaggtctcctgcaagacttctggttacaggtttaccagatatgggatcagttgggtgcggcaggcccctggacgggggctggagtggctggggtggatcagcgcatacagtggagacacatattatggacagaaattccaggacagagtcaccatgactacagacagagccacgagtacagcctatatggagttgcggaacctgggatctgacgactcggccgtttatttctgtgcgagagatcacgtccaaggggaagtgagcatatattattatgccatggacgtctggggcgaagggaccacggtcaccgtctcctcag

SEQ ID NO: 74 - protein, 125 aa, Mus musculus
  MISC_FEATURE (1)..(125): amino acid sequence of IGH of anti-idiotypic Ab
EVQLVQSGPEMKKPGASVKVSCKTSGYRFTRYGISWVRQAPGRGLEWLGWISAYSGDTYYGQKFQDRVTMTTDRATSTAYMELRNLGSDDSAVYFCARDHVQGEVSIYYYAMDVWGEGTTVTVSS

SEQ ID NO: 75 - DNA, 334 bp, Mus musculus
  misc_feature (1)..(334): nucleotide sequence encoding IGL of anti-idiotypic Ab
cagcttgtgctgactcagcctccctccgcgtccgggtcttttggacagtcagtcaccatctcctgcactggaaccagcagtgacattggtacttataattatatctcctggtaccaacagcacccaggcaaggcccccaaactcatgatttctgacatcaataagcggccctcaggggtccctgatcgcttctctggctccaagtctggcaacacggcctccctgaccgtctctggactccaggctgatgatgaggctgattattattgtagtctatatgcaggcagcaacaatgttgtggtattcggcggagggaccaagctgaccgtcctag

SEQ ID NO: 76 - protein, 111 aa, Mus musculus
  MISC_FEATURE (1)..(111): amino acid sequence of IGL of anti-idiotypic Ab
QLVLTQPPSASGSFGQSVTISCTGTSSDIGTYNYISWYQQHPGKAPKLMISDINKRPSGVPDRFSGSKSGNTASLTVSGLQADDEADYYCSLYAGSNNVVVFGGGTKLTVL

SEQ ID NO: 77 - DNA, 24 bp, Mus musculus
  misc_feature (1)..(24): nucleotide sequence encoding IGH CDR1 of anti-idiotypic Ab
ggattcagcgtcagtgacaactac

SEQ ID NO: 78 - protein, 8 aa, Mus musculus
  MISC_FEATURE (1)..(8): amino acid sequence of IGH CDR1 of anti-idiotypic Ab
GFSVSDNY

SEQ ID NO: 79 - DNA, 21 bp, Mus musculus
  misc_feature (1)..(21): nucleotide sequence encoding IGH CDR2 of anti-idiotypic Ab
atttatagcggtggtagcaca

SEQ ID NO: 80 - protein, 7 aa, Mus musculus
  MISC_FEATURE (1)..(7): amino acid sequence of IGH CDR2 of anti-idiotypic Ab
IYSGGST

SEQ ID NO: 81 - DNA, 39 bp, Mus musculus
  misc_feature (1)..(39): nucleotide sequence encoding IGH CDR3 of anti-idiotypic Ab
tgtgcgggtggacacaactatggtataaagtcctactgg

SEQ ID NO: 82 - protein, 13 aa, Mus musculus
  MISC_FEATURE (1)..(13): amino acid sequence of IGH CDR3 of anti-idiotypic Ab
CAGGHNYGIKSYW

SEQ ID NO: 83 - DNA, 18 bp, Mus musculus
  misc_feature (1)..(18): nucleotide sequence encoding IGL CDR1 of anti-idiotypic Ab
cacgatattaacgacaac

SEQ ID NO: 84 - protein, 6 aa, Mus musculus
  MISC_FEATURE (1)..(6): amino acid sequence encoding IGL CDR1 of anti-idiotypic Ab
HDINDN

SEQ ID NO: 85 - DNA, 9 bp, Mus musculus
  misc_feature (1)..(9): nucleotide sequence encoding IGL CDR2 of anti-idiotypic Ab
ggtgcatcc

SEQ ID NO: 86 - protein, 3 aa, Mus musculus
  MISC_FEATURE (1)..(3): amino acid sequence encoding IGL CDR2 of anti-idiotypic Ab
GAS

SEQ ID NO: 87 - DNA, 36 bp, Mus musculus
  misc_feature (1)..(36): nucleotide sequence encoding IGL CDR3 of anti-idiotypic Ab
tgtcagcagtattataggtggcctccgctcactttc

SEQ ID NO: 88 - protein, 12 aa, Mus musculus
  MISC_FEATURE (1)..(12): amino acid sequence encoding IGL CDR3 of anti-idiotypic Ab
CQQYYRWPPLTF

SEQ ID NO: 89 - DNA, 352 bp, Mus musculus
  misc_feature (1)..(352): nucleotide sequence encoding IGH of anti-idiotypic Ab
gaggtgcagctgcaggagtctgggggaggcttggtccagccgggggggtccctgagactctcctgtgcagcctctggattcagcgtcagtaacaactacatgagttgggtccgccaggctccagggaaggggctggagtgggtctcagctatttatagcggtggtagcacatactacgcagactccgtgaagggccgattcaccatctccagagacaattccaagaacacgctgtttcttcaagtcaacagcctgggagctgaggacacggctgtctattactgtgcgggtggacacaactatggtataaagtcctactggggccagggaaccctggtcaccgtctcctcag

SEQ ID NO: 90 - protein, 117 aa, Mus musculus
  MISC_FEATURE (1)..(117): amino acid sequence of IGH of anti-idiotypic Ab
EVQLQESGGGLVQPGGSLRLSCAASGFSVSNNYMSWVRQAPGKGLEWVSAIYSGGSTYYADSVKGRFTISRDNSKNTLFLQVNSLGAEDTAVYYCAGGHNYGIKSYWGQGTLVTVSS

SEQ ID NO: 91 - DNA, 325 bp, Mus musculus
  misc_feature (1)..(325): nucleotide sequence encoding IGL of anti-idiotypic Ab
gaaatagtgatgacgcagtctccagccaccctatctttgtctccaggtgaaacggccaccctctcctgcagggccagtcacgatattaacgacaacttagcctggtaccagcagaaacctggccaggctcccaggctcgtcatctatggtgcatccaccagggtcactgctttcccagccaggttcactggcagtgggactgggacagagttcactctcaccatcagtagcctgcagtctgaagatcttgcagtttattactgtcagcagtattataggtggcctccgctcactttcggcggagggaccaaggtggaaatcaaac

SEQ ID NO: 92 - protein, 108 aa, Mus musculus
  MISC_FEATURE (1)..(108): amino acid sequence of IGL of anti-idiotypic Ab
EIVMTQSPATLSLSPGETATLSCRASHDINDNLAWYQQKPGQAPRLVIYGASTRVTAFPARFTGSGTGTEFTLTISSLQSEDLAVYYCQQYYRWPPLTFGGGTKVEIK

(19) United States
(12) Patent Application Publication
(10) Pub. No.: US 2022/0073963 A1
(43) Pub. Date: Mar. 10, 2022

(54) COMPOSITIONS AND METHODS FOR DETECTING AND TREATING TYPE 1 DIABETES AND OTHER AUTOIMMUNE DISEASES

(71) Applicant: THE JOHNS HOPKINS UNIVERSITY, Baltimore, MD (US)

(72) Inventors: Abdel Rahim Hamad, Ellicott City, MD (US); Thomas Donner, Baltimore, MD (US); Rizwan Ahmed, Baltimore, MD (US); Zahra Omidian, Baltimore, MD (US)

(21) Appl. No.: 17/416,778

(22) PCT Filed: Dec. 20, 2019
(86) PCT No.: PCT/US2019/067874

§ 371 Date: Jun. 21, 2021

Related U.S. Application Data
(60) Provisional application No. 62/854,286, filed on May 29, 2019.
(60) Provisional application No. 62/854,289, filed on May 29, 2019.
(60) Provisional application No. 62/782,624, filed on Dec. 20, 2018.
(60) Provisional application No. 62/782,646, filed on Dec. 20, 2018.

(51) Int. Cl. C12Q 1/686; C12Q 1/6881; C07K 16/28; C07K 16/42

(52) U.S. Cl. CPC C12Q 1/686; C12Q 1/6881; C07K 16/28; C12Q 2600/156; C07K 2317/76; C07K 2317/565; C07K 16/42

(57) ABSTRACT

The present invention relates to the field of diabetes. More specifically, the present invention provides compositions and methods useful for diagnosing and treating Type I diabetes. In one embodiment, a method comprises detecting a nucleotide sequence encoding SEQ ID NO:1 from a biological sample obtained from a patient. In another embodiment, the present invention provides an antibody or antigen-binding fragment thereof that specifically binds SEQ ID NO:1. In a further embodiment, the present invention provides an antibody or antigen-binding fragment thereof that specifically binds (i) a B-cell receptor expressed on a lymphocyte, wherein the B-cell receptor comprises SEQ ID NO:1; or (ii) a free-floating antibody comprising SEQ ID NO:1.

### CROSS-REFERENCE TO RELATED APPLICATIONS

This application claims the benefit of U.S. Provisional Application No. 62/854,289, filed May 29, 2019; U.S. Provisional Application No. 62/854,286, filed May 29, 2019; U.S. Provisional Application No. 62/782,646, filed Dec. 20, 2018; and U.S. Provisional Application No. 62/782,624, filed Dec. 20, 2018, each of which is incorporated herein by reference in its entirety.

### STATEMENT OF GOVERNMENTAL INTEREST

This invention was made with government support under grant no. AI099027, awarded by the National Institutes of Health. The government has certain rights in the invention.

### FIELD OF THE INVENTION

The present invention relates to the field of autoimmune disease. More specifically, the present invention provides compositions and methods useful for diagnosing and treating Type I diabetes.

### INCORPORATION-BY-REFERENCE OF MATERIAL SUBMITTED ELECTRONICALLY

This application contains a sequence listing. It has been submitted electronically via EFS-Web as an ASCII text file entitled “P14290-03_ST25.txt.” The sequence listing is 37,771 bytes in size, and was created on Dec. 20, 2019. It is hereby incorporated by reference in its entirety.

### BACKGROUND OF THE INVENTION

B and T cells are the two main lymphocytes of the adaptive immune system that work in concert to maintain host defense or cause autoimmunity in susceptible individuals. Expression of the B cell receptor (BCR) defines B cells and the T cell receptor (TCR) defines T cells. Both antigen receptors have similar structures and highly diverse repertoires (Wardemann et al., 2003). The BCR (surface immunoglobulin, Ig) is a heterodimer composed of heavy (IGH) and light (IGL) chains, whereas the αβ TCR heterodimer is composed of TCRα and TCR chains. Each receptor has a hypervariable region containing V (variable), D (diversity in case of IGH and TCRβ) and J (joining) gene segments randomly selected from large pools of unarranged segments and recombined to generate a complementarity determining regions (CDR3) that denotes the specificity of each clonotype and comprises its antigen binding site. The diversity is enhanced by N1 and N2 nucleotide additions/deletions at the V-D and the D-J junctions, respectively. Theoretically, up to 10″ unique BCRs or TCRs are generated during the development of B cells in bone marrow and T cells in thymus (Sewell, 2012). The diversity is essential for protecting host against virtually any pathogen, but it also leads to generation of autoreactive B and T cells that cause autoimmune diseases in genetically susceptible individuals. Currently, there is no cure for autoimmune diseases. A major reason is the limited knowledge about the identities of autoreactive lymphocytes and autoantigens that cause their activation. Clear understanding of autoreactive lymphocytes is expected to lead to antigen-specific immunotherapies that spare useful lymphocytes and host immune competence.

### SUMMARY OF THE INVENTION

In one aspect, the present invention provides compositions and methods for detecting the x-clonotype in patients. The x-clonotype is characterized by the following structure: VH04-b-N1-DH05-018-N2-J1-104-01*02 (including the N1 and N2 nucleotide addition) (see FIG. 3E). In one embodiment, a nucleotide sequence comprising or encoding the N-1-DH05-018-N2 region is detected. More specifically, in one embodiment, a method comprises detecting a nucleotide sequence encoding SEQ ID NO:1 from a biological sample obtained from a patient. In a specific embodiment, the detecting step comprises polymerase chain reaction (PCR). In certain embodiments, the PCR comprises amplification using primers comprising SEQ ID NOS:17-18. In other embodiments, the PCR comprises amplification using primers comprising SEQ ID NOS:23 and 19.

In certain embodiments, the nucleotide sequence comprises SEQ ID NO:25. In another embodiment, the nucleotide sequence comprises SEQ ID NO:2. Furthermore, a biological sample can include blood and other liquid samples of biological origin including, but not limited to, peripheral blood, serum, plasma, cerebrospinal fluid, urine, saliva, stool and synovial fluid. In a particular embodiment, the biological sample is peripheral blood.

In additional embodiments, the detecting step further comprises sequencing. In other embodiments, the methods further comprise the step of genotyping the HLA-DQ allele.

The present invention also provides methods for determining whether a patient is at-risk for Type 1 Diabetes (T1D) comprising the steps of (a) detecting a nucleotide sequence encoding SEQ ID NO:1 from a biological sample obtained from a patient; (b) genotyping the HLA-DQ to detect the presence of the HLA-DQ7 allele or the HLA-DQ8 allele, wherein a patient having SEQ ID NO:1 and HLA-DQ8 is at risk for T1D, a patient not having SEQ ID NO:1 and HLA-DQ7 is not at risk for T1D, and a patient not having SEQ ID NO:1 is not at risk for T1D. In certain embodiments, the compositions and methods of the present invention can be used to identify individuals at risk for developing T1D at different stages of disease development. In a specific embodiment, a cancer patient can be screened prior to checkpoint inhibitor treatment to identify the risk of developing T1D. Similar embodiments apply to screening for/risk of other autoimmune diseases. Indeed, the present invention can be used to assess autoimmune diseases including, but not limited to, rheumatoid arthritis, multiple sclerosis, systemic lupus erythematosus, Graves disease/Hashimoto's disease, inflammatory bowel diseases and rare autoimmune diseases like IgG4-related diseases and pemphigus vulgaris. In further embodiments, the methods of the present invention further comprises treating the patient. In particular embodiments, the treatment comprises administration of an antibody or antigen-binding fragments thereof as described herein.

In some embodiments, the treatment comprises administering insulin or pramlintide. Types of insulin include short-acting (regular) insulin, rapid-acting insulin, long-acting insulin and intermediate-acting insulin. Examples of short-acting (regular) insulin include Humulin R and Novolin R. Rapid-acting insulin examples include insulin glulisine (Apidra), insulin lispro (Humalog), insulin aspart (Novolog), Admelog, Agrezza inhaled powder, and Fiasp. Long-acting insulins include insulin glargine (Lantus, Toujeo Solostar), insulin detemir (Levemir), insulin degludec (Tresiba), and Basaglar. Intermediate-acting insulins include insulin NPH (Novolin N, Humulin N).

In another aspect, the present invention provides anti-idiotypic antibodies that bind to x-Id. In a specific embodiment, the present invention provides an isolated antibody or antibody-binding fragment thereof that specifically binds to x-Id, wherein the antibody or antibody-binding fragment comprises heavy chain complementarity determining regions (CDRs) 1, 2 and 3. In a further embodiment, the isolated antibody further comprises light chain CDRs 1, 2 and 3.

The present invention also provides an isolated antibody or antigen-binding fragment thereof that specifically binds to x-Id, wherein the antibody or antigen-binding fragment thereof comprises (a) a heavy chain variable region (VH) comprising CDR1, CDR2, and CDR; and (b) a light chain variable region (VL) comprising CDR1, CDR2, and CDR3.

In one embodiment, the present invention provides an antibody or antigen-binding fragment thereof that specifically binds SEQ ID NO:1. In another embodiment, the present invention provides an antibody or antigen-binding fragment thereof that specifically binds (i) a B-cell receptor expressed on a lymphocyte, wherein the B-cell receptor comprises SEQ ID NO:1; or (ii) a free-floating antibody comprising SEQ ID NO:1. In a further embodiment, an antibody or antigen-binding fragment thereof that specifically binds an antibody comprising SEQ ID NO:1. In certain embodiments, the antibody or antigen-binding fragment prevents or reduces the binding of antigen to SEQ ID NO:1. Furthermore, the antigen-binding fragment can comprise an scFv, sc(Fv)2, Fab, F(ab)2, and a diabody.

The present invention also provides an isolated nucleic acid molecule encoding the anti-x-Id antibody or antigen-binding fragment thereof. In a specific embodiment, a vector comprises a nucleic acid molecule described herein. In another embodiment, a host cell comprises a vector described herein. The host cell can be a prokaryotic or a eukaryotic cell. In particular embodiments, the present invention provides a method for producing an anti-x-Id antibody or antigen-binding fragment thereof comprising the steps of (a) culturing a host cell under conditions suitable for expression of the anti-x-Id antibody or antigen-binding fragment thereof by the host cells; and (b) recovering the anti-x-Id antibody or antigen-binding fragment thereof. The present invention also provides a composition comprising an anti-x-Id antibody or antigen-binding fragment thereof and a suitable pharmaceutical carrier. In particular embodiments, the composition is formulated for intravenous, intramuscular, oral, subcutaneous, intraperitoneal, intrathecal or intramuscular administration.

In another aspect, the present invention provides methods of treatment. In certain embodiments, the compositions and methods of the present invention can be used to target and inactivate T1D specific X-cells in persons having (a) high risk characteristics of developing T1D (i.e., those with high risk genetic profiles or T1D antibodies); (b) new onset T1D having residual endogenous insulin to preserve those remaining islet cells; (c) T1D to potentially enable pancreatic endodermal stem cells to regenerate islet cells; and (d) pancreatic or islet cell transplants, as part of an immunosuppressive medical regimen to block autoimmune attack on transplanted cells. The present invention can also be utilized to screen cancer patients prior to, for example, checkpoint inhibitor treatment to identify at risk for developing T1D or other autoimmune diseases.

In certain embodiments, a method for treating diabetes in a mammal comprises the step of administering to the mammal a therapeutically effective amount of the antibody or antigen-binding fragment thereof that specifically binds to x-Id. In one embodiment, a method for treating or preventing type 1 diabetes (T1D) in a subject having T1D or a risk thereof comprises the step of administering to the patient a therapeutically effective amount of an antibody or antigen-binding fragment described herein.

In further embodiments, the present invention can be used to treat other autoimmune diseases including, but not limited to, rheumatoid arthritis, multiple sclerosis, systemic lupus erythematosus, Graves disease/Hashimoto's disease, inflammatory bowel diseases and rare autoimmune diseases like IgG4-related diseases and pemphigus vulgaris.

In one embodiment, the present invention provides an isolated antibody or antibody-binding fragment thereof comprising heavy chain complementarity determining regions (CDRs) 1, 2 and 3, wherein the heavy chain CDR1 comprises an amino acid sequence as set forth in SEQ ID NO:60, or the amino acid sequence as set forth in SEQ ID NO:60 with a substitution at two or fewer amino acid positions, the heavy chain CDR2 comprising an amino acid set forth in SEQ ID NO:62, or the amino acid set forth in SEQ ID NO:62 with a substitution at two or fewer amino acid positions, and the heavy chain CDR3 comprising an amino acid sequence as set forth in SEQ ID NO:64, or the amino acid sequence as set forth in SEQ ID NO:64 with a substitution at two or fewer amino acid positions.

In a further embodiment, the isolated antibody or antigen-binding fragment further comprises light chain CDRs 1, 2 and 3, wherein the light chain CDR1 comprises an amino acid sequence as set forth in SEQ ID NO:66, or the amino acid sequence as set forth in SEQ ID NO:66 with a substitution at two or fewer amino acid positions, the light chain CDR2 comprising an amino acid sequence as set forth in SEQ ID NO:68, or the amino acid sequence as set forth in SEQ ID NO:68 with a substitution at two or fewer amino acid positions, and the light chain CDR3 comprising an amino acid sequence as set forth in SEQ ID NO:9, or the amino acid sequence as set forth in SEQ ID NO:9 with a substitution at two or fewer amino acid positions.

In another embodiment, the present invention provides an isolated antibody or antigen-binding fragment thereof that specifically binds a B-cell receptor expressed on a lymphocyte, wherein the B-cell receptor comprises SEQ ID NO:1, or a free-floating antibody comprising SEQ ID NO:1, wherein the antibody or antigen-binding fragment thereof comprises a heavy chain variable region (VH) comprising CDR1, CDR2, and CDR3 consisting of the amino acid sequences as set forth in SEQ ID NOS:60, 62 and 64, respectively.

In a further embodiment, an isolated antibody or antigen-binding fragment thereof that specifically binds a B-cell receptor expressed on a lymphocyte, wherein the B-cell receptor comprises SEQ ID NO:1, or a free-floating antibody comprising SEQ ID NO:1, wherein the antibody or antigen-binding fragment thereof comprises a light chain variable region (VL) comprising CDR1, CDR2, and CDR3 consisting of the amino acid sequences as set forth in SEQ ID NOS: 66, 68 and 9, respectively.

The present invention also provides an isolated antibody or antigen-binding fragment thereof that specifically binds (1) a B-cell receptor expressed on a lymphocyte, wherein the B-cell receptor comprises SEQ ID NO:1, or (2) a free-floating antibody comprising SEQ ID NO:1, wherein the antibody or antigen-binding fragment thereof comprises (a) a VH comprising CDR1, CDR2, and CDR3, consisting of the amino acid sequences as set forth in SEQ ID NOS:60, 62 and 64, respectively; and (b) a VL comprising CDR1, CDR2, and CDR3, consisting of the amino acid sequences as set forth in SEQ ID NOS:66, 68 and 9.

In a further embodiment, an isolated antibody or antigen-binding fragment thereof that specifically binds (1) a B-cell receptor expressed on a lymphocyte, wherein the B-cell receptor comprises SEQ ID NO:1, or (2) a free-floating antibody comprising SEQ ID NO:1, wherein the antibody or antigen-binding fragment thereof comprises a VH comprising CDRs 1, 2, and 3 with the amino acid sequences set forth in SEQ ID NOS:60, 62 and 64, respectively and a VL comprising CDRs 1, 2, and 3 with the amino acid sequences set forth in SEQ ID NOS:66, 68 and 9, respectively. In certain embodiments, the antigen-binding fragment is selected from the group consisting of an scFv, sc(Fv)2, Fab, F(ab)2, and a diabody.

The present invention also provides an isolated antibody or antigen-binding fragment thereof that specifically binds (1) a B-cell receptor expressed on a lymphocyte, wherein the B-cell receptor comprises SEQ ID NO:1, or (2) a free-floating antibody comprising SEQ ID NO:1, wherein the antibody or antigen-binding fragment thereof comprises a VH comprising the amino acid sequence set forth in SEQ ID NO:74.

In another embodiment, an isolated antibody or antigen-binding fragment thereof that that specifically binds (1) a B-cell receptor expressed on a lymphocyte, wherein the B-cell receptor comprises SEQ ID NO:1, or (2) a free-floating antibody comprising SEQ ID NO:1, wherein the antibody or antigen-binding fragment thereof comprises a VL comprising the amino acid sequence as set forth in SEQ ID NO:76.

In particular embodiments, an isolated antibody or antigen-binding fragment thereof comprises a VH comprising the amino acid sequence as set forth in SEQ ID NO:74 and a VL comprising the amino acid sequence as set forth in SEQ ID NO:76. In certain embodiments, the antibody or antigen-binding fragment thereof is humanized.

In one embodiment, the present invention provides an isolated antibody or antibody-binding fragment thereof comprising heavy chain complementarity determining regions (CDRs) 1, 2 and 3, wherein the heavy chain CDR1 comprises an amino acid sequence as set forth in SEQ ID NO:78, or the amino acid sequence as set forth in SEQ ID NO:78 with a substitution at two or fewer amino acid positions, the heavy chain CDR2 comprising an amino acid set forth in SEQ ID NO:80, or the amino acid set forth in SEQ ID NO:80 with a substitution at two or fewer amino acid positions, and the heavy chain CDR3 comprising an amino acid sequence as set forth in SEQ ID NO:82, or the amino acid sequence as set forth in SEQ ID NO:82 with a substitution at two or fewer amino acid positions.

In a further embodiment, the isolated antibody or antigen-binding fragment further comprises light chain CDRs 1, 2 and 3, wherein the light chain CDR1 comprises an amino acid sequence as set forth in SEQ ID NO:84, or the amino acid sequence as set forth in SEQ ID NO:84 with a substitution at two or fewer amino acid positions, the light chain CDR2 comprising an amino acid sequence as set forth in SEQ ID NO:86, or the amino acid sequence as set forth in SEQ ID NO:86 with a substitution at two or fewer amino acid positions, and the light chain CDR3 comprising an amino acid sequence as set forth in SEQ ID NO:88, or the amino acid sequence as set forth in SEQ ID NO:88 with a substitution at two or fewer amino acid positions.

In another embodiment, the present invention provides an isolated antibody or antigen-binding fragment thereof that specifically binds a B-cell receptor expressed on a lymphocyte, wherein the B-cell receptor comprises SEQ ID NO:1, or a free-floating antibody comprising SEQ ID NO:1, wherein the antibody or antigen-binding fragment thereof comprises a heavy chain variable region (VH) comprising CDR1, CDR2, and CDR3 consisting of the amino acid sequences as set forth in SEQ ID NOS:78, 80 and 82, respectively.

In a further embodiment, an isolated antibody or antigen-binding fragment thereof that specifically binds a B-cell receptor expressed on a lymphocyte, wherein the B-cell receptor comprises SEQ ID NO:1, or a free-floating antibody comprising SEQ ID NO:1, wherein the antibody or antigen-binding fragment thereof comprises a light chain variable region (VL) comprising CDR1, CDR2, and CDR3 consisting of the amino acid sequences as set forth in SEQ ID NOS: 84, 86 and 88, respectively.

The present invention also provides an isolated antibody or antigen-binding fragment thereof that specifically binds (1) a B-cell receptor expressed on a lymphocyte, wherein the B-cell receptor comprises SEQ ID NO:1, or (2) a free-floating antibody comprising SEQ ID NO:1, wherein the antibody or antigen-binding fragment thereof comprises (a) a VH comprising CDR1, CDR2, and CDR3, consisting of the amino acid sequences as set forth in SEQ ID NOS:78, 80 and 82, respectively; and (b) a VL comprising CDR1, CDR2, and CDR3, consisting of the amino acid sequences as set forth in 84, 86 and 88.

In a further embodiment, an isolated antibody or antigen-binding fragment thereof that specifically binds (1) a B-cell receptor expressed on a lymphocyte, wherein the B-cell receptor comprises SEQ ID NO:1, or (2) a free-floating antibody comprising SEQ ID NO:1, wherein the antibody or antigen-binding fragment thereof comprises a VH comprising CDRs 1, 2, and 3 with the amino acid sequences set forth in SEQ ID NOS:78, 80 and 82, respectively and a VL comprising CDRs 1, 2, and 3 with the amino acid sequences set forth in SEQ ID NOS:84, 86 and 88, respectively. In certain embodiments, the antigen-binding fragment is selected from the group consisting of an scFv, sc(Fv)2, Fab, F(ab)2, and a diabody.

The present invention also provides an isolated antibody or antigen-binding fragment thereof that specifically binds (1) a B-cell receptor expressed on a lymphocyte, wherein the B-cell receptor comprises SEQ ID NO:1, or (2) a free-floating antibody comprising SEQ ID NO:1, wherein the antibody or antigen-binding fragment thereof comprises a VH comprising the amino acid sequence set forth in SEQ ID NO:90.

In another embodiment, an isolated antibody or antigen-binding fragment thereof that that specifically binds (1) a B-cell receptor expressed on a lymphocyte, wherein the B-cell receptor comprises SEQ ID NO:1, or (2) a free-floating antibody comprising SEQ ID NO:1, wherein the antibody or antigen-binding fragment thereof comprises a VL comprising the amino acid sequence as set forth in SEQ ID NO:92.

In particular embodiments, an isolated antibody or antigen-binding fragment thereof comprises a VH comprising the amino acid sequence as set forth in SEQ ID NO:90 and a VL comprising the amino acid sequence as set forth in SEQ ID NO:92.

In a further embodiment, an isolated antibody or antigen-binding fragment thereof comprises a VH comprising CDRs 1, 2, and 3 with the amino acid sequences set forth in SEQ ID NOS:60, 62 and 64, respectively and a VL comprising CDRs 1, 2, and 3 with the amino acid sequences set forth in SEQ ID NOS:84, 86 and 88, respectively. In particular embodiments, an isolated antibody or antigen-binding fragment thereof comprises a VH comprising the amino acid sequence as set forth in SEQ ID NO:74 and a VL comprising the amino acid sequence as set forth in SEQ ID NO:92.

In a further embodiment, an isolated antibody or antigen-binding fragment thereof comprises a VH comprising CDRs 1, 2, and 3 with the amino acid sequences set forth in SEQ ID NOS:78, 80 and 82, respectively and a VL comprising CDRs 1, 2, and 3 with the amino acid sequences set forth in SEQ ID NOS:66, 68 and 9, respectively. In particular embodiments, an isolated antibody or antigen-binding fragment thereof comprises a VH comprising the amino acid sequence as set forth in SEQ ID NO:90 and a VL comprising the amino acid sequence as set forth in SEQ ID NO:76.

In certain embodiments, the described antibodies or antigen-binding fragments thereof are humanized.

In a further aspect, the present invention provides a vaccine. In particular embodiments, the x-peptide or derivative thereof (SEQ ID NO:1) can be used as an immunogen to neutralize, inactivate, destroy or cause anergy of insulin-reactive T cells.

### BRIEF DESCRIPTION OF THE FIGURES

FIG. 1A-1F. A rare subset of lymphocytes coexpresses TCR and BCR and expands in T1D. (FIG. 1A) Representative dot plots show coexpression of IgD and TCR among gated CD5⁺ CD19⁺ cells in T1D (Top panel) and HC (bottom panel) subjects. Numbers indicate percentages in quadrants. Graph shows cumulative data (Mean±SEM). Each dot represents one donor, T1D (red, n=16); HC (black, n=11); ***P<0.001, ****p<0.0001 by Two-way ANOVA with Sidak's multiple comparisons test (see also FIG. 8). (FIG. 1B) Representative AMNIS images show coexpression of IgD, TCR and IgM by gated single IgD⁺ DEs versus their differential expression in B_{con} and T_{con} cells in three T1D subjects (n=32 DE cells). BF, bright field (see also FIG. 9). (FIG. 1C) Heatmap of genes differentially expressed by DEs, B_{con} or T_{con} cells. Top row shows cell types. Subsequent three rows show expression of ACTB, PPIA, and UBB housekeeping genes followed by the top 30 genes preferentially expressed in each cell type. The color scale indicates the gene expression in log 2(RSEM+1). Note that DEs differentially express large numbers of genes that are absent or low in B_{con} and T_{con} cells. DEs share expression of markers of B and T cells with respective cell type. Data from 33 DEs (green), 20 B_{con} cells and 24 T_{con} cells. (FIG. 1D) Heatmap shows DEs shared expression of indicated lineage markers with respective cell type T_{con} or B_{con} cells. (FIG. 1E) Heatmap shows DEs shared expression of Igα (CD79a) and Igβ (CD79b) with B_{con} cells and CD3 subunits with T_{con} cells. CD247 is CD3zeta. (FIG. 1 F) Reconstruction of BCR and TCR in four DEs. No dual expression of BCR and TCR noted among T_{con} and B_{con} cells.

FIG. 2A-2D. TCR-activated DEs maintain their dual phenotype and upregulate MHC and costimulatory molecules (see also FIGS. 9, 10, and 11). (FIG. 2A) TCR activation leads to the upregulation of CD69 by IgD⁺ and IgD⁻ cells. Left dot plots show gating of CD5+CD19+ cells and B_{con} and T_{con} cells in anti-CD3/CD28 (top panel) and unstimulated control (bottom panel) cultures. Middle dot plots show expression of TCR and IgD by gated subsets. Overlays and graph show CD69 expression by gated IgD⁺ (red) and IgD⁻ (navy blue); and T_{con} cells (green), B_{con} cells (blue) in activated and control cultures. Each dot represents one donor, (n=5); ****p<0.0001 by Two-way ANOVA with Tukey's multiple comparisons test. (FIG. 2B) TCR activation leads to the proliferation of IgD⁺ and IgD⁻ DE subsets and T_{con} cells as determined by CFSE dilution. Open histograms denote unstimulated cultures. (FIG. 2C) Upregulation of HLA-DR and DQ by TCR-activated DEs. Note that B_{con} cells were present in control but not activated cultures. Graphs show MFI (Mean±SEM) for HLA-DR (left) and HLA-DQ (right). Each dot represents one donor, (n=4); **p<0.01 by Two-way ANOVA with Sidak's multiple comparisons test. See FIG. 10 for upregulation of costimulatory molecules. (FIG. 2D) DEs maintain Ig isotype phenotypes after 7 days of anti-CD3/CD28 stimulation.

FIG. 3A-3J. IGHV repertoires of DEs are predominated by one clonotype in T1D subjects (see also FIG. 12 and Tables 1, 4, 5, 6, and 7 (Tables not shown)). (FIG. 3A-3C) Venn diagrams show VH gene usage by IgD⁺ (red) and IgD⁻ (yellow) DEs and B_{con} cells (blue) in T1D #1, #2 and #3 patients. Graphs show percentages of the top 10 VH (or all 7 VH genes in the case of T1D #2) genes used by IgD⁺ or IgD⁻ DEs as compared to B_{con} cells in each patient. Arrows point to the IGHV-04-b⁺ gene segment which was predominantly used by IgD⁺ and IgD⁻ DEs in the three patients. (FIG. 3D) Graph shows absolute number of mutations per VH gene in DEs and B_{con} cells in the three T1D subjects. Each dot represents an individual VH gene. (FIG. 3E) Schematic shows the V_{H}(N1)D(N2)J_{H} structure with the nucleotide and amino acid sequences of the CDR3 of the x-clonotype. (FIG. 3F) Venn diagram shows that the x-clonotype is one of two (red) clonotypes shared among B_{con} cells of the three T1D subjects. (FIG. 3G) Venn diagram shows diverse VH gene usage by IgD⁺ (red) and IgD⁻ (yellow) DEs comparable to that of B_{con} (blue) in HC #1. Graph shows percentages of the top 10 VH genes used by IgD⁺ DEs as compared to IgD⁻ DEs and B_{con} cells. (FIG. 3H) Comparison of CDR3 sequences of IGHV04-b⁺ clonotypes in the three T1D subjects and HC #1. *Indicates gap in sequence. Note that the highly conserved usage of VH04-b and JH04-01*02 by DEs in all subjects. (FIG. 3I) Number of mutations per VH gene in DE cells and B_{con} cells. Each dot represents one VH gene. (FIG. 3J) Schematic shows primers used for detection of x-clonotype in peripheral blood of genotyped T1D and HCs. Table shows detection of x-clonotype in PBMC cDNAs of T1D and HC subjects using sequence-specific primers. Note x-clonotype is detectable in DQ7⁺ (β57D⁺ isoform of DQ8), but not DQ8⁺ and DQ2⁺ HCs. A second probe with astringent reverse primer design produced similar results (Table 7, not shown).

FIG. 4A-4J. HLA-CDR3 peptide binding (see also FIG. 13). (FIG. 4A-B) HLA molecule loaded with (FIG. 4A) CDR3 (x-Id) peptide (CARQEDTAMVYYFDYW) (SEQ ID NO:1) and (FIG. 4B) Superagonist (SHLVEELYLVAGEEG) (SEQ ID NO:7) from Wang et al. 2018. HLA-α is shown in cyan cartoon, HLA-β is shown in silver cartoon, epitope residues are colored by type: white hydrophobic, green polar, blue basic, red acidic. (FIG. 4C) Change in binding affinity for mutating from polyglycine to the epitope for the CDR3 peptide and superagonist. (FIG. 4D) Binding affinity decomposition into vdW and electrostatics (Coulomb) for the CDR3 (x-Id) Peptide and Superagonist. (FIG. 4E) Van der Waals interaction energy between the HLA and epitope from Molecular Dynamics (MD) simulation. (FIG. 4F-G) Percentage of epitope residues buried in HLA for (FIG. 4F) CDR3 (x-Id) peptide (CARQEDTAMVYYFDYW) (SEQ ID NO:1) and (FIG. 4G) Superagonist (SHLVEELYLVAGEEG) (SEQ ID NO:7) from Wang et al. 2018. The sequence in bold is the ‘core epitope’ sequence discussed in the text. (FIG. 4H) Average fluctuation (RMSF) for each residue in A. (FIG. 4I) Detailed structure of buried salt bridges between CDR3 peptide and HLA. Basic residues in blue, acidic in red, epitope backbone in tan. (FIG. 4J) Left, overlay of most representative epitope conformations for the CDR3 peptide (light blue) and superagonist (red) with tyrosine residues in pocket 6 and 7 for the CDR3 peptide highlighted. Right, side view, showing similar P1, P4, and P9 agreement, but large differences elsewhere. Note: (FIG. 4C-4D) Error bars are standard error across 6 replicas. (FIG. 4E-4H) Error bars are standard error from dividing the last 250 ns of MD simulation into 5 sections.

FIG. 5A-5C. x-Id peptide forms functional HLA-DQ8 complexes that stimulate CD4 T cells (see also FIG. 14). (FIG. 5A) Representative silver-stained SDS gel shows binding of indicated peptides to soluble DQ8 to form stable heterodimers. Arrows indicate p/DQαβ dimers and DQα and DQβ monomers, respectively. The results are from one of three independent experiments with similar results. (FIG. 5B) x-Id/DQ8 complexes stimulate proliferation of CD4 T cells from DQ8⁺ T1D. Representative dot plots show CFSE dilution by gated CD4 T cells among PBMCs from in T1D or HC subjects that were stimulated with indicated peptide-DQ8 complexes. Numbers indicate percentages of gated CFSE^{low} CD4 T cells. Dot plots on the right show inhibition of proliferation by anti-DQ mAb. Graph shows cumulative data from three DQ8⁺ T1D and three HC subjects, (n=3); *p<0.05 by Two-way ANOVA with Sidak's multiple comparisons test. (FIG. 5C) Overlays show upregulation of CD69 by gated CFSE^{low} CD4 T cells (red line) versus CFSE^{hi} CD4 T cells (green line) in each subject group. Numbers indicate percentages (Mean±SEM) of CFSE^{low} CD4 T cells.

FIG. 6A-6B. Verification of dual expression of BCR and TCR by DEs using an EBV-immortalized clone. (FIG. 6A) Schematic depicts generation of lymphoblastoid cell line (x-LCL) and analysis of its cells for encoded antibody using two approaches: 1. Cloning from sorted single cells (n=7 cells) that yielded two antibodies that shared expression of the x-clonotype paired with one of two (IGL1, IGL2) light chains. 2. Usage of limiting dilution to generate the x1.1 clone and use of PCR cloning to amplify transcripts of BCR (IgL1/x-clonotype) and TCRαβ followed by usage of IMGTV-Quest to identify VDJ usage and CDR3 sequences. Nucleotide sequences of cloned receptors are shown. (FIG. 6B) Naturally produced x-mAb^{N} stimulates CD4 T cells from T1D. Coommassie blue-stained gel shows production of xmAb^{N} by the x1.1 clone. Arrows point to heavy and light chains of x-mAbN (of IgM isotype). Representative plots show dilution of CFSE by CD4 T cells activated by soluble x-mAb^{N}. Numbers indicate percentages (Mean±SEM) of CFSE^{low} CD4 T cells (n=3).

FIG. 7A-7C. Recombinant x-mAbR cross-activates insulin-reactive CD4 T cells. (FIG. 7A) Schematic depicts amplification, cloning and CDR3 sequences of the light and heavy chain of x-mAb^{R} from a single DE cell and expression using IgG-AbVec and Igλ-AbVec expression vectors. (FIG. 7B) Silver-stained reduced gel shows heavy and light chains (arrows) of the x-mAb^{R}. Representative plots show dilution of CFSE by activated CD4 T cells stimulated with immobilized x-mAb^{R}. Numbers indicate percentages (Mean±SEM) of CFSE^{low} CD4 T cells (n=5). (FIG. 7C) Binding inhibition indicates overlapping of x-Id and mimotope-reactive CD4 T cells. x-mAb^{R} inhibits binding of mim-tet and x-Id-tet to CD4 T cells that had been activated with x-Id or mimotope. PBMCs were cultures for 7 days in the presence of absence of x-Id or mimotope peptide. Top dot plots show that CD4 T cells expanded by the x-Id-peptide are detectable not only by x-Id-tet, but also by mim-tet. Reciprocally, CD4 T cells expanded by the mimotope peptide are detectable by both the x-Id-tet and mim-tet. CLIP-Tet was used to measure background staining and x-Id-tet⁺ or mim-tet⁺ in unstimulated cultures identify precursor frequencies. Bottom dot plots show that preincubating with cells with x-mAb^{R} inhibits tetramer staining. Left graph shows frequency of tetramer+ CD4 T cells in different cultures of x-Id peptide-stimulated cultures. Right graph shows data from mimoptope-stimulated cultures. Each line represents one donor. Blockade with x-mAb^{R} inhibited tetramer binding, (n=3); *P<0.01, ***P<0.001, ****p<0.0001 by Two-way ANOVA with Tukey's multiple comparisons test.

FIG. 8A-8H. Verification of DEs using different specificity controls, Related to FIG. 1. Single cell suspensions were surface-stained for 20 min on ice with predetermined concentrations of indicated fluorochrome-conjugated antibodies. Acquired samples (5×105 to 1×106 live events) were properly compensated using single color stains. Data analysis, gating, and graphical presentation were done using FlowJo software (TreeStar). (FIG. 8A) Live lymphocytes were gated and doublets excluded using FSC-Height versus FSC-Width and SSC-Height versus SSC-Width plots. Our analysis also included using the following controls: (FIG. 8B) No DEs were detected in unstained samples, providing specificity control for autofluroescence. (FIG. 8C) Fluorescence-Minus One (FMO) analyses show no nonspecific signals for CD5, TCR, IgD, and CD19 respectively. (FIG. 8D) No nonspecific IgD or TCR signals were detected using PE-IgG1 and AF-488-IgG2a isotype controls, respectively. (FIG. 8E) Inclusion of FC-blockade during staining did not alter detection of DEs. (FIG. 8F) Exclusion of CD11b⁺ monocytes using dump channel did not alter detection of DEs. (FIG. 8G) Dot plots show detection of DEs using the above controls. (FIG. 8H) To exclude that DEs were a consequence of non-specific conjugate formation between B and T cells, we sorted and cultured alone or together in the presence or absence of anti-CD3/CD28 stimulation. NO dual expressers were detected. Similar results were obtained from the two experiments and four replicate cultures. Also see FIGS. 12A and 12B.

FIG. 9A-9F. DEs coexpress pan-markers of T and B cells and functional BCR, Related to FIGS. 1 and 2. (FIG. 9A) Representative AMNIS images show coexpression of CD40 and CD40L by DE and their differential expression by B_{con} and T_{con} cells, respectively. Similar results were obtained from individual 47 DE cells. (FIG. 9B) Representative AMNIS images show expression of CD28 by DE and T_{con} cell, but not B_{con} cell. Similar results were obtained from individual 11 DE cells. (FIG. 9C) Representative dot plots show that gated IgD⁺ and IgD⁻ DEs are comprised of CD4⁺, CD8⁺ and CD4⁻ CD8⁻ double negative (DN) subsets using T_{con} cells as a control. Graph shows cumulative data (Means±SEM), n=5. (FIG. 9D) Representative AMNIS images show expression of CD4, CD8 or lack of both by single DE cells. Similar results were obtained from individual 42 DE cells. (FIG. 9E) Overlay plot shows expression of CD3 by gated by IgD⁺ and IgD⁻ DE cells as compared to T_{con} cells, using B_{con} cells as negative control. Graph shows cumulative data (Mean±SEM) from three donors, ns, not statistically significant. (FIG. 9F) Representative overlays show CD79a phosphorylation in different cell types at indicated time points after anti-IgM stimulation. Numbers indicate MFI, B_{con} (blue), IgD⁺ DEs (red), IgD⁻ (navy blue) and T_{con} cells (green). Graph shows cumulative data (Mean±SEM, n=3). *p<0.05, ***p<0.001 by Two-way ANOVA with Sidak's multiple comparison test. Significance relative to time zero.

FIG. 10A-10B. DEs coexpress pan-markers of B and T cells and particularly upregulate B cell markers in response to anti-CD3/CD28 stimulation, Related to FIG. 2. PBMCs from T1D subjects were analyzed for the expression of indicated molecules immediately ex vivo or after 7 days of stimulation with immobilized anti-CD3/CD28 mAbs. (FIG. 10A) Histograms show representative expression of indicated molecules ex vivo and after 7 days of stimulation. Graphs show MFI of indicated molecules. Each symbol represents one subject (n=4) from four independent experiments. **p<0.01, ***p<0.001 by Two-way ANOVA with Sidak's multiple comparison test. Note, B_{con} cells were too few in stimulated cultures, hence not examined in activated cultures. (FIG. 10B) Dot plots show that TCR-activated subsets of DEs maintain expression CD45RA do not switch to CD45RO. Note that majority of gated T_{con} cells expressed CD45RO. Representative results are from one of three independent experiments with similar results.

FIG. 11A-11D. Rapid cytokine production subsets of DE cells in response to PMA/ionomycin or TCR stimulation, Related to FIG. 2. (FIG. 11A) PMA/ionomycin stimulation induce significant intracellular production of IL-10 and IFN-γ by DE cells. Top panel, left dot plot shows gating of B_{con} or T_{con} cells and CD5⁺CD19⁺ cells after 4 h stimulation of PBMCs with PMA/ion. Right dot plot show division of gated CD5⁺CD19⁺ cells into DE (TCR⁺) and TCR⁻ subpopulations. Middle panel shows expression of intracellular IL-10 by each subset. Bottom panel shows expression of intracellular IFN-γ by each subpopulation. Numbers indicate percentages in indicated quadrants. Graphs show cumulative data (Mean±SEM). Each dot represents one subject, n=3; ***p<0.001, ****p<0.0001 by one-way ANOVA with Tukey's multiple comparison test. Cells in unstimulated cultures did not produce cytokines and were used to draw quadrants delineating the boundaries between positive and negative cells. (FIG. 11B-11D) TCR activation induces expression of IL-10 and IFN-γ by DEs without a need for secondary PMA/ion stimulation. PBMCs from T1D donors (n=3) were cultured in the presence (stimulated) or absence (control) of immobilized anti-CD3/CD28 mAbs for 7 days and analyzed for intracellular cytokine analysis without or after 4 h stimulation with PMA/ion. (FIG. 11B) Representative dot plots show intracellular expression of IL-10 and IFN-γ by anti-CDR3/CD28-activated DE cells in the absence of secondary stimulation with PMA/ion. Graphs show cumulative data (Mean±SEM, n=3). Each dot represents one subject, n=3; *p<0.05 by one-way ANOVA with Tukey's multiple comparison test. (FIG. 11C) Representative dot plots show intracellular expression of IL-10 and IFN-γ by anti-CDR3/CD28-activated DE cells after secondary stimulation with PMA/ion. Note, PMA/ion stimulation enhances IFN-γ production by TCR-stimulated T_{con} cells, which still failed to produce significant IL-10. Graphs show cumulative data (Mean±SEM). Each dot represents one subject, n=3; *p<0.05 by one-way ANOVA with Tukey's multiple comparison test. (FIG. 11D) Representative dot plot show intracellular cytokines production by gated cell types in unstimulated cultures. Graphs show cumulative data (Mean±SEM).

FIG. 12A-12J. Sorting strategy, high-throughput analysis of TCRBV repertoires of DEs and top 10 IGHV used by B_{con} cells, Related to FIG. 3. (FIG. 12A) Strategy used to isolate DEs for high-throughput analysis. Dot plots show gating of lymphocytes, exclusion of doublets and dead cells followed by using of CD5 and CD19 expression to divide live singlets into B_{con}, T_{con} cells and DE cells which were identified based on TCR expression among CD5⁺CD19⁺ cells. DE cells were further divided into IgD⁺ and IgD⁻ subsets and sorted accordingly. (FIG. 12B) Dot plots show purity check of sorted DEs, B_{con} and T_{con} cells. Purity of IgD⁻ cells was not tested due to cell limitation. (FIG. 12C-12F) Restricted TCRVβ usage by DE cells. (FIG. 12C-12E) Venn diagrams show TCRBVβ usage by IgD⁺ (red) and IgD− (yellow) subsets of DE cells versus that of T_{con} cells (green) from T1D #1, #4, and #5, respectively. Graphs show percentages of the top 10 TCRBV genes used by IgD⁺ DE cells as compared to their percentages in IgD⁻ DE cells and T_{con} cells in each of the three subjects. (FIG. 12F) Venn diagram and graph show TCRBV usage and all 7 TCRBV genes used in IgD⁺ cells in HC #1 and their percentages in IgD⁻ and B_{con} cells in HC #1. (FIG. 12G-12J) Top 10 VH genes used by B_{con} cells in T1D subjects do not include the IGHV04-b⁺ clonotypes. Graphs shows frequency distributions of the top 10 VH genes in B_{con} as compared to their percentages, when applicable, to IgD⁺ (red) and IgD⁻ (yellow) subsets of DE cells versus that of T_{con} cells (blue) in the three T1D (FIG. 12G-12I) and HC #1 (FIG. 12J).

FIG. 13A-13H. HLA-epitope RMSD and structure, Related to FIG. 4. (FIG. 13A) HLA-healthy control (CARQERFWSGPLFDYW) (SEQ ID NO:1) epitope structure. HLA-α is shown in cyan cartoon, HLA-β is shown in silver cartoon, epitope residues are colored by type: white hydrophobic, green polar, blue basic, red acidic. (FIG. 13B) HLA-epitope root mean square deviation of atomic positions (RMSD) of the HLA-epitope complexes over simulation time. (FIG. 13C-13D) HLA RMSD values of the (FIG. 13C) HLA-α and (FIG. 13D) HLA-β chains showing instability in the HLA-β chain for the healthy control epitope. (FIG. 13E) Epitope RMSD. RMSD plots are 1 ns running averages of backbone atoms. (FIG. 13F-13H) Prominent HLA-epitope interactions. (FIG. 13F) Tyrosine site 6 (orange) of the CDR3 peptide making numerous π-π interactions with Phe11, Tyr30, and Trp61 of HLA-β in this strongly aromatic pocket. (FIG. 13G) Tyrosine site 7 (orange) of the CDR3 peptide has the highest % contact area for the CDR3 peptide. Here, the hydrophobic, aromatic ring makes large contacts with Val67 and Tyr47 while the hydroxyl group contacts Thr71 and Arg70, all on HLA-β. (FIG. 13H) Tyrosine site 3 (orange) of the superagonist has the highest % contact area for the superagonist. Here, tyrosine makes extensive contacts with other aromatic residues including Phe54, His24, Tyr22, Tyr9, and Phe58 on HLA-α. HLA-α is shown in cyan cartoon, HLA-β is shown in silver cartoon, backbone of the CDR3 peptide is shown in magenta, residues are colored by type: white hydrophobic, green polar, blue basic.

FIG. 14A-14B. Soluble idiotope peptide (x-Id) stimulates CD4 T cells from T1D, but not HC, subjects, Related to FIG. 5. Freshly isolated CFSE-labelled PBMCs from T1D and HC subjects were cultured in the presence or absence of indicated peptides for 7 days. Samples were stained, acquired and analyzed by FlowJo. CD4 T cells were gated and percentage of divided (CFSElow T cells) were determined. (FIG. 14A) Dot plots show representative dilution of CFSE by activated CD4 T cells in the two groups, numbers indicate percentages of CFSElow CD4 T cells. Anti-DQ8 mAb inhibited proliferation in response to x-Id or mimotope (the two strongest simulant). Anti-DR blockade did not inhibit proliferation (data not shown). Graph shows cumulative data (Mean±SEM). All peptides induced significant proliferation from diabetogenic subjects compared to HCs. n=4 per subject group, *p<0.05 by Two-way ANOVA with Sidak's multiple comparisons test. Note that h-Id peptide from DEs of HC #1 did not elicit proliferation of CD4 T cells from either T1D or HCs. (FIG. 14B) Overlays show upregulation of CD69 by gated CFSElow T cells (red lines) versus CFSEhi non-proliferating (green) CD4 T cells from three subjects and three independent experiments. Numbers indicate percentages (Mean±SEM) of CFSElow CD4 T cells.

### DETAILED DESCRIPTION OF THE INVENTION

It is understood that the present invention is not limited to the particular methods and components, etc., described herein, as these may vary. It is also to be understood that the terminology used herein is used for the purpose of describing particular embodiments only, and is not intended to limit the scope of the present invention. It must be noted that as used herein and in the appended claims, the singular forms “a,” “an,” and “the” include the plural reference unless the context clearly dictates otherwise. Thus, for example, a reference to a “protein” is a reference to one or more proteins, and includes equivalents thereof known to those skilled in the art and so forth.

Unless defined otherwise, all technical and scientific terms used herein have the same meaning as commonly understood by one of ordinary skill in the art to which this invention belongs. Specific methods, devices, and materials are described, although any methods and materials similar or equivalent to those described herein can be used in the practice or testing of the present invention.

All publications cited herein are hereby incorporated by reference including all journal articles, books, manuals, published patent applications, and issued patents. In addition, the meaning of certain terms and phrases employed in the specification, examples, and appended claims are provided. The definitions are not meant to be limiting in nature and serve to provide a clearer understanding of certain aspects of the present invention. The present invention is based, at least in part, on the identification of an unexpected population of autoreactive lymphocytes that violates the paradigm that the split of adaptive lymphocytes into T and B cells is absolute. These lymphocytes are referred to as dual expressers (DEs) due to their coexpression of TCR and BCR and lineage markers of both B and T cells.

More specifically, analysis of DEs in peripheral blood of type 1 diabetes (T1D) subjects revealed a previously unknown neoantigen that is encoded in the heavy chain of a BCR that is clonally expanded in these patients. Discovery of this neoantigen could explain how insulin-reactive CD4 T cells are primed—one of the most important but poorly understood aspects of T1D. On the one hand, development of T1D is tightly linked to a polymorphism at (357 position of the β chain of the HLA-DQ (DQ8 and DQ2) major histocompatibility class II molecules that replaces Aspartic acid (D) by Alanine (β57D⁻) in ˜90% of T1D patients (Nakayama et al., 2015). On the other hand, naturally-processed epitopes of the insulin B:9-23 peptides (SHLVEALYLVCGERG) (SEQ ID NO:16), the primary T1D autoantigen, have low binding ability to these alleles and are potent autoantigens. Efforts to explain this paradox has centered on the fact that these DQ alleles favor antigenic peptides with negatively charged resides at positions 9 (P9) and P1 (Chang and Unanue, 2009). But naturally processed insulin B:9-23 peptide places non-acidic residues (V or A) at P1 and (G or R) at P9, providing a rationale for it is poor binding to DQ molecules. Hence, posttranslational modification has been suggested as a mechanism that generates potent insulin epitope. In support of this hypothesis, substitution of arginine (R) at P9 by glutamic acid (R22E) generates a potent mimotope that is 100-fold more potent than native insulin B:9-23 (Wang et al., 2018). Moreover, combining R22E with A14E substitution at the P1 position generates a superagonist that is about 1000-fold more potent than native insulin B:9-23 (Wang et al., 2018). Insulin peptides with such modifications, however, have not been identified in vivo. As described herein, we identified a potent neoantigen (x-Id peptide) with optimal anchor residues for DQ8 that is encoded in the idiotype of DEs that were clonally expanded in T1D subjects. In concordance, synthesized x-Id peptide forms stable DQ8 complexes and potently stimulates autoreactive CD4 T cells from T1D, but not healthy controls. Moreover, x-clonotype-bearing mAbs stimulate CD4 T cells and inhibited insulin-tetramer binding to CD4 T cells. Taken together, the present invention demonstrates that compartmentalization of T and B cells is not absolute and violators of this paradigm could be involved in driving autoimmunity.

Accordingly, in one aspect, the present invention describes an isolated antibody that can be used to detect and activate autoreactive T cells in T1D. In certain embodiments, the antibody or antigen-binding fragment thereof comprises a heavy chain comprising SEQ ID NO:28. In a further embodiment, the antibody or antigen-binding fragment further comprises a light chain comprising SEQ ID NO:30. The antibody or antigen-binding fragment can further comprise a light chain comprising SEQ ID NO:32. In other embodiments, an antibody or antigen-binding fragment comprises SEQ ID NO:1.

In another aspect, the present invention provides diagnostic methods and compositions useful for identifying a specific BCR sequence that is found in T1D, but not healthy controls. In certain embodiments, a PCR probe can be used to identify at-risk individuals.

In another aspect, the present invention provides biologics as therapeutic modalities that target X-cells bearing the T1D-associated BCR and free-floating Abs of the same sequence to prevent the development of T1D and to treat individuals at early stages of T1D slow or reverse disease progression. In particular embodiments, the present invention provides anti-idiotypic antibodies against x-mAb that can be used to detect and eliminate a unique population of pathogenic lymphocytes and thus, be used in the prevention of T1D in at-risk persons or subject with established T1D who may benefit from this treatment, and in those who receive islet replacement or regeneration therapy.

### I. Detection of X-Cells

The present invention provides methods for detecting x-cells in a biological sample including, but not limited to, peripheral blood mononuclear cells (PBMCs). X-cells were identified based on expression of T cell receptor (TCR) and B-cell receptor. X-cells comprise the x-Id (SEQ ID NO:1). In certain embodiments, x-cells comprise VH comprising SEQ ID NOS:1, 44 and 46, as well as VL comprising SEQ ID NOS:38, 40 and 42. X-cells comprise a VH as set forth in SEQ ID NO:28 and a VL as set forth in SEQ ID NO:30 or 32. X-cells can further comprise TCR alpha comprising SEQ ID NOS:48, 50 and 52, as well as TCR beta comprising SEQ ID NOS:54, 56 and 58. In certain embodiments, TCR alpha comprises SEQ ID NO:33 and TCR beta comprises SEQ ID NO:34. The presence of both BCR and TCR molecules on the same cells can distinguish X-cells from conventional T cells which express only the TCR and B-cells which express only BCR. In particular embodiments, the analysis is performed via multicolor flow cytometer, flow cytometric imaging using an AMNIS machine, and at the single cell level using single cell RNA-seq (scRNA-seq).

For flow cytometry and AMNIS, the method comprises a cocktail of fluorochrome-conjugated monoclonal antibodies against TCR, IgD, CD19, CD5 to identify and distinguish X-cells from conventional B and T cells. Additionally, antibodies against IgM, IgG and IgA and surface costimulatory molecules are as described herein.

For scRNA-seq, single X-cells are sorted based on their surface phenotype as described above and subject for transcriptome analysis as compared to T and B-cells. In specific embodiments, FlowJO is used to analyze presence, frequency and phenotype of X-cells.

A. Preparation of PBMCs

In a specific embodiment, single cell PBMC suspension can be prepared as follows:
1. Dilute blood sample at least 1:1 with PBS in a conical tube.
2. Underlay the diluted sample with a volume of Ficoll that is equal to the original sample volume.
3. Centrifuge at 400×g for 20 minutes at room temperature with the brake OFF.
4. Harvest PBMC located at the interface of the PBS and Ficoll layers into a fresh tube.
5. Fill the tube with PBS to wash the cells.
6. Centrifuge the cells at 300-400×g for 4-5 minutes at 2-8° C. Discard supernatant.
7. Resuspend the cell pellet in an appropriate volume of Flow Cytometry Staining Buffer or buffer of choice and perform a cell count and viability analysis.
8. Centrifuge cells as in Step 4 and resuspend in appropriate volume of Flow Cytometry Staining Buffer or buffer of choice so that the final cell concentration is 1×10⁷ cells/mL.

B. Detection and Phenotyping of X-Cells

In another embodiment, X-cells can be detected and phenotyped as follows:
1. Distribute 100-uL aliquots of the cell suspension (10⁶ cells) to tubes.
2. (optional) To block nonspecific Fc-mediated interaction, add 2.5 ug of Fc Block per PBMC per tube and incubate for 10 minutes at room temperature.
3. Add predetermined optimal concentrations of indicated fluorochrome-conjugated antibodies (APC-CD5, BV421-CD19, FITC-TCR, PE-IgD) to cells and incubate for 20 minutes on ice protect from light.
4. Wash the cells two times with 2-mL (for tubes) volumes of Stain Buffer. Centrifuge cells at 300 g for 5 minutes.
5. Carefully aspirate (tubes) or invert and blot away (for tubes) supernatants from cell pellets.
6. Tap tubes to loosen the cell pellet.
7. Resuspend the cell pellet in 0.5-mL (for tubes) volumes of Stain Buffer.
8. Analyze stained cell samples by flow cytometry.
9. Acquired samples (5×10⁵ to 1×10⁶ live events) were properly compensated using single color stains. Data analysis, gating and graphical presentation were done using FlowJo software (TreeStar). Doublets were excluded from analysis using SSC-Height versus SSC-Width and FSC-Height versus FSC-width plots.
10. Three types of specificity controls were used. First, compensations were properly set using single-color stained samples. Fluorescence-Minus One (FMO) and isotype controls were used to properly gate positive cells and set up quadrants.
11. Third, comparison of positive and/or negative cells [conventional B-cells (B_{con}) and/or T cells (T_{con})] in samples were used as internal controls.
12. When appropriate, stimulated vs unstimulated samples provided additional biological controls.

C. Gating Strategy

In specific embodiments, the gating strategy comprises:
1. Lymphocytes were firstly gated strictly using FSC/SSC parameters followed by doublet exclusion using above state method.
2. Gated lymphocyte was separated in three different T cell populations based on CD5 and CD19 expression.
   (i) CD5−CD19+ (B_{con} cells)
   (ii) CD5+CD19+ (CD5+ B-cells)
   (iii) CD+CD19− (Tcon cells)
3. The results show that lymphocytes coexpressing BCR and TCR are found predominantly among CD5+CD19⁻population. To identify these new (DE cells) dual expressor cells, we gated on CD5+CD19+ cells subset and look for expression of TCR and IgD.
4. You can find predominantly among CD5+CD19⁻ population, with the majority expressing IgD, phenotypically DE cells are identified as CD5+CD19+TCR+IgD+ cells and this major cells subset is referred to as IgD+ DE cells.
5. Some DE cells, however, are IgD− (CD5+CD19+TCR) and this minor cell subset is referred to as IgD− DE cells.
6. IgD+ DE cells also expressed IgM (IgD+IgM+), whereas IgD− DE cells included IgG+ cells, suggesting the IgD+ subset represents class-switched DE cells.

### II. In Vitro Expansion of X-Cells

In a further embodiment, the present invention provides a method for in vitro expansion of X-cells. The method includes using high-speed sorting flow cytometry and use of surface staining and sorting X-cells in sterile tubes and culture in complete tissue cultures supplemented with growth factors.

III. Production of Recombinant and Naturally-Produced x-mAb

The present invention also provides a method for production of recombinant and naturally-produced x-mAb. The method comprises sorting of X-cells as described above, and immortalizing them using Epstein-Barr Virus (EBV). Immortalized X-cells spontaneously secrete x-mAb of IgM isotype. Secreted antibodies are characterized using SDS page and their isotype identified using commercially available kits. Commercially available kits can be used to purify secreted antibodies.

x-mAb has been generated by cloning the light and heavy chains from single X-cells and expression into IgG Vector. The vectors expressing light and heavy chains are used to co-transfect 293A cells that secrete x-mAb bearing the IgG isotype. Secreted antibodies are characterized using SDS page and their isotype identified using commercially available kits.

IV. Use of x-mAb to Stimulate and Detect Islet-Reactive T Cells

In further embodiments, the present invention provides methods for the use of x-mAb to stimulate and detect islet-reactive T cells. x-mAb can be used to stimulate CD4 T cells in 24-well plates. x-mAb is added to PBMCs and T cell activation is examined by measuring upregulation of surface antigen CD69 and proliferation, which is measured by using CFSE dilution assay.

The ability of x-mAb to identify insulin autoantigen reactive T cells can be determined by its ability to block binding insulin-HLA-DQ8 tetramers. In addition, x-mAb stimulation of PBMCs from T1D patients leads to expansion of insulin-reactive T cells in vitro. This is determined by using insulin-HLA-DQ tetramers.

In certain embodiments, x-mAb is used to detect specific T cells by using flow cytometry. X-cells are used to stain PBMCs and secondary anti-human immunoglobulin antibody is used to detect T cells that are recognized by x-mAb.

### V. Genotyping HLA-DQ for Determining Risk of T1D

The present invention also provides a method for genotyping of HLA-DQ for determining risk of T1D for individuals bearing x-mAb. In a specific embodiment, a PCR probe is used to identify at-risk individuals since the x-mAb is present in individuals carrying the HLA-allele, which differs from HLA-DQ8 that predisposes at single amino acid in the beta chain of position of 57 of beta chain. Individuals carrying DQ7, which is not associated with T1D and has an aspartic acid at this position, whereas individuals carrying the predisposing DQ8 allele has non-Aspartic acid at this position. Therefore, screening for risk of T1D would involve genotype HLA molecules in individuals who are positive for the x-mAb.

### VI. Screening for the Presence of the X-Clonotype

In an alternative embodiment, the present invention provides a method for using a PCR probe to screen peripheral blood for the presence of the X-clonotype. In one embodiment, the method comprises extraction of RNA from PMBCs using standard methods and use of RT-PCR to convert RNA into cDNA using commercially available kits.

To detect the clonotype of X-cells associated with type 1 diabetes, primers are used to amplify the heavy chain, if present, using PCR. The PCR products were amplified and a band size of 400b was visualized on 1.2% Agarose gel.

To confirm the presence of specific X-cell clonotype, the excised band was sequenced and its identity confirmed using in house analysis software and the National Center for Biotechnology Information IgBlast server or the Immunogenetics server.

A. Reverse Transcription (RT)-PCR

For RT-PCR, RNA from fresh PBMCs was extracted using the RNeasy blood mini kit (Quigen) according to the manufacturer's instructions, followed by NanoDrop (ND-1000 spectrophotometer) measurement for concentration and purity. Reverse transcription (RT) PCR was performed on approximately 1 μg of purified RNA to prepare cDNA by using the RevertAid First Strand cDNA Synthesis Kit (Thermofisher) as per the kit protocol. Briefly, RNA was incubated with 0.5× reaction mix, random hexamer primer, and RevertAid M-MuLV RT (200 U/μL) enzyme mix in a final volume of 20 μL at 25° C. for 10 min, followed by 42° C. for 60 mm and inactivation at 70° C. for 5 min. Positive (GAPDH specific primers) and negative control (reaction mixture without RT enzymes) reactions were used to verify the results of the cDNA synthesis steps.

B. Primer Design and PCR Reaction

For primer design and PCR reaction, cDNA (2 μL) synthesized by RT PCR was then used for PCR amplification in a total volume of 25 μL with 2× QIAGEN HotStarTaq master mix. To detect the target clonotype using PCR amplification, the present inventors designed VH_{04-b} specific leader sense primer that were paired with antisense primers complementary to specific different junctional regions matching to, DH₀₅₋₀₁₈ and JH_{04-01*02}, including N1 and N2 nucleotide addition. These primers included the following:

Primers N1-D-N2
(SEQ ID NO: 17)
VH4 sense primer, 5-GCTGGAGTGGATTGGGAGTA-3
(SEQ ID NO: 18)
VDJ antisense primer, 5′CCCAGTAGTCAAAGTAG
TAAACCATA3′
Primers N2-J
(SEQ ID NO: 17)
VH4 sense primer, 5-GCTGGAGTGGATTGGGAGTA-3
(SEQ ID NO: 19)
VDJ antisense primer, 5′TCCCTGGCCCCAGTAGT
CAAAGTAGTA 3′
Other primers include SEQ ID NOS: 23-24.

The PCR amplification was performed using a thermocycler (BioRad T100) under the following conditions: initial denaturation at 95° C. for 3 min, 95° C. for 30 s, 54° C. for 30 s, 40 cycles at 72° C. for 1 min, and 72° C. for 60 s followed by a final extension step at 72° C. for 10 min. The PCR products were amplified and a band size of 400 b was visualized on 1.2% Agarose gel.

For Sanger sequencing, 400 bp Amplicons were directly excised from the agarose Purification of product was performed using PCR purification kit (Quigen) and samples were sent to Sanger sequencing at the Johns Hopkins Medical Institute GRCF sequencing core facility to verify the sequences. Sequences were analyzed using in house analysis software and the National Center for Biotechnology Information IgBlast server or the Immunogenetics server.

### VII. Anti-Idiotypic Antibodies for Therapy

In a specific embodiment, the present invention provides an isolated antibody or antibody-binding fragment thereof that specifically binds to x-Id, wherein the antibody or antibody-binding fragment comprises heavy chain complementarity determining regions (CDRs) 1, 2 and 3. In a further embodiment, the isolated antibody further comprises light chain CDRs 1, 2 and 3.

The present invention also provides an isolated antibody or antigen-binding fragment thereof that specifically binds to x-Id, wherein the antibody or antigen-binding fragment thereof comprises (a) a heavy chain variable region (VH) comprising CDR1, CDR2, and CDR; and (b) a light chain variable region (VL) comprising CDR1, CDR2, and CDR3.

In particular embodiments, the isolated antibody or antigen-binding fragment described herein is an antagonist of x-Id activity.

The present invention also provides an isolated nucleic acid molecule encoding the anti-x-Id antibody or antigen-binding fragment thereof. In a specific embodiment, a vector comprises a nucleic acid molecule described herein. In another embodiment, a host cell comprises a vector described herein. The host cell can be a prokaryotic or a eukaryotic cell.

In particular embodiments, the present invention provides a method for producing an anti-x-Id antibody or antigen-binding fragment thereof comprising the steps of (a) culturing a host cell under conditions suitable for expression of the x-Id antibody or antigen-binding fragment thereof by the host cells; and (b) recovering the x-Id antibody or antigen-binding fragment thereof.

The present invention also provides a composition comprising an anti-x-Id antibody or antigen-binding fragment thereof and a suitable pharmaceutical carrier. In particular embodiments, the composition is formulated for intravenous, intramuscular, oral, subcutaneous, intraperitoneal, intrathecal or intramuscular administration. The anti-idiotypic antibodies of the present invention can be conjugated with a therapeutic agent including, but not limited to, a toxin.

In another aspect, the present invention provides methods of treatment. In certain embodiments, a method for treating diabetes in a mammal comprises the step of administering to the mammal a therapeutically effective amount of the antibody or antigen-binding fragment thereof that specifically binds to x-Id. In one embodiment, a method for treating or preventing type 1 diabetes (T1D) in a subject having T1D or a risk thereof comprises the step of administering to the patient a therapeutically effective amount of an antibody or antigen-binding fragment described herein. In further embodiments, the antigen-binding fragment is selected from the group consisting of an scFv, sc(Fv)2, Fab, F(ab)2, and a diabody.

In a specific embodiment, the present invention provides an antibody or antigen-binding fragment thereof that specifically binds SEQ ID NO:1. In another embodiment, the present invention provides an antibody or antigen-binding fragment thereof that specifically binds (i) a B-cell receptor expressed on a lymphocyte, wherein the B-cell receptor comprises SEQ ID NO:1; or (ii) a free-floating antibody comprising SEQ ID NO:1. In a further embodiment, an antibody or antigen-binding fragment thereof that specifically binds an antibody comprising SEQ ID NO:1. In certain embodiments, the antibody or antigen-binding fragment prevents or reduces the binding of antigen to SEQ ID NO:1. Furthermore, the antigen-binding fragment can comprise an scFv, sc(Fv)2, Fab, F(ab)2, and a diabody.

In further embodiments, the compositions and methods of the present invention can be utilized to detect, diagnose, and/or assess the risk of other autoimmune diseases. In specific embodiments, the autoimmune diseases comprises ankylosing spondylitis, chronic inflammatory demyelinating polyneuropathy (CIDP), Crohn's disease, dermatomyositis, Graves' disease, Guillain-Barre syndrome, systemic lupus erythematosus, multiple sclerosis, myasthenia gravis, polyarteritis nodosa, primary biliary cirrhosis, psoriatic arthritis, rheumatoid arthritis, scleroderma or ulcerative colitis. In other embodiments, the present invention can be utilized to address rare autoimmune diseases like IgG4-related diseases and pemphigus vulgaris.

In further embodiments, the autoimmune disease can include, but is not limited to, achalasia, Addison's disease, adult Still's disease, agammaglobulinemia, alopecia areata, amyloidosis, ankylosing spondylitis, anti-GBM/anti-TBM nephritis, antiphospholipid syndrome, autoimmune angioedema, autoimmune dysautonomia, autoimmune encephalomyelitis, autoimmune hepatitis, autoimmune inner ear disease, autoimmune myocarditis, autoimmune oophoritis, autoimmune orchitis, autoimmune pancreatitis, autoimmune retinopathy, Balo disease, Behcet's disease, benign mucosal pemphigoid, bullous pemphigoid, Castleman disease, Chagas disease, chronic inflammatory demyelinating polyneuropathy, chronic recurrent multifocal osteomyelitis, Churg-Strauss syndrome, cicatricial pemphigoid, coeliac disease, Cogan's syndrome, cold agglutinin disease, congenital heart block, Coxsackie myocarditis, CREST syndrome, dermatitis herpetiformis, Devic's disease, discoid lupus, Dressier's syndrome, endometriosis, eosinophilic esophagitis, eosinophilic fasciitis, erythema nodosum, essential mixed cryoglobulinemia, Evans syndrome, fibromyalgia, fibrosing alveolitis, giant cell arteritis, giant cell myocarditis, glomerulonephritis, Goodpasture's syndrome, granulomatosis with polyangiitis, Grave's disease, Guillain-Barre syndrome, haemolytic anaemia, Hashimoto's disease, Henoch-Schonlein purpura, herpes gestationis, hidradenitis suppurativa, hypogammaglobulinemia, idiopathic thrombocytopenic purpura, IgA nephropathy, IgG4-related sclerosing disease, immune thrombocytopenic purpura, inclusion body myositis, inflammatory bowel diseases, inflammatory myopathies, interstitial cystitis, juvenile arthritis, juvenile myositis, Kawasaki disease, Lambert-Eaton syndrome, leukocytoclastic vasculitis, lichen planus, lichen sclerosus, ligneous conjunctivitis, linear IgA disease, lupus, Lyme disease chronic, Meniere's disease, microscopic polyangiitis, mixed connective tissue disease, Mooren's ulcer, Mucha-Habermann disease, multifocal motor neuropathy, multiple sclerosis, myasthenia gravis, myositis, narcolepsy, neonatal lupus, neuromyelitis optica, neutropenia, ocular cicatricial pemphigoid, optic neuritis, palindromic rheumatism, paraneoplastic cerebellar degeneration, paroxysmal nocturnal hemoglobinuria, Parry Romberg syndrome, pars planitis, Parsonnage-Turner syndrome, pediatric autoimmune neuropsychiatry disorders associated with streptococcal infections (PANDAS), pemphigus, peripheral neuropathy, perivenous encephalomyelitis, pernicious anemia, POEMS syndrome, polyarteritis nodosa, polyglandular syndromes, polymyalgia rheumatica, polymyositis, postmyocardial infarction syndrome, postpericardiotomy syndrome, primary biliary cirrhosis, primary sclerosing cholangitis, progesterone dermatitis, psoriasis, psoriatic arthritis, pure red cell aplasia, pyoderma gangrenosum, Raynaud's phenomenon, reactive arthritis, reflex sympathetic dystrophy, relapsing polychondritis, restless legs syndrome, retroperitoneal fibrosis, rheumatic fever, rheumatoid arthritis, sarcoidosis, Schmidt syndrome, scleritis, scleroderma, Sjogren's syndrome, sperm and testicular autoimmunity, stiff person syndrome, subacute bacterial endocarditis, Sucac's syndrome sympathetic ophtalmia, systemic lupus erythematosus, Takayasu's arteritis, temporal arteritis, Tolosa-Hunt syndrome, transverse myelitis, type 1 diabetes, undifferentiated connective tissue disease, uveitis, vasculitis Vogt-Koyanagi-Harada disease, vitiligo and Wegener's granulomatosis.

A. Definitions

The term “antibody” means an immunoglobulin molecule that recognizes and specifically binds to a target, such as a protein (e.g., the x-Id, a subunit thereof, or the receptor complex), polypeptide, peptide, carbohydrate, polynucleotide, lipid, or combinations of the foregoing through at least one antigen recognition site within the variable region of the immunoglobulin molecule. A typical antibody comprises at least two heavy (HC) chains and two light (LC) chains interconnected by disulfide bonds. Each heavy chain is comprised of a “heavy chain variable region” or “heavy chain variable domain” (abbreviated herein as VH) and a heavy chain constant region. The heavy chain constant region is comprised of three domains, CHI, CH2, and CH3. Each light chain is comprised of a “light chain variable region” or “light chain variable domain” (abbreviated herein as VL) and a light chain constant region. The light chain constant region is comprised of one domain, CI. The VH and VL regions can be further subdivided into regions of hypervariablity, termed Complementarity Determining Regions (CDR), interspersed with regions that are more conserved, termed framework regions (FRs). Each VH and VL region is composed of three CDRs and four FRs, arranged from amino-terminus to carboxy-terminus in the following order: FRI, CDRI, FR2, CDR2, FR3, CDR3, FR4. The variable regions of the heavy and light chains contain a binding domain that interacts with an antigen. As used herein, the term “antibody” encompasses intact poly clonal antibodies, intact monoclonal antibodies, antibody fragments (such as Fab, Fab′, F(ab′)2, Fd, Facb, and Fv fragments), single chain Fv (scFv), minibodies (e.g., sc(Fv)2, diabody), multispecific antibodies such as bispecific antibodies generated from at least two intact antibodies, chimeric antibodies, humanized antibodies, human antibodies, fusion proteins comprising an antigen determination portion of an antibody, and any other modified immunoglobulin molecule comprising an antigen recognition site so long as the antibodies exhibit the desired biological activity. Thus, the term “antibody” includes whole antibodies and any antigen-binding fragment or single chains thereof. Antibodies can be naked or conjugated to other molecules such as toxins, radioisotopes, small molecule drugs, polypeptides, etc.

The term “isolated antibody” refers to an antibody that has been identified and separated and/or recovered from a component of its natural environment. Contaminant components of its natural environment are materials which would interfere with diagnostic or therapeutic uses for the antibody, and may include enzymes, hormones, and other proteinaceous or nonproteinaceous solutes. In some embodiments, the antibody is purified (1) to greater than 95% by weight of antibody as determined by, for example, the Lowry method, and including more than 99% by weight, (2) to a degree sufficient to obtain at least 15 residues of N-terminal or internal amino acid sequence by use of a spinning cup sequenator, or (3) to homogeneity by SDS-PAGE under reducing or non-reducing conditions using Coomassie blue or silver stain. An isolated antibody includes the antibody in situ within recombinant cells since at least one component of the antibody's natural environment will not be present. Ordinarily, however, isolated antibody will be prepared by at least one purification step.

The term “humanized” immunoglobulin refers to an immunoglobulin comprising a human framework region and one or more CDRs from a non-human (usually a mouse or rat) immunoglobulin. The non-human immunoglobulin providing the CDRs is called the “donor” and the human immunoglobulin providing the framework is called the “acceptor.” Constant regions need not be present, but if they are, they must be substantially identical to human immunoglobulin constant regions, i.e., at least about 85-90%, preferably about 95% or more identical. Hence, all parts of a humanized immunoglobulin, except possibly the CDRs, are substantially identical to corresponding parts of natural human immunoglobulin sequences. A “humanized antibody” is an antibody comprising a humanized light chain and a humanized heavy chain immunoglobulin. For example, a humanized antibody would not encompass a typical chimeric antibody as defined above, e.g., because the entire variable region of a chimeric antibody is non-human.

The term “antigen binding fragment” refers to a portion of an intact antibody and refers to the antigenic determining variable regions of an intact antibody. It is known in the art that the antigen binding function of an antibody can be performed by fragments of a full-length antibody. Examples of antigen-binding antibody fragments include, but are not limited to Fab, Fab′, F(ab′)2, Facb, Fd, and Fv fragments, linear antibodies, single chain antibodies, and multi-specific antibodies formed from antibody fragments. In some instances, antibody fragments may be prepared by proteolytic digestion of intact or whole antibodies. For example, antibody fragments can be obtained by treating the whole antibody with an enzyme such as papain, pepsin, or plasmin. Papain digestion of whole antibodies produces F(ab)2 or Fab fragments; pepsin digestion of whole antibodies yields F(ab′)2 or Fab′; and plasmin digestion of whole antibodies yields Facb fragments.

The term “Fab” refers to an antibody fragment that is essentially equivalent to that obtained by digestion of immunoglobulin (typically IgG) with the enzyme papain. The heavy chain segment of the Fab fragment is the Fd piece. Such fragments can be enzymatically or chemically produced by fragmentation of an intact antibody, recombinantly produced from a gene encoding the partial antibody sequence, or it can be wholly or partially synthetically produced. The term “F(ab)2” refers to an antibody fragment that is essentially equivalent to a fragment obtained by digestion of an immunoglobulin (typically IgG) with the enzyme pepsin at pH 4.0-4.5. Such fragments can be enzymatically or chemically produced by fragmentation of an intact antibody, recombinantly produced from a gene encoding the partial antibody sequence, or it can be wholly or partially synthetically produced. The term “Fv” refers to an antibody fragment that consists of one NH and one N domain held together by noncovalent interactions.

The terms “x-Id antibody,” “anti-x-Id antibody,” “anti-x-Id,” “antibody that binds to x-Id” and any grammatical variations thereof refer to an antibody that is capable of specifically binding to x-Id with sufficient affinity such that the antibody is useful as a therapeutic agent or diagnostic reagent in targeting x-Id. The extent of binding of an anti-x-Id antibody disclosed herein to an unrelated, non-x-Id protein is less than about 10% of the binding of the antibody to x-Id as measured, e.g., by a radioimmunoassay (RIA), BIACORE™ (using recombinant x-Id as the analyte and antibody as the ligand, or vice versa), or other binding assays known in the art. In certain embodiments, an antibody that binds to x-Id has a dissociation constant (KD) of <1 μM, <100 nM, <50 nM, <10 nM, or <1 nM.

The term “% identical” between two polypeptide (or polynucleotide) sequences refers to the number of identical matched positions shared by the sequences over a comparison window, taking into account additions or deletions (i.e., gaps) that must be introduced for optimal alignment of the two sequences. A matched position is any position where an identical nucleotide or amino acid is presented in both the target and reference sequence. Gaps presented in the target sequence are not counted since gaps are not nucleotides or amino acids. Likewise, gaps presented in the reference sequence are not counted since target sequence nucleotides or amino acids are counted, not nucleotides or amino acids from the reference sequence. The percentage of sequence identity is calculated by determining the number of positions at which the identical amino acid residue or nucleic acid base occurs in both sequences to yield the number of matched positions, dividing the number of matched positions by the total number of positions in the window of comparison and multiplying the result by 100 to yield the percentage of sequence identity. The comparison of sequences and determination of percent sequence identity between two sequences can be accomplished using readily available software both for online use and for download. Suitable software programs are available from various sources, and for alignment of both protein and nucleotide sequences. One suitable program to determine percent sequence identity is bl2seq, part of the BLAST suite of program available from the U.S. government's National Center for Biotechnology Information BLAST web site. Bl2seq performs a comparison between two sequences using either the BLASTN or BLASTP algorithm. BLASTN is used to compare nucleic acid sequences, while BLASTP is used to compare amino acid sequences. Other suitable programs are, e.g., Needle, Stretcher, Water, or Matcher, part of the EMBOSS suite of bioinformatics programs and also available from the European Bioinformatics Institute (EBI) at www.ebi.ac.uk/Tools/psa. In certain embodiments, the percentage identity “X” of a first amino acid sequence to a second sequence amino acid is calculated as 100×(Y/Z), where Y is the number of amino acid residues scored as identical matches in the alignment of the first and second sequences (as aligned by visual inspection or a particular sequence alignment program) and Z is the total number of residues in the second sequence. If the length of a first sequence is longer than the second sequence, the percent identity of the first sequence to the second sequence will be higher than the percent identity of the second sequence to the first sequence. One skilled in the art will appreciate that the generation of a sequence alignment for the calculation of a percent sequence identity is not limited to binary sequence-sequence comparisons exclusively driven by primary sequence data. Sequence alignments can be derived from multiple sequence alignments. One suitable program to generate multiple sequence alignments is ClustalW2 (ClustalX is a version of the ClustalW2 program ported to the Windows environment). Another suitable program is MUSCLE. ClustalW2 and MUSCLE are alternatively available, e.g., from the European Bioinformatics Institute (EBI).

The term “therapeutic agent” refers to any biological or chemical agent used in the treatment of a disease or disorder. Therapeutic agents include any suitable biologically active chemical compounds, biologically derived components such as cells, peptides, antibodies, and polynucleotides, and radiochemical therapeutic agents such as radioisotopes. In some embodiments, the therapeutic agent comprises a chemotherapeutic agent or an analgesic.

As used herein, the terms “treatment,” “treating,” “treat” and the like, refer to obtaining a desired pharmacologic and/or physiologic effect. The terms are also used in the context of the administration of a “therapeutically effective amount” of an agent, e.g., an anti-x-Id antibody. The effect may be prophylactic in terms of completely or partially preventing a particular outcome, disease or symptom thereof and/or may be therapeutic in terms of a partial or complete cure for a disease and/or adverse effect attributable to the disease. “Treatment,” as used herein, covers any treatment of a disease in a subject, particularly in a human, and includes: (a) preventing the disease from occurring in a subject which may be predisposed to the disease but has not yet been diagnosed as having it; (b) inhibiting the disease, i.e., arresting its development; and (c) relieving the disease, e.g., causing regression of the disease, e.g., to completely or partially remove symptoms of the disease. In particular embodiments, the term is used in the context of preventing or treating any x-Id-mediated disease including diabetes.

B. Anti-x-Id Antibodies

The antibodies or antigen-binding fragment thereof of this disclosure specifically bind to x-Id. In specific embodiments, these antibodies or antigen-binding fragments specifically bind to human x-Id. “Specifically binds” as used herein means that the antibody or antigen-binding fragment preferentially binds x-Id (e.g., human x-Id, mouse x-Id) over other proteins. In certain instances, the anti-x-Id antibodies of the disclosure have a higher affinity for x-Id than for other proteins. Anti-x-Id antibodies that specifically bind x-Id may have a binding affinity for human x-Id of less than or equal to 1×10⁻⁷ M, less than or equal to 2×10⁻⁷ M, less than or equal to 3×10⁻⁷ M, less than or equal to 4×10⁻⁷ M, less than or equal to 5×10⁻⁷ M, less than or equal to 6×10⁻⁷ M, less than or equal to 7×10⁻⁷ M, less than or equal to 8×10⁻⁷ M, less than or equal to 9×10⁻⁷ M, less than or equal to 1×10⁻⁸ M, less than or equal to 2×10⁻⁸ M, less than or equal to 3×10⁻⁸ M, less than or equal to 4×10⁻⁸ M, less than or equal to 5×10⁻⁸M, less than or equal to 6×10⁻⁸ M, less than or equal to 7×10⁻⁸ M, less than or equal to 8×10⁻⁸M, less than or equal to 9×10⁻⁸M, less than or equal to 1×10⁻⁹ M, less than or equal to 2×10⁻⁹ M, less than or equal to 3×10⁻⁹ M, less than or equal to 4×10⁻⁹ M, less than or equal to 5×10⁻⁹M, less than or equal to 6×10⁻⁹ M, less than or equal to 7×10⁻⁹ M, less than or equal to 8×10⁻⁹M, less than or equal to 9×10⁻⁹ M, less than or equal to 1×10⁻¹⁰ M, less than or equal to 2×10⁻¹⁰ M, less than or equal to 3×10⁻¹⁰ M, less than or equal to 4×10⁻¹⁰ M, less than or equal to 5×10⁻¹⁰ M, less than or equal to 6×10⁻¹⁰ M, less than or equal to 7×10⁻¹⁰ M, less than or equal to 8×10⁻¹⁰ M, less than or equal to 9×10⁻¹⁰ M, less than or equal to 1×10⁻¹¹M, less than or equal to 2×10⁻¹¹ M, less than or equal to 3×10⁻¹¹ M, less than or equal to 4×10⁻¹¹ M, less than or equal to 5×10⁻¹¹ M, less than or equal to 6×10⁻¹¹ M, less than or equal to 7×10⁻¹¹ M, less than or equal to 8×10⁻¹¹ M, less than or equal to 9×10⁻¹¹ M, less than or equal to 1×10⁻¹² M, less than or equal to 2×10⁻¹² M, less than or equal to 3×10⁻¹² M, less than or equal to 4×10⁻¹² M, less than or equal to 5×10⁻¹² M, less than or equal to 6×10⁻¹² M, less than or equal to 7×10⁻¹² M, less than or equal to 8×10⁻¹² M, or less than or equal to 9×10⁻¹² M. Methods of measuring the binding affinity of an antibody are well known in the art and include Surface Plasmon Resonance (SPR) (Morton and Myszka “Kinetic analysis of macromolecular interactions using surface plasmon resonance biosensors” Methods in Enzymology (1998) 295, 268-294), Bio-Layer Interferometry, (Abdiche et al “Determining Kinetics and Affinities of Protein Interactions Using a Parallel Real-time Label-free Biosensor, the Octet” Analytical Biochemistry (2008) 377, 209-217), Kinetic Exclusion Assay (KinExA) (Darling and Brault “Kinetic exclusion assay technology: characterization of molecular interactions” Assay and Drug Dev Tech (2004) 2, 647-657), isothermal calorimetry (Pierce et al “Isothermal Titration calorimetry of Protein-Protein Interactions” Methods (1999) 19, 213-221) and analytical ultracentrifugation (Lebowitz et al “Modem analytical ultracentrifugation in protein science: A tutorial review” Protein Science (2002), 11:2067-2079).

1. Antibody Fragments

The present disclosure encompasses the antibody fragments or domains described herein that retains the ability to specifically bind to x-Id (e.g., human x-Id). Antibody fragments include, e.g., Fab, Fab′, F(ab′)2, Facb, and Fv. These fragments may be humanized or fully human. Antibody fragments may be prepared by proteolytic digestion of intact antibodies. For example, antibody fragments can be obtained by treating the whole antibody with an enzyme such as papain, pepsin, or plasmin. Papain digestion of whole antibodies produces F(ab)2 or Fab fragments; pepsin digestion of whole antibodies yields F(ab′)2 or Fab′; and plasmin digestion of whole antibodies yields Facb fragments.

Alternatively, antibody fragments can be produced recombinantly. For example, nucleic acids encoding the antibody fragments of interest can be constructed, introduced into an expression vector, and expressed in suitable host cells. See, e.g., Co, M. S. et al., J. Immunol., 152:2968-2976 (1994); Better, M. and Horwitz, A. H., Methods in Enzymology, 178:476-496 (1989); Pluckthun, A and Skerra, A, Methods in Enzymology, 178:476-496 (1989); Lamoyi, E., Methods in Enzymology, 121:652-663 (1989); Rousseaux, J. et al., Methods in Enzymology, (1989) 121:663-669 (1989); and Bird, R E. et al., TIBTECH, 9:132-137 (1991)). Antibody fragments can be expressed in and secreted from E. coli, thus allowing the facile production of large amounts of these fragments. Antibody fragments can be isolated from the antibody phage libraries. Alternatively, Fab′-SH fragments can be directly recovered from E. coli and chemically coupled to form F(ab)2 fragments (Carter et al., Bio/Technology, 10:163-167 (1992)). According to another approach, F(ab′)2 fragments can be isolated directly from recombinant host cell culture. Fab and F(ab′) 2 fragment with increased in vivo half-life comprising a salvage receptor binding epitope residues are described in U.S. Pat. No. 5,869,046.

2. Minibodies

Also encompassed are minibodies of the antibodies described herein. Minibodies of anti-x-Id antibodies include diabodies, single chain (scFv), and single-chain (Fv)2 (sc(Fv)2).

A “diabody” is a bivalent minibody constructed by gene fusion (see, e.g., Holliger, P. et al., Proc. Natl. Acad. Sci. USA., 90:6444-6448 (1993); EP 404,097; WO 93/11161). Diabodies are dimers composed of two polypeptide chains. The VL and VH domain of each polypeptide chain of the diabody are bound by linkers. The number of amino acid residues that constitute a linker can be between 2 to 12 residues (e.g., 3-10 residues or five or about five residues). The linkers of the polypeptides in a diabody are typically too short to allow the VL and VH to bind to each other. Thus, the VL and VH encoded in the same polypeptide chain cannot form a single-chain variable region fragment, but instead form a dimer with a different single-chain variable region fragment. As a result, a diabody has two antigen-binding sites.

An scFv is a single-chain polypeptide antibody obtained by linking the VH and VL with a linker (see e.g., Huston et al., Proc. Natl. Acad. Sci. US.A., 85:5879-5883 (1988); and Pluckthun, “The Pharmacology of Monoclonal Antibodies” Vol. 113, Ed Resenburg and Moore, Springer Verlag, New York, pp. 269-315, (1994)). Each variable domain (or a portion thereof) is derived from the same or different antibodies. Single chain Fv molecules preferably comprise an scFv linker interposed between the VH domain and the VL domain. Exemplary scFv molecules are known in the art and are described, for example, in U.S. Pat. No. 5,892,019; Ho et al, Gene, 77:51 (1989); Bird et al., Science, 242:423 (1988); Pantoliano et al, Biochemistry, 30: 101 17 (1991); Milenic et al, Cancer Research, 51:6363 (1991); Takkinen et al, Protein Engineering, 4:837 (1991).

The term “scFv linker” as used herein refers to a moiety interposed between the VL and VH domains of the scFv. The scFv linkers preferably maintain the scFv molecule in an antigen-binding conformation. In one embodiment, an scFv linker comprises or consists of an scFv linker peptide. In certain embodiments, an scFv linker peptide comprises or consists of a Gly-Ser peptide linker. In other embodiments, an scFv linker comprises a disulfide bond.

The order of VHs and VLs to be linked is not particularly limited, and they may be arranged in any order. Examples of arrangements include: [VH] linker [VL]; or [VL] linker [VH]. The H chain V region and L chain V region in an scFv may be derived from any anti-x-Id antibody or antigen-binding fragment thereof described herein.

An sc(Fv)2 is a minibody in which two VHs and two VLs are linked by a linker to form a single chain (Hudson, et al., J Immunol. Methods, (1999) 231: 177-189 (1999)). An sc(Fv)2 can be prepared, for example, by connecting scFvs with a linker. The sc(Fv)2 of the present invention include antibodies preferably in which two VHs and two VLs are arranged in the order of: VH, VL, VH, and VL ([VH] linker [VL] linker [VH] linker [VL]), beginning from the N terminus of a single-chain polypeptide; however, the order of the two VHs and two VLs is not limited to the above arrangement, and they may be arranged in any order. Examples of arrangements are listed below:
[VL] linker [VH] linker [VH] linker [VL]
[VH] linker [VL] linker [VL] linker [VH]
[VH] linker [VH] linker [VL] linker [VL]
[VL] linker [VL] linker [VH] linker [VH]
[VL] linker [VH] linker [VL] linker [VH]

Normally, three linkers are required when four antibody variable regions are linked; the linkers used may be identical or different. There is no particular limitation on the linkers that link the VH and VL regions of the minibodies. In some embodiments, the linker is a peptide linker. Any arbitrary single-chain peptide comprising about 3 to 25 residues (e.g., 5, 6, 7, 8, 9, 10, 11, 12, 13, 14, 15, 16, 17, 18) can be used as a linker.

In other embodiments, the linker is a synthetic compound linker (chemical cross-linking agent). Examples of cross-linking agents that are available on the market include N-hydroxysuccinimide (NHS), disuccinimidylsuberate (DSS), bis(sulfosuccinimidyl)suberate (BS3), dithiobis(succinimidy Ipropionate) (DSP), dithiobis(sulfosuccinimidy Ipropionate) (DTSSP), ethyleneglycol bis(succinimidylsuccinate) (EGS), ethyleneglycol bis(sulfosuccinimidylsuccinate) (sulfo-EGS), disuccinimidyl tartrate (DST), disulfosuccinimidyl tartrate (sulfo-DST), bis[2-(succinimidooxycarbonyloxy)ethyl]sulfone (BSOCOES), and bis[2-(sulfosuccinimidooxycarbonyloxy)ethyl]sulfone (sulfo-BSOCOES).

The amino acid sequence of the VH or VL in the antibody fragments or minibodies may include modifications such as substitutions, deletions, additions, and/or insertions. For example, the modification may be in one or more of the CDRs of the anti-x-Id antibodies described herein. In certain embodiments, the modification involves one, two, or three amino acid substitutions in one, two, or three CDRs of the VH and/or one, two, or three CDRs of the VL domain of the anti-x-Id minibody. Such substitutions are made to improve the binding and/or functional activity of the anti-x-Id minibody. In other embodiments, one, two, or three amino acids of one or more of the six CDRs of the anti-x-Id antibody or antigen-binding fragment thereof may be deleted or added as long as there is x-Id binding and/or functional activity when VH and VL are associated.

3. VHH

VHH also known as nanobodies are derived from the antigen-binding variable heavy chain regions (VHHs) of heavy chain antibodies found in camels and llamas, which lack light chains. The present disclosure encompasses VHHs that specifically bind x-Id.

4. Variable Domain of New Antigen Receptors (VNARs)

A VNAR is a variable domain of a new antigen receptor (IgNAR). IgNARs exist in the sera of sharks as a covalently linked heavy chain homodimer. It exists as a soluble and receptor bound form consisting of a variable domain (VNAR) with differing numbers of constant domains. The VNAR is composed of a CDR1 and CDR3 and in lieu of a CDR2 has HV2 and HV4 domains (see, e.g., Barelle and Porter, Antibodies, 4:240-258 (2015)). The present disclosure encompasses VNARs that specifically bind x-Id.

5. Constant Regions

Antibodies of this disclosure can be whole antibodies or single chain Fc (scFc) and can comprise any constant region known in the art. The light chain constant region can be, for example, a kappa- or lambda-type light chain constant region, e.g., a human kappa or human lambda light chain constant region. The heavy chain constant region can be, e.g., an alpha-, delta-, epsilon-, gamma-, or mu-type heavy chain constant region, e.g., a human alpha-, human delta-, human epsilon-, human gamma-, or human mu-type heavy chain constant region. In certain instances, the anti-x-Id antibody is an IgA antibody, an IgD antibody, an IgE antibody, an IgG1 antibody, an IgG2 antibody, an IgG3 antibody, an IgG4 antibody, or an IgM antibody.

In one embodiment, the light or heavy chain constant region is a fragment, derivative, variant, or mutein of a naturally occurring constant region. In some embodiments, the variable heavy chain of the anti-x-Id antibodies described herein is linked to a heavy chain constant region comprising a CH1 domain and a hinge region. In some embodiments, the variable heavy chain is linked to a heavy chain constant region comprising a CH2 domain. In some embodiments, the variable heavy chain is linked to a heavy chain constant region comprising a CH3 domain. In some embodiments, the variable heavy chain is linked to a heavy chain constant region comprising a CH2 and CH3 domain. In some embodiments, the variable heavy chain is linked to a heavy chain constant region comprising a hinge region, a CH2 and a CH3 domain. The CH1, hinge region, CH2, and/or CH3 can be from an IgG antibody (e.g., IgGI, IgG4). In certain embodiments, the variable heavy chain of an anti-x-Id antibody described herein is linked to a heavy chain constant region comprising a CHI domain, hinge region, and CH2 domain from IgG4 and a CH3 domain from IgGI. In certain embodiments such a chimeric antibody may contain one or more additional mutations in the heavy chain constant region that increase the stability of the chimeric antibody. In certain embodiments, the heavy chain constant region includes substitutions that modify the properties of the antibody.

In certain embodiments, an anti-x-Id antibody of this disclosure is an IgG isotype antibody. In one embodiment, the antibody is IgG1. In another embodiment, the antibody is IgG2. In yet another embodiment, the antibody is IgG4. In some instances, the IgG4 antibody has one or more mutations that reduce or prevent it adopting a functionally monovalent format. For example, the hinge region of IgG4 can be mutated to make it identical in amino acid sequence to the hinge region of human IgG1 (mutation of a serine in human IgG4 hinge to a proline). In some embodiments, the antibody has a chimeric heavy chain constant region (e.g., having the CH1, hinge, and CH2 regions of IgG4 and CH3 region of IgG1).

6. Bispecific Antibodies

In certain embodiments, an anti-x-Id antibody of this disclosure is a bispecific antibody. Bispecific antibodies are antibodies that have binding specificities for at least two different epitopes. Exemplary bispecific antibodies may bind to two different epitopes of the x-Id protein. Other such antibodies may combine an x-Id binding site with a binding site for another protein. Bispecific antibodies can be prepared as full length antibodies or low molecular weight forms thereof (e.g., F(ab′) 2 bispecific antibodies, sc(Fv)2 bispecific antibodies, diabody bispecific antibodies).

Traditional production of full length bispecific antibodies is based on the co-expression of two immunoglobulin heavy chain-light chain pairs, where the two chains have different specificities (Millstein et al., Nature, 305:537-539 (1983)). In a different approach, antibody variable domains with the desired binding specificities are fused to immunoglobulin constant domain sequences. DNAs encoding the immunoglobulin heavy chain fusions and, if desired, the immunoglobulin light chain, are inserted into separate expression vectors, and are co-transfected into a suitable host cell. This provides for greater flexibility in adjusting the proportions of the three polypeptide fragments. It is, however, possible to insert the coding sequences for two or all three polypeptide chains into a single expression vector when the expression of at least two polypeptide chains in equal ratios results in high yields.

According to another approach described in U.S. Pat. No. 5,731,168, the interface between a pair of antibody molecules can be engineered to maximize the percentage of heterodimers that are recovered from recombinant cell culture. The preferred interface comprises at least a part of the CH3 domain. In this method, one or more small amino acid side chains from the interface of the first antibody molecule are replaced with larger side chains (e.g., tyrosine or tryptophan). Compensatory “cavities” of identical or similar size to the large side chain(s) are created on the interface of the second antibody molecule by replacing large amino acid side chains with smaller ones (e.g., alanine or threonine). This provides a mechanism for increasing the yield of the heterodimer over other unwanted end-products such as homodimers.

Bispecific antibodies include cross-linked or “heteroconjugate” antibodies. For example, one of the antibodies in the heteroconjugate can be coupled to avidin, the other to biotin. Heteroconjugate antibodies may be made using any convenient cross-linking methods.

The “diabody” technology provides an alternative mechanism for making bispecific antibody fragments. The fragments comprise a VH connected to a VL by a linker which is too short to allow pairing between the two domains on the same chain. Accordingly, the VH and VL domains of one fragment are forced to pair with the complementary VL and VH domains of another fragment, thereby forming two antigen-binding sites.

7. Conjugated Antibodies

The antibodies or antigen-binding fragments disclosed herein may be conjugated to various molecules including macromolecular substances such as polymers (e.g., polyethylene glycol (PEG), polyethylenimine (PEI) modified with PEG (PEI-PEG), polyglutamic acid (PGA) (N-(2-Hydroxypropyl) methacrylamide (HPMA) copolymers), human serum albumin or a fragment thereof, radioactive materials (e.g., ⁹⁰Y, ¹³¹I), fluorescent substances, luminescent substances, haptens, enzymes, metal chelates, and drugs.

In certain embodiments, an anti-x-Id antibody or antigen-binding fragment thereof is modified with a moiety that improves its stabilization and/or retention in circulation, e.g., in blood, serum, or other tissues, e.g., by at least 1.5, 2, 5, 10, 15, 20, 25, 30, 40, or 50 fold. For example, the anti-x-Id antibody or antigen-binding fragment thereof can be associated with (e.g., conjugated to) a polymer, e.g., a substantially non-antigenic polymer, such as a polyalkylene oxide or a polyethylene oxide. Suitable polymers will vary substantially by weight. Polymers having molecular number average weights ranging from about 200 to about 35,000 Daltons (or about 1,000 to about 15,000, and 2,000 to about 12,500) can be used. For example, the anti-x-Id antibody or antigen-binding fragment thereof can be conjugated to a water soluble polymer, e.g., a hydrophilic polyvinyl polymer, e.g., polyvinylalcohol or polyvinylpyrrolidone. Examples of such polymers include polyalkylene oxide homopolymers such as polyethylene glycol (PEG) or polypropylene glycols, polyoxyethylenated polyols, copolymers thereof and block copolymers thereof, provided that the water solubility of the block copolymers is maintained. Additional useful polymers include polyoxyalkylenes such as polyoxyethylene, polyoxypropylene, and block copolymers of polyoxyethylene and polyoxypropylene; polymethacrylates; carbomers; and branched or unbranched polysaccharides.

The above-described conjugated antibodies or fragments can be prepared by performing chemical modifications on the antibodies or the lower molecular weight forms thereof described herein. Methods for modifying antibodies are well known in the art (e.g., U.S. Pat. Nos. 5,057,313 and 5,156,840).

C. Characterization of Antibodies

The x-Id binding properties of the antibodies described herein may be measured by any standard method, e.g., one or more of the following methods: OCTET®, Surface Plasmon Resonance (SPR), BIACORE™ analysis, Enzyme Linked Immunosorbent Assay (ELISA), EIA (enzyme immunoassay), RIA (radioimmunoassay), and Fluorescence Resonance Energy Transfer (FRET).

The binding interaction of a protein of interest (an anti-x-Id antibody or functional fragment thereof) and a target (e.g., x-Id) can be analyzed using the OCTET® systems. In this method, one of several variations of instruments (e.g., OCTET® QKe and QK), made by the ForteBio company are used to determine protein interactions, binding specificity, and epitope mapping. The OCTET® systems provide an easy way to monitor real-time binding by measuring the changes in polarized light that travels down a custom tip and then back to a sensor.

The binding interaction of a protein of interest (an anti-x-Id antibody or functional fragment thereof) and a target (e.g., x-Id) can be analyzed using Surface Plasmon Resonance (SPR). SPR or Biomolecular Interaction Analysis (BIA) detects biospecific interactions in real time, without labeling any of the interactants.

Changes in the mass at the binding surface (indicative of a binding event) of the BIA chip result in alterations of the refractive index of light near the surface (the optical phenomenon of surface plasmon resonance (SPR)). The changes in the refractivity generate a detectable signal, which is measured as an indication of real-time reactions between biological molecules. Methods for using SPR are described, for example, in U.S. Pat. No. 5,641,640; Raether (1988) Surface Plasmons Springer Verlag; Sjolander and Urbaniczky (1991) Anal. Chem 63:2338-2345; Szabo et al. (1995) Curr. Opin. Struct. Biol. 5:699-705 and on-line resources provide by BIAcore International AB (Uppsala, Sweden). Information from SPR can be used to provide an accurate and quantitative measure of the equilibrium dissociation constant (Kd), and kinetic parameters, including Kon and Koff, for the binding of a biomolecule to a target.

Epitopes can also be directly mapped by assessing the ability of different anti-x-Id antibodies or functional fragments thereof to compete with each other for binding to human x-Id using BIACORE chromatographic techniques (Pharmacia BIAtechnology Handbook, “Epitope Mapping”, Section 6.3.2, (May 1994); see also Johne et al. (1993) J. Immunol. Methods, 160:191-198).

When employing an enzyme immunoassay, a sample containing an antibody, for example, a culture supernatant of antibody-producing cells or a purified antibody is added to an antigen-coated plate. A secondary antibody labeled with an enzyme such as alkaline phosphatase is added, the plate is incubated, and after washing, an enzyme substrate such as p-nitrophenylphosphate is added, and the absorbance is measured to evaluate the antigen binding activity.

Additional general guidance for evaluating antibodies, e.g., Western blots and immunoprecipitation assays, can be found in Antibodies: A Laboratory Manual, ed. by Harlow and Lane, Cold Spring Harbor press (1988)).

D. Affinity Maturation

In one embodiment, an anti-x-Id antibody or antigen-binding fragment thereof is modified, e.g., by mutagenesis, to provide a pool of modified antibodies. The modified antibodies are then evaluated to identify one or more antibodies having altered functional properties (e.g., improved binding, improved stability, reduced antigenicity, or increased stability in vivo). In one implementation, display library technology is used to select or screen the pool of modified antibodies. Higher affinity antibodies are then identified from the second library, e.g., by using higher stringency or more competitive binding and washing conditions. Other screening techniques can also be used. Methods of effecting affinity maturation include random mutagenesis (e.g., Fukuda et al., Nucleic Acids Res., 34:e127 (2006); targeted mutagenesis (e.g., Rajpal et al., Proc. Natl. Acad. Sci. USA, 102:8466-71 (2005); shuffling approaches (e.g., Jermutus et al., Proc. Natl. Acad. Sci. USA, 98:75-80 (2001); and in silica approaches (e.g., Lippow et al., Nat. Biotechnol., 25: 1171-6 (2005).

In some embodiments, the mutagenesis is targeted to regions known or likely to be at the binding interface. If, for example, the identified binding proteins are antibodies, then mutagenesis can be directed to the CDR regions of the heavy or light chains as described herein. Further, mutagenesis can be directed to framework regions near or adjacent to the CDRs, e.g., framework regions, particularly within 10, 5, or 3 amino acids of a CDR junction. In the case of antibodies, mutagenesis can also be limited to one or a few of the CDRs, e.g., to make step-wise improvements.

In one embodiment, mutagenesis is used to make an antibody more similar to one or more germline sequences. One exemplary germlining method can include: identifying one or more germline sequences that are similar (e.g., most similar in a particular database) to the sequence of the isolated antibody. Then mutations (at the amino acid level) can be made in the isolated antibody, either incrementally, in combination, or both. For example, a nucleic acid library that includes sequences encoding some or all possible germline mutations is made. The mutated antibodies are then evaluated, e.g., to identify an antibody that has one or more additional germline residues relative to the isolated antibody and that is still useful (e.g., has a functional activity). In one embodiment, as many germline residues are introduced into an isolated antibody as possible.

In one embodiment, mutagenesis is used to substitute or insert one or more germline residues into a CDR region. For example, the germline CDR residue can be from a germline sequence that is similar (e.g., most similar) to the variable region being modified. After mutagenesis, activity (e.g., binding or other functional activity) of the antibody can be evaluated to determine if the germline residue or residues are tolerated. Similar mutagenesis can be performed in the framework regions.

Selecting a germline sequence can be performed in different ways. For example, a germline sequence can be selected if it meets a predetermined criterion for selectivity or similarity, e.g., at least a certain percentage identity, e.g., at least 75, 80, 85, 90, 91, 92, 93, 94, 95, 96, 97, 98, 99, or 99.5% identity, relative to the donor non-human antibody. The selection can be performed using at least 2, 3, 5, or 10 germline sequences. In the case of CDR1 and CDR2, identifying a similar germline sequence can include selecting one such sequence. In the case of CDR3, identifying a similar germline sequence can include selecting one such sequence, but may include using two germline sequences that separately contribute to the amino-terminal portion and the carboxy-terminal portion. In other implementations, more than one or two germline sequences are used, e.g., to form a consensus sequence.

Calculations of “sequence identity” between two sequences are performed as follows. The sequences are aligned for optimal comparison purposes (e.g., gaps can be introduced in one or both of a first and a second amino acid or nucleic acid sequence for optimal alignment and non-homologous sequences can be disregarded for comparison purposes). The optimal alignment is determined as the best score using the GAP program in the GCG software package with a Blossum 62 scoring matrix with a gap penalty of 12, a gap extend penalty of 4, and a frameshift gap penalty of 5. The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position. The percent identity between the two sequences is a function of the number of identical positions shared by the sequences.

In other embodiments, the antibody may be modified to have an altered glycosylation pattern (i.e., altered from the original or native glycosylation pattern). As used in this context, “altered” means having one or more carbohydrate moieties deleted, and/or having one or more glycosylation sites added to the original antibody. Addition of glycosylation sites to the presently disclosed antibodies may be accomplished by altering the amino acid sequence to contain glycosylation site consensus sequences; such techniques are well known in the art. Another means of increasing the number of carbohydrate moieties on the antibodies is by chemical or enzymatic coupling of glycosides to the amino acid residues of the antibody. These methods are described in, e.g., WO 87/05330, and Aplin and Wriston (1981) CRC Crit. Rev. Biochem., 22:259-306. Removal of any carbohydrate moieties present on the antibodies may be accomplished chemically or enzymatically as described in the art (Hakimuddin et al. (1987) Arch. Biochem. Biophys., 259:52; Edge et al. (1981) Anal. Biochem., 118:131; and Thotakura et al. (1987) Meth. Enzymol., 138:350). See, e.g., U.S. Pat. No. 5,869,046 for a modification that increases in vivo half-life by providing a salvage receptor binding epitope.

In one embodiment, an anti-x-Id antibody has one or more CDR sequences (e.g., a Chothia, an enhanced Chothia, or Kabat CDR) that differ from those described herein. In one embodiment, an anti-x-Id antibody has one or more CDR sequences include amino acid changes, such as substitutions of 1, 2, 3, or 4 amino acids if a CDR is 5-7 amino acids in length, or substitutions of 1, 2, 3, 4, or 5, of amino acids in the sequence of a CDR if a CDR is 8 amino acids or greater in length. The amino acid that is substituted can have similar charge, hydrophobicity, or stereochemical characteristics. In some embodiments, the amino acid substitution(s) is a conservative substitution. A “conservative amino acid substitution” is one in which the amino acid residue is replaced with an amino acid residue having a side chain with a similar charge. Families of amino acid residues having side chains with similar charges have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine), and aromatic side chains (e.g., tyrosine, phenylalanine, tryptophan, histidine). In other embodiments, the amino acid substitution(s) is a non-conservative substitution. The antibody or antibody fragments thereof that contain the substituted CDRs can be screened to identify antibodies of interest.

Unlike in CDRs, more substantial changes in structure framework regions (FRs) can be made without adversely affecting the binding properties of an antibody. Changes to FRs include, but are not limited to, humanizing a nonhuman-derived framework or engineering certain framework residues that are important for antigen contact or for stabilizing the binding site, e.g., changing the class or subclass of the constant region, changing specific amino acid residues which might alter an effector function such as Fc receptor binding (Lund et al., J Immun., 147:26S7-62 (1991); Morgan et al., Immunology, 86:319-24 (199S)), or changing the species from which the constant region is derived.

Another type of antibody variant is an amino acid substitution variant. These variants have at least one amino acid residue in the antibody molecule replaced by a different residue. For example, the sites of greatest interest for substitutional mutagenesis of antibodies include the hypervariable regions, but framework region (FR) alterations are also contemplated.

A useful method for the identification of certain residues or regions of the anti-x-Id antibody that are preferred locations for substitution, i.e., mutagenesis, is alanine scanning mutagenesis. See Cunningham & Wells, 244 SCIENCE 1081-85 (1989). Briefly, a residue or group of target residues are identified (e.g., charged residues such as arg, asp, his, lys, and glu) and replaced by a neutral or negatively charged amino acid (most preferably alanine or polyalanine) to affect the interaction of the amino acids with antigen. The amino acid locations demonstrating functional sensitivity to the substitutions are refined by introducing further or other variants at, or for, the sites of substitution. Thus, while the site for introducing an amino acid sequence variation is predetermined, the nature of the mutation per se need not be predetermined. For example, to analyze the performance of a mutation at a given site, alanine scanning or random mutagenesis may be conducted at the target codon or region and the expressed antibody variants screened for the desired activity.

Substantial modifications in the biological properties of the antibody can be accomplished by selecting substitutions that differ significantly in their effect on, maintaining (i) the structure of the polypeptide backbone in the area of the substitution, for example, as a sheet or helical conformation, (ii) the charge or hydrophobicity of the molecule at the target site, or (iii) the bulk of the side chain. Naturally occurring residues are divided into groups based on common side-chain properties:

(1) hydrophobic: norleucine, met, ala, val, leu, ile;

(2) neutral hydrophilic: cys, ser, thr;

(3) acidic: asp, glu;

(4) basic: asn, gln, his, lys, arg;

(5) residues that influence chain orientation: gly, pro; and

(6) aromatic: trp, tyr, phe.

Non-conservative substitutions will entail exchanging a member of one of these classes for another class. Conservative substitutions involve exchanging of amino acids within the same class.

Any cysteine residue not involved in maintaining the proper conformation of the antibody also may be substituted, generally with serine, to improve the oxidative stability of the molecule and prevent aberrant crosslinking. Conversely, cysteine bond(s) may be added to the antibody to improve its stability, particularly where the antibody is an immunoglobulin fragment such as an Fv fragment.

Another type of substitutional variant involves substituting one or more hypervariable region residues of a parent antibody. Generally, the resulting variant(s), i.e., functional equivalents as defined above, selected for further development will have improved biological properties relative to the parent antibody from which they are generated. A convenient way for generating such substitutional variants is by affinity maturation using phage display. Briefly, several hypervariable region sites (e.g., 6-7 sites) are mutated to generate all possible amino substitutions at each site. The antibody variants thus generated are displayed in a monovalent fashion from filamentous phage particles as fusions to the gene III product of M13 packaged within each particle. The phage-displayed variants are then screened for their biological activity (e.g., binding affinity) as herein disclosed.

In order to identify candidate hypervariable region sites for modification, alanine-scanning mutagenesis may be performed to identify hypervariable region residues contributing significantly to antigen binding. Alternatively, or additionally, it may be beneficial to analyze a crystal structure of the antibody-antigen complex to identify contact points between the antibody and antigen. Such contact residues and neighboring residues are candidates for substitution according to the techniques elaborated herein. Once generated, the panel of variants is subjected to screening as described herein and antibodies with superior properties in one or more relevant assays may be selected for further development.

It may be desirable to modify the antibodies of the present invention, i.e., create functional equivalents, with respect to effector function, e.g., so as to enhance antigen-dependent cell-mediated cyotoxicity (ADCC) and/or complement dependent cytotoxicity (CDC) of the antibody. This may be achieved by introducing one or more amino acid substitutions in an Fc region of an antibody. Alternatively or additionally, cysteine residue(s) may be introduced in the Fc region, thereby allowing interchain disulfide bond formation in this region. The homodimeric antibody thus generated may have improved internalization capability and/or increased complement-mediated cell killing and antibody-dependent cellular cytotoxicity (ADCC). Caron et al., 176 J. EXP MED. 1191-95 (1992); Shopes, 148 J. IMMUNOL. 2918-22 (1992). Homodimeric antibodies with enhanced anti-tumor activity may also be prepared using heterobifunctional cross-linkers as described in Wolff et al., 53 CANCER RESEARCH 2560-65 (1993). Alternatively, an antibody can be engineered which has dual Fc regions and may thereby have enhanced complement lysis and ADCC capabilities. Stevenson et al., 3 ANTI-CANCER D RUG D ESIGN 219-30 (1989).

To increase the serum half-life of an antibody, one may incorporate a salvage receptor binding epitope into the antibody (especially an immunoglobulin fragment) as described in, for example, U.S. Pat. No. 5,739,277. As used herein, the term “salvage receptor binding epitope” refers to an epitope of the Fc region of an IgG molecule (e.g., IgG1, IgG2, IgG3, or IgG4) that is responsible for increasing the in vivo serum half-life of the IgG molecule.

E. Methods of Producing Anti-x-Id Antibodies

The anti-x-Id antibodies (or antigen binding domain(s) of an antibody or functional fragment thereof) of this disclosure may be produced in bacterial or eukaryotic cells. To produce the polypeptide of interest, a polynucleotide encoding the polypeptide is constructed, introduced into an expression vector, and then expressed in suitable host cells. Standard molecular biology techniques are used to prepare the recombinant expression vector, transfect the host cells, select for transformants, culture the host cells and recover the antibody.

If the antibody is to be expressed in bacterial cells (e.g., E. coli), the expression vector should have characteristics that permit amplification of the vector in the bacterial cells. Additionally, when E. coli such as JM109, DH5a, HB101, or XL I-Blue is used as a host, the vector must have a promoter, for example, a lacZ promoter (Ward et al., 341:544-546 (1989), araB promoter (Better et al., Science, 240: 1041-1043 (1988)), or T7 promoter that can allow efficient expression in E. coli. Examples of such vectors include, for example, M13-series vectors, pUC-series vectors, pBR322, pBluescript, pCR-Script, pGEX-5X-1 (Pharmacia), “QIAexpress system” (QIAGEN), pEGFP, and pET (when this expression vector is used, the host is preferably BL21 expressing T7 RNA polymerase). The expression vector may contain a signal sequence for antibody secretion. For production into the periplasm of E. coli, the pelB signal sequence (Lei et al., J. Bacteriol., 169:4379 (1987)) may be used as the signal sequence for antibody secretion. For bacterial expression, calcium chloride methods or electroporation methods may be used to introduce the expression vector into the bacterial cell.

If the antibody is to be expressed in animal cells such as CHO, COS, 293, 293T, and NIH3T3 cells, the expression vector includes a promoter necessary for expression in these cells, for example, an SV40 promoter (Mulligan et al., Nature, 277:108 (1979)), MMLV-LTR promoter, EFla promoter (Mizushima et al., Nucleic Acids Res., 18:5322 (1990)), or CMV promoter. In addition to the nucleic acid sequence encoding the immunoglobulin or domain thereof, the recombinant expression vectors may carry additional sequences, such as sequences that regulate replication of the vector in host cells (e.g., origins of replication) and selectable marker genes. The selectable marker gene facilitates selection of host cells into which the vector has been introduced (see e.g., U.S. Pat. Nos. 4,399,216, 4,634,665 and 5,179,017). For example, typically the selectable marker gene confers resistance to drugs, such as G418, hygromycin, or methotrexate, on a host cell into which the vector has been introduced. Examples of vectors with selectable markers include pMAM, pDR2, pBK-RSV, pBK-CMV, pOPRSV, and pOP13.

In one embodiment, the antibodies are produced in mammalian cells. Exemplary mammalian host cells for expressing a polypeptide include Chinese Hamster Ovary (CHO cells) (including dhfr− CHO cells, described in Urlaub and Chasin (1980) Proc. Natl. Acad. Sci. USA 77:4216-4220, used with a DHFR selectable marker, e.g., as described in Kaufman and Sharp (1982) Mol. Biol. 159:601 621), human embryonic kidney 293 cells (e.g., 293, 293E, 293T), COS cells, NIH3T3 cells, lymphocytic cell lines, e.g., NSO myeloma cells and SP2 cells, and a cell from a transgenic animal, e.g., a transgenic mammal. For example, the cell is a mammary epithelial cell.

The antibodies of the present disclosure can be isolated from inside or outside (such as medium) of the host cell and purified as substantially pure and homogenous antibodies. Methods for isolation and purification commonly used for polypeptides may be used for the isolation and purification of antibodies described herein, and are not limited to any particular method. Antibodies may be isolated and purified by appropriately selecting and combining, for example, column chromatography, filtration, ultrafiltration, salting out, solvent precipitation, solvent extraction, distillation, immunoprecipitation, SDS-polyacrylamide gel electrophoresis, isoelectric focusing, dialysis, and recrystallization. Chromatography includes, for example, affinity chromatography, ion exchange chromatography, hydrophobic chromatography, gel filtration, reverse-phase chromatography, and adsorption chromatography (Strategies for Protein Purification and Characterization: A Laboratory Course Manual. Ed Daniel R. Marshak et al., Cold Spring Harbor Laboratory Press, 1996). Chromatography can be carried out using liquid phase chromatography such as HPLC and FPLC. Columns used for affinity chromatography include protein A column and protein G column. Examples of columns using protein A column include Hyper D, POROS, and Sepharose FF (GE Healthcare Biosciences). The present disclosure also includes antibodies that are highly purified using these purification methods.

The present disclosure also provides a nucleic acid molecule or a set of nucleic acid molecules encoding an anti-x-Id antibody or antigen binding molecule thereof disclosed herein. In one embodiment, the invention includes a nucleic acid molecule encoding a polypeptide chain, which comprises a light chain of an anti-x-Id antibody or antigen-binding molecule thereof as described herein. In one embodiment, the invention includes a nucleic acid molecule encoding a polypeptide chain, which comprises a heavy chain of an anti-x-Id antibody or antigen-binding molecule thereof as described herein.

Also provided are a vector or a set of vectors comprising such nucleic acid molecule or the set of the nucleic acid molecules or a complement thereof, as well as a host cell comprising the vector.

The instant disclosure also provides a method for producing an x-Id or antigen-binding molecule thereof or chimeric molecule disclosed herein, such method comprising culturing the host cell disclosed herein and recovering the antibody, antigen-binding molecule thereof, or the chimeric molecule from the culture medium.

A variety of methods are available for recombinantly producing an x-Id antibody or antigen-binding molecule thereof disclosed herein, or a chimeric molecule disclosed herein. It will be understood that because of the degeneracy of the code, a variety of nucleic acid sequences will encode the amino acid sequence of the polypeptide. The desired polynucleotide can be produced by de novo solid-phase DNA synthesis or by PCR mutagenesis of an earlier prepared polynucleotide.

For recombinant production, a polynucleotide sequence encoding a polypeptide (e.g., an x-Id antibody or antigen-binding molecule thereof disclosed herein, or any of the chimeric molecules disclosed herein) is inserted into an appropriate expression vehicle, i.e., a vector which contains the necessary elements for the transcription and translation of the inserted coding sequence, or in the case of an RNA viral vector, the necessary elements for replication and translation.

The nucleic acid encoding the polypeptide (e.g., an x-Id antibody or antigen-binding molecule thereof disclosed herein, or any of the chimeric molecules disclosed herein) is inserted into the vector in proper reading frame. The expression vector is then transfected into a suitable target cell which will express the polypeptide. Transfection techniques known in the art include, but are not limited to, calcium phosphate precipitation (Wigler et al. 1978, Cell 14:725) and electroporation (Neumann et al. 1982, EMBO J. 1:841). A variety of host-expression vector systems can be utilized to express the polypeptides described herein (e.g., an x-Id antibody or antigen-binding molecule thereof disclosed herein, or any of the chimeric molecules disclosed herein) in eukaryotic cells. In one embodiment, the eukaryotic cell is an animal cell, including mammalian cells (e.g., 293 cells, PerC6, CHO, BHK, Cos, HeLa cells). When the polypeptide is expressed in a eukaryotic cell, the DNA encoding the polypeptide (e.g., an x-Id antibody or antigen-binding molecule thereof disclosed herein, or any of the chimeric molecules disclosed herein) can also code for a signal sequence that will permit the polypeptide to be secreted. One skilled in the art will understand that while the polypeptide is translated, the signal sequence is cleaved by the cell to form the mature chimeric molecule. Various signal sequences are known in the art and familiar to the skilled practitioner. Alternatively, where a signal sequence is not included, the polypeptide (e.g., an x-Id antibody or antigen-binding molecule thereof disclosed herein, or any of the chimeric molecules disclosed herein) can be recovered by lysing the cells.

F. CDR3 Substitutes

The present invention contemplates CDR3 amino acid substitutes of the x-clonotype: CARQEDTAMVYYFDYW (SEQ ID NO:1) for treating type 1 diabetes and other autoimmune diseases, as well as identifying individuals at-risk for developing T1D. In certain embodiments, CDR3 substitutes can include substituted derivatives that positively modulate the antigenic activity of the x-clonotype by increasing binding or interactions of the x-clonotype or antibodies bearing the x-clonotype or related sequences in the hypervariable region (i.e., CDR3) to MHC class II molecules or to TCR. These derivatives can be used as modulators or vaccine modalities. These substitutions include, for example, alanine scan derivatives at the different sequence positions of the x-clonotype, as well as non-conservative substitutions at the different sequence positions of the x-clonotype. In other embodiments, CDR3 substitutes include substituted derivatives that negatively modulate the antigenic activity of the x-clonotype by decreasing or abrogating binding or interactions of the x-clonotype or antibodies bearing the x-clonotype in the hypervariable region (CDR3) with MHC class II molecules or to TCR. These substitutions include, for example, alanine scan derivatives at the different sequence positions of the x-clonotype, as well as non-conservative substitutions at the different sequence positions of the x-clonotype. The present invention also contemplates amino acid substitutions of the antibodies described herein.

G. Pharmaceutical Compositions

The present disclosure also provides pharmaceutical compositions comprising one or more of: (i) an x-Id antibody or antigen-binding molecule thereof disclosed herein; (ii) a nucleic acid molecule or the set of nucleic acid molecules encoding an x-Id antibody or antigen-binding molecule as disclosed herein; or (iii) a vector or set of vectors disclosed herein, and a pharmaceutically acceptable carrier.

Anti-x-Id antibodies or fragments thereof described herein can be formulated as a pharmaceutical composition for administration to a subject, e.g., to treat a disorder described herein. Typically, a pharmaceutical composition includes a pharmaceutically acceptable carrier. As used herein, “pharmaceutically acceptable carrier” includes any and all solvents, dispersion media, coatings, antibacterial and antifungal agents, isotonic and absorption delaying agents, and the like that are physiologically compatible. The composition can include a pharmaceutically acceptable salt, e.g., an acid addition salt or a base addition salt (see e.g., Berge, S. M., et al. (1977) J. Pharm. Sci. 66:1-19).

Pharmaceutical formulation is a well-established art, and is further described, e.g., in Gennaro (ed.), Remington: The Science and Practice of Pharmacy, 20th ed., Lippincott, Williams & Wilkins (2000) (ISBN: 0683306472); Ansel et al., Pharmaceutical Dosage Forms and Drug Delivery Systems, 7th Ed., Lippincott Williams & Wilkins Publishers (1999) (ISBN: 0683305727); and Kibbe (ed.), Handbook of Pharmaceutical Excipients American Pharmaceutical Association, 3rd ed. (2000) (ISBN: 091733096X).

The pharmaceutical compositions may be in a variety of forms. These include, for example, liquid, semi-solid and solid dosage forms, such as liquid solutions (e.g., injectable and infusible solutions), dispersions or suspensions, tablets, pills, powders, liposomes and suppositories. The preferred form can depend on the intended mode of administration and therapeutic application. Typically compositions for the agents described herein are in the form of injectable or infusible solutions.

In one embodiment, an antibody described herein is formulated with excipient materials, such as sodium citrate, sodium dibasic phosphate heptahydrate, sodium monobasic phosphate, Tween®-80, and a stabilizer. It can be provided, for example, in a buffered solution at a suitable concentration and can be stored at 2-8° C. In some other embodiments, the pH of the composition is between about 5.5 and 7.5 (e.g., 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5).

The pharmaceutical compositions can also include agents that reduce aggregation of the antibody when formulated. Examples of aggregation reducing agents include one or more amino acids selected from the group consisting of methionine, arginine, lysine, aspartic acid, glycine, and glutamic acid. These amino acids may be added to the formulation to a concentration of about 0.5 mM to about 145 mM (e.g., 0.5 mM, 1 mM, 2 mM, 5 mM, 10 mM, 25 mM, 50 mM, 100 mM). The pharmaceutical compositions can also include a sugar (e.g., sucrose, trehalose, mannitol, sorbitol, or xylitol) and/or atonicity modifier (e.g., sodium chloride, mannitol, or sorbitol) and/or a surfactant (e.g., polysorbate-20 or polysorbate-80).

The composition can be formulated as a solution, microemulsion, dispersion, liposome, or other ordered structure suitable for stable storage at high concentration. Sterile injectable solutions can be prepared by incorporating an agent described herein in the required amount in an appropriate solvent with one or a combination of ingredients enumerated above, as required, followed by filtered sterilization.

Generally, dispersions are prepared by incorporating an agent described herein into a sterile vehicle that contains a basic dispersion medium and the required other ingredients from those enumerated above. In the case of sterile powders for the preparation of sterile injectable solutions, the preferred methods of preparation are vacuum drying and freeze drying that yield a powder of an agent described herein plus any additional desired ingredient from a previously sterile-filtered solution thereof. The proper fluidity of a solution can be maintained, for example, by the use of a coating such as lecithin, by the maintenance of the required particle size in the case of dispersion and by the use of surfactants. Prolonged absorption of injectable compositions can be brought about by including in the composition an agent that delays absorption, for example, monostearate salts and gelatin.

In certain embodiments, the antibodies may be prepared with a carrier that will protect the compound against rapid release, such as a controlled release formulation, including implants, and microencapsulated delivery systems. Biodegradable, biocompatible polymers can be used, such as ethylene vinyl acetate, polyanhydrides, polyglycolic acid, collagen, polyorthoesters, and polylactic acid. Many methods for the preparation of such formulations are patented or generally known. See, e.g., Sustained and Controlled Release Drug Delivery Systems, J. R. Robinson, ed., Marcel Dekker, Inc., New York (1978).

In one embodiment, the pharmaceutical formulation comprises an antibody at a concentration of about 0.005 mg/mL to 500 mg/mL (e.g., 0.005 mg/ml, 0.01 mg/ml, 0.05 mg/ml, 0.1 mg/ml, 0.5 mg/mL, 1 mg/mL, 5 mg/mL, 10 mg/mL, 25 mg/mL, 30 mg/mL, 35 mg/mL, 40 mg/mL, 45 mg/mL, 50 mg/mL, 55 mg/mL, 60 mg/mL, 65 mg/mL, 70 mg/mL, 75 mg/mL, 80 mg/mL, 85 mg/mL, 90 mg/mL, 95 mg/mL, 100 mg/mL, 125 mg/mL, 150 mg/mL, 175 mg/mL, 200 mg/mL, 250 mg/mL, 300 mg/mL, 350 mg/mL, 400 mg/mL, 450 mg/mL, 500 mg/mL), formulated with a pharmaceutically acceptable carrier. In some embodiments, the antibody is formulated in sterile distilled water or phosphate buffered saline. The pH of the pharmaceutical formulation may be between 5.5 and 7.5 (e.g., 5.5, 5.6, 5.7, 5.8, 5.9, 6.0, 6.1, 6.2 6.3, 6.4 6.5, 6.6 6.7, 6.8, 6.9 7.0, 7.1, 7.3, 7.4, 7.5).

A pharmaceutical composition may include a “therapeutically effective amount” of an agent described herein. Such effective amounts can be determined based on the effect of the administered agent, or the combinatorial effect of agents if more than one agent is used. A therapeutically effective amount of an agent may also vary according to factors such as the disease state, age, sex, and weight of the individual, and the ability of the compound to elicit a desired response in the individual, e.g., amelioration of at least one disorder parameter or amelioration of at least one symptom of the disorder. A therapeutically effective amount is also one in which any toxic or detrimental effects of the composition are outweighed by the therapeutically beneficial effects.

1. Administration

The antibodies or antigen-binding fragment thereof, or nucleic acids encoding same of the disclosure can be administered to a subject, e.g., a subject in need thereof, for example, a human or animal subject, by a variety of methods. For many applications, the route of administration is one of: intravenous injection or parenteral, infusion (IV), subcutaneous injection (SC), intraperitoneally (IP), or intramuscular injection, intratumor (IT). Other modes of parenteral administration can also be used. Examples of such modes include: intraarterial, intrathecal, intracapsular, intraorbital, intracardiac, intradermal, transtracheal, subcuticular, intraarticular, subcapsular, subarachnoid, intraspinal, and epidural and intrastemal injection.

In one embodiment, the route of administration of the antibodies of the invention is parenteral. The term parenteral as used herein includes intravenous, intraarterial, intraperitoneal, intramuscular, subcutaneous, rectal or vaginal administration. The intravenous form of parenteral administration is preferred. While all these forms of administration are clearly contemplated as being within the scope of the invention, a form for administration would be a solution for injection, in particular for intravenous or intraarterial injection or drip. Usually, a suitable pharmaceutical composition for injection can comprise a buffer (e.g., acetate, phosphate or citrate buffer), a surfactant (e.g., polysorbate), optionally a stabilizer agent (e.g., human albumin), etc. However, in other methods compatible with the teachings herein, the polypeptides can be delivered directly to the site of the adverse cellular population thereby increasing the exposure of the diseased tissue to the therapeutic agent.

Preparations for parenteral administration include sterile aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media.

Pharmaceutically acceptable carriers include, but are not limited to, 0.01-0.1M and preferably 0.05M phosphate buffer or 0.8% saline. Other common parenteral vehicles include sodium phosphate solutions, Ringer's dextrose, dextrose and sodium chloride, lactated Ringer's, or fixed oils. Intravenous vehicles include fluid and nutrient replenishers, electrolyte replenishers, such as those based on Ringer's dextrose, and the like. Preservatives and other additives can also be present such as for example, antimicrobials, antioxidants, chelating agents, and inert gases and the like.

More particularly, pharmaceutical compositions suitable for injectable use include sterile aqueous solutions (where water soluble) or dispersions and sterile powders for the extemporaneous preparation of sterile injectable solutions or dispersions. In such cases, the composition must be sterile and should be fluid to the extent that easy syringability exists. It should be stable under the conditions of manufacture and storage and will preferably be preserved against the contaminating action of microorganisms, such as bacteria and fungi. The carrier can be a solvent or dispersion medium containing, for example, water, ethanol, polyol (e.g., glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants.

Prevention of the action of microorganisms can be achieved by various antibacterial and antifungal agents, for example, parabens, chlorobutanol, phenol, ascorbic acid, thimerosal and the like. In many cases, it will be preferable to include isotonic agents, for example, sugars, polyalcohols, such as mannitol, sorbitol, or sodium chloride in the composition. Prolonged absorption of the injectable compositions can be brought about by including in the composition an agent which delays absorption, for example, aluminum monostearate and gelatin.

In any case, sterile injectable solutions can be prepared by incorporating an active compound (e.g., a polypeptide by itself or in combination with other active agents) in the required amount in an appropriate solvent with one or a combination of ingredients enumerated herein, as required, followed by filtered sterilization.

Generally, dispersions are prepared by incorporating the active compound into a sterile vehicle, which contains a basic dispersion medium and the required other ingredients from those enumerated above. In the case of sterile powders for the preparation of sterile injectable solutions, the preferred methods of preparation are vacuum drying and freeze-drying, which yields a powder of an active ingredient plus any additional desired ingredient from a previously sterile-filtered solution thereof. The preparations for injections are processed, filled into containers such as ampoules, bags, bottles, syringes or vials, and sealed under aseptic conditions according to methods known in the art. Further, the preparations can be packaged and sold in the form of a kit. Such articles of manufacture will preferably have labels or package inserts indicating that the associated compositions are useful for treating a subject suffering from, or predisposed to dotting disorders.

Effective doses of the compositions of the present disclosure, for the treatment of conditions vary depending upon many different factors, including means of administration, target site, physiological state of the patient, whether the patient is human or an animal, other medications administered, and whether treatment is prophylactic or therapeutic. Usually, the patient is a human but non-human mammals including transgenic mammals can also be treated. Treatment dosages can be titrated using routine methods known to those of skill in the art to optimize safety and efficacy.

The route and/or mode of administration of the anti-x-Id antibody or fragment thereof can also be tailored for the individual case, e.g., by monitoring the subject.

The antibody or fragment thereof can be administered as a fixed dose, or in a mg/kg dose. The dose can also be chosen to reduce or avoid production of antibodies against the anti-x-Id antibody or fragment thereof. Dosage regimens are adjusted to provide the desired response, e.g., a therapeutic response or a combinatorial therapeutic effect. Generally, doses of the antibody or fragment thereof (and optionally a second agent) can be used in order to provide a subject with the agent in bioavailable quantities. For example, doses in the range of 0.1-100 mg/kg, 0.5-100 mg/kg, 1 mg/kg-100 mg/kg, 0.5-20 mg/kg, 0.1-10 mg/kg, or 1-10 mg/kg can be administered. Other doses can also be used. In certain embodiments, a subject in need of treatment with an antibody or fragment thereof is administered the antibody or fragment thereof at a dose of between about 1 mg/kg to about 30 mg/kg. In some embodiments, a subject in need of treatment with anti-x-Id antibody or fragment thereof is administered the antibody or fragment thereof at a dose of 1 mg/kg, 2 mg/kg, 4 mg/kg, 5 mg/kg, 7 mg/kg 10 mg/kg, 12 mg/kg, 15 mg/kg, 20 mg/kg, 25 mg/kg, 28 mg/kg, 30 mg/kg, 35 mg/kg, 40 mg/kg, or 50 mg/kg. In a specific embodiment, the antibody or fragment thereof is administered subcutaneously at a dose of 1 mg/kg to 3 mg/kg. In another embodiment, the antibody or fragment thereof is administered intravenously at a dose of between 4 mg/kg and 30 mg/kg.

A composition may comprise about 1 mg/mL to 100 mg/ml or about 10 mg/mL to 100 mg/ml or about 50 to 250 mg/mL or about 100 to 150 mg/ml or about 100 to 250 mg/ml of the antibody or fragment thereof.

Dosage unit form or “fixed dose” as used herein refers to physically discrete units suited as unitary dosages for the subjects to be treated; each unit contains a predetermined quantity of antibody or fragment thereof calculated to produce the desired therapeutic effect in association with the required pharmaceutical carrier and optionally in association with the other agent. Single or multiple dosages may be given. Alternatively, or in addition, the antibody or fragment thereof may be administered via continuous infusion.

An antibody or fragment thereof dose can be administered, e.g., at a periodic interval over a period of time (a course of treatment) sufficient to encompass at least 2 doses, 3 doses, 5 doses, 10 doses, or more, e.g., once or twice daily, or about one to four times per week, or preferably weekly, biweekly (every two weeks), every three weeks, monthly, e.g., for between about 1 to 12 weeks, preferably between 2 to 8 weeks, more preferably between about 3 to 7 weeks, and even more preferably for about 4, 5, or 6 weeks. Factors that may influence the dosage and timing required to effectively treat a subject, include, e.g., the stage or severity of the disease or disorder, formulation, route of delivery, previous treatments, the general health and/or age of the subject, and other diseases present. Moreover, treatment of a subject with a therapeutically effective amount of a compound can include a single treatment or, preferably, can include a series of treatments.

If a subject is at risk for developing a disorder described herein, the antibody or fragment thereof can be administered before the full onset of the disorder, e.g., as a preventative measure. The duration of such preventative treatment can be a single dosage of the antibody or fragment thereof or the treatment may continue (e.g., multiple dosages). For example, a subject at risk for the disorder or who has a predisposition for the disorder may be treated with the antibody or fragment thereof for days, weeks, months, or even years so as to prevent the disorder from occurring or fulminating.

In certain embodiments, the antibody or fragment thereof is administered subcutaneously at a concentration of about 1 mg/mL to about 500 mg/mL (e.g., 1 mg/mL, 2 mg/mL, 3 mg/mL, 4 mg/mL, 5 mg/mL, 10 mg/mL, 15 mg/mL, 20 mg/mL, 25 mg/mL, 30 mg/mL, 35 mg/mL, 40 mg/mL, 45 mg/mL, 50 mg/mL, 55 mg/mL, 60 mg/mL, 65 mg/mL, 70 mg/mL, 75 mg/mL, 80 mg/mL, 85 mg/mL, 90 mg/mL, 95 mg/mL, 100 mg/mL, 125 mg/mL, 150 mg/mL, 175 mg/mL, 200 mg/mL, 225 mg/mL, 250 mg/mL, 275 mg/mL, 300 mg/mL, 325 mg/mL, 350 mg/mL, 400 mg/mL, 450 mg/mL). In one embodiment, the anti-x-Id antibody or fragment thereof is administered subcutaneously at a concentration of 50 mg/mL. In another embodiment, the antibody or fragment thereof is administered intravenously at a concentration of about 1 mg/mL to about 500 mg/mL. In one embodiment, the antibody or fragment thereof is administered intravenously at a concentration of 50 mg/mL.

Doses intermediate in the above ranges are also intended to be within the scope of the invention. Subjects can be administered such doses daily, on alternative days, weekly or according to any other schedule determined by empirical analysis. An exemplary treatment entails administration in multiple dosages over a prolonged period, for example, of at least six months. In some methods, two or more polypeptides can be administered simultaneously, in which case the dosage of each polypeptide administered falls within the ranges indicated.

Polypeptides of the invention can be administered on multiple occasions. Intervals between single dosages can be daily, weekly, monthly or yearly. Intervals can also be irregular as indicated by measuring blood levels of modified polypeptide or antigen in the patient. Alternatively, polypeptides can be administered as a sustained release formulation, in which case less frequent administration is required. Dosage and frequency vary depending on the half-life of the polypeptide in the patient.

The dosage and frequency of administration can vary depending on whether the treatment is prophylactic or therapeutic. In prophylactic applications, compositions containing the polypeptides of the invention or a cocktail thereof are administered to a patient not already in the disease state to enhance the patient's resistance or minimize effects of disease. Such an amount is defined to be a “prophylactic effective dose.” A relatively low dosage is administered at relatively infrequent intervals over a long period of time. Some patients continue to receive treatment for the rest of their lives.

H. Devices and Kits for Therapy

An anti-x-Id antibody or fragment thereof can be provided in a kit. In one embodiment, the kit includes (a) a container that contains a composition that includes an anti-x-Id antibody or fragment thereof as described herein, and optionally (b) informational material. The informational material can be descriptive, instructional, marketing or other material that relates to the methods described herein and/or the use of the agents for therapeutic benefit.

In an embodiment, the kit also includes a second agent for treating a disorder described herein. For example, the kit includes a first container that contains a composition that includes the anti-x-Id antibody or fragment thereof, and a second container that includes the second agent.

The informational material of the kits is not limited in its form. In one embodiment, the informational material can include information about production of the compound, molecular weight of the compound, concentration, date of expiration, batch or production site information, and so forth. In one embodiment, the informational material relates to methods of administering the anti-x-Id antibody or fragment thereof, e.g., in a suitable dose, dosage form, or mode of administration (e.g., a dose, dosage form, or mode of administration described herein), to treat a subject who has had or who is at risk for a disease as described herein. The information can be provided in a variety of formats, include printed text, computer readable material, video recording, or audio recording, or information that provides a link or address to substantive material, e.g., on the internet.

In addition to the anti-x-Id antibody or fragment thereof, the composition in the kit can include other ingredients, such as a solvent or buffer, a stabilizer, or a preservative. The anti-x-Id antibody or fragment thereof can be provided in any form, e.g., liquid, dried or lyophilized form, preferably substantially pure and/or sterile. When the agents are provided in a liquid solution, the liquid solution preferably is an aqueous solution. In certain embodiments, the anti-x-Id antibody or fragment thereof in the liquid solution is at a concentration of about 25 mg/mL to about 250 mg/mL (e.g., 40 mg/mL, 50 mg/mL, 60 mg/mL, 75 mg/mL, 85 mg/mL, 100 mg/mL, 125 mg/mL, 150 mg/mL, and 200 mg/mL). When the anti-x-Id antibody or fragment thereof is provided as a lyophilized product, the anti-x-Id antibody or fragment thereof is at about 75 mg/vial to about 200 mg/vial (e.g., 100 mg/vial, 108.5 mg/vial, 125 mg/vial, 150 mg/vial). The lyophilized powder is generally reconstituted by the addition of a suitable solvent. The solvent, e.g., sterile water or buffer (e.g., PBS), can optionally be provided in the kit.

The kit can include one or more containers for the composition or compositions containing the agents. In some embodiments, the kit contains separate containers, dividers or compartments for the composition and informational material. For example, the composition can be contained in a bottle, vial, or syringe, and the informational material can be contained in a plastic sleeve or packet. In other embodiments, the separate elements of the kit are contained within a single, undivided container. For example, the composition is contained in a bottle, vial or syringe that has attached thereto the informational material in the form of a label. In some embodiments, the kit includes a plurality (e.g., a pack) of individual containers, each containing one or more unit dosage forms (e.g., a dosage form described herein) of the agents. The containers can include a combination unit dosage, e.g., a unit that includes both the anti-x-Id antibody or fragment thereof and the second agent, e.g., in a desired ratio. For example, the kit includes a plurality of syringes, ampules, foil packets, blister packs, or medical devices, e.g., each containing a single combination unit dose. The containers of the kits can be air tight, waterproof (e.g., impermeable to changes in moisture or evaporation), and/or light-tight.

The kit optionally includes a device suitable for administration of the composition, e.g., a syringe or other suitable delivery device. The device can be provided pre-loaded with one or both of the agents or can be empty, but suitable for loading.

Without further elaboration, it is believed that one skilled in the art, using the preceding description, can utilize the present invention to the fullest extent. The following examples are illustrative only, and not limiting of the remainder of the disclosure in any way whatsoever.

### EXAMPLES

The following examples are put forth so as to provide those of ordinary skill in the art with a complete disclosure and description of how the compounds, compositions, articles, devices, and/or methods described and claimed herein are made and evaluated, and are intended to be purely illustrative and are not intended to limit the scope of what the inventors regard as their invention. Efforts have been made to ensure accuracy with respect to numbers (e.g., amounts, temperature, etc.) but some errors and deviations should be accounted for herein. Unless indicated otherwise, parts are parts by weight, temperature is in degrees Celsius or is at ambient temperature, and pressure is at or near atmospheric. There are numerous variations and combinations of reaction conditions, e.g., component concentrations, desired solvents, solvent mixtures, temperatures, pressures and other reaction ranges and conditions that can be used to optimize the product purity and yield obtained from the described process. Only reasonable and routine experimentation will be required to optimize such process conditions.

T and B cells are the two known adaptive immune cells. Here we describe a previously unknown lymphocyte that is a dual expresser (DE) of TCR and BCR and key lineage markers of B and T cells. In type 1 diabetes (T1D), DEs are predominated by one clonotype that encodes a potent CD4 T cell autoantigen in its antigen binding site (referred to as x-idiotype). Molecular dynamics simulations revealed that the x-idiotype peptide (x-Id) has an optimal binding register for diabetogenic HLA-DQ8. In concordance, synthesized x-Id peptide forms stable DQ8 complexes and potently stimulates autoreactive CD4 T cells from T1D, but not healthy controls. Moreover, x-clonotype-bearing mAbs are autoreactive against CD4 T cells and inhibit insulintetramer binding to CD4 T cells. Thus, compartmentalization of adaptive immune cells into T and B cells is not absolute and violators of this paradigm are likely key drivers of autoimmune diseases.

### Example 1: A Public BCR Present in a Unique Dual-Receptor-Expressing Lymphocyte from Type 1 Diabetes Patients Encodes a Potent T Cell Autoantigen

### Materials and Methods

Human subjects. Peripheral blood samples were obtained from donors using protocols approved by the Johns Hopkins Institutional Review Board. All donors provided written informed consent. All T1D subjects met the American Diabetes Association criteria for classification and were recruited at Johns Hopkins Comprehensive Diabetes Center. Donors with no T1D are classified as healthy controls (HCs) and were recruited from normal volunteers. The clinical characteristics of donors are summarized in a Table 1A (data not shown). The study was conducted in accordance with the declaration of Helsinki principles. Peripheral blood mononuclear cells (PBMCs) were freshly isolated using Ficoll-paque density centrifugation (GE Healthcare) gradient. Islet autoantibodies profiles and HLA genotypes of subjects (Table 1B and 1C, not shown) whose repertoires were analyzed by high-throughput were performed at the Barbara Davis Center Autoantibody/HLA Core Laboratory in Denver using established methods.

Flow cytometric analysis. Cell phenotypes were analyzed using a LSRII multicolor flow cytometer (BD Biosciences). Briefly, single cell suspensions were surface-stained for 20 min on ice with predetermined optimal concentrations of indicated fluorochrome-conjugated antibodies (Key resources table (not shown)) using established methods (Dai et al., 2015; Martina et al., 2015). Acquired samples (5×105 to 1×106 live events) were properly compensated using single color stains. Data analysis, gating, and graphical presentation were done using FlowJo software (TreeStar). Doublets were excluded from analysis using FSC-Height versus FSC-Width and SSC-Height versus SSC-Width plots. Multiple specificity controls were used. These included human FcR blocking reagent (Miltenyi Biotec), Fluorescence-Minus One (FMO) for CD5, CD19, TCR, IgD, dump gating, and isotype controls. In addition, when applicable, irrelevant cell types were used as internal biological controls and in the case of in vitro stimulation, we used unstimulated cultures as negative controls.

Imaging flow cytometry (AMNIS). Freshly isolated PBMCs were stained with FITC-conjugated anti-TCRαβ, PE-conjugated anti-IgD, APC conjugated anti-CD5, and BV421-conjugated anti-CD19 and analyzed at X60 magnification on an Image Stream flow cytometer (Amnis corporation) with low flow rate/high sensitivity using INSPIRE software. For each sample, 10,000 events were acquired. Single color controls were used for creation of a compensation matrix, to set the optimal laser power for each fluorochrome and to avoid saturation of the camera. The compensation matrix was applied to all files to correct for spectral cross-talk. Positive cutoff values were calculated on the basis of the bright detail similarity (BDS) background of TCRαβ and an irrelevant signal (for example, side scatter). Image analysis was performed with the IDEAS 6.2 software package using bright field images to set cell boundary and gating on internalized events. Compensated data files were analyzed using a gating strategy that involved selecting focused cells on the basis of gradient RMS and an aspect ratio that was consistent with single events and devoid of debris or multi-cellular events (doublets). T cell and B cell singlets were successfully identified using this strategy and the selection of good quality, focused singlets within the viewing window allowed refining of final gating. After the gating of T_{con} and B_{con} cells, individual IgD+ DE cells were identified based on their surface profile (CD19+CD5+TCR+IgD+) and analyzed for the indicated markers. Bright field imagery was collected with an LED-based bright field illuminator. Each plot was manually adjusted so that the machine noise generated at the beginning of acquisition was set to zero.

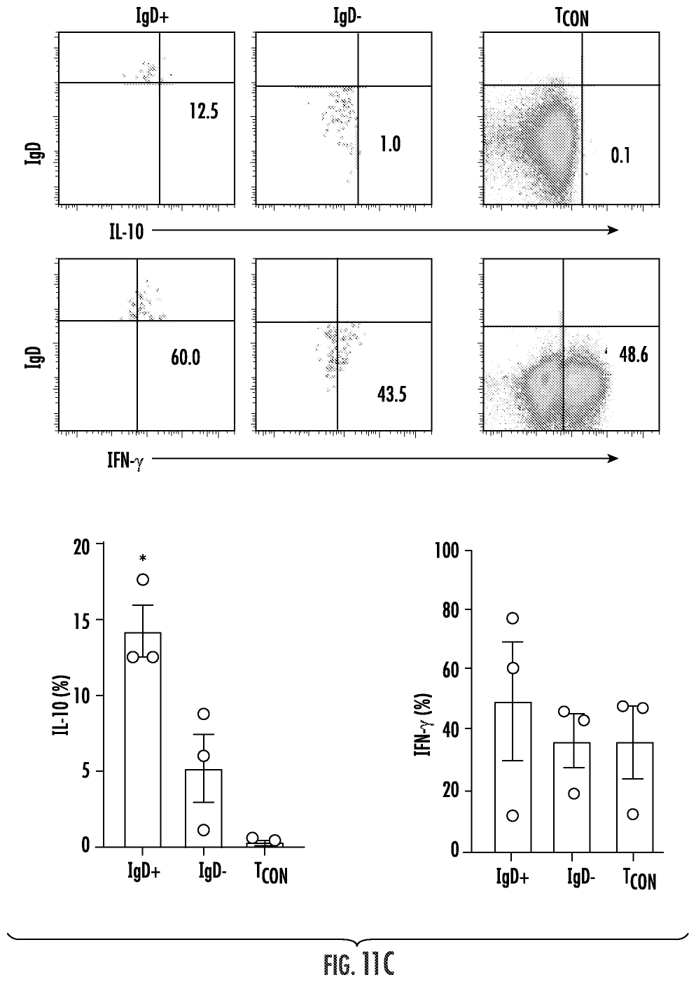

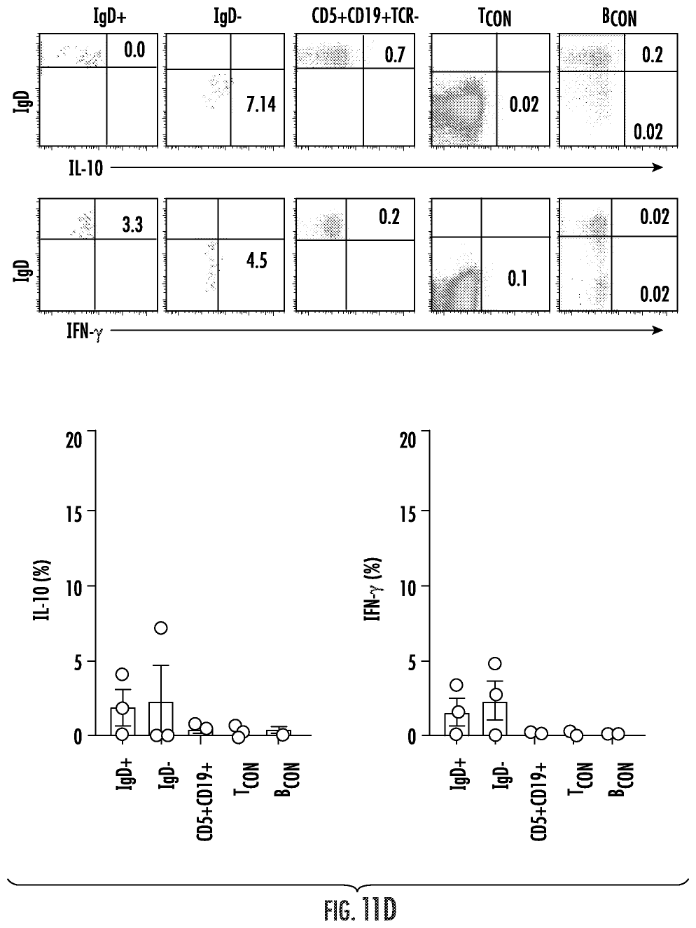

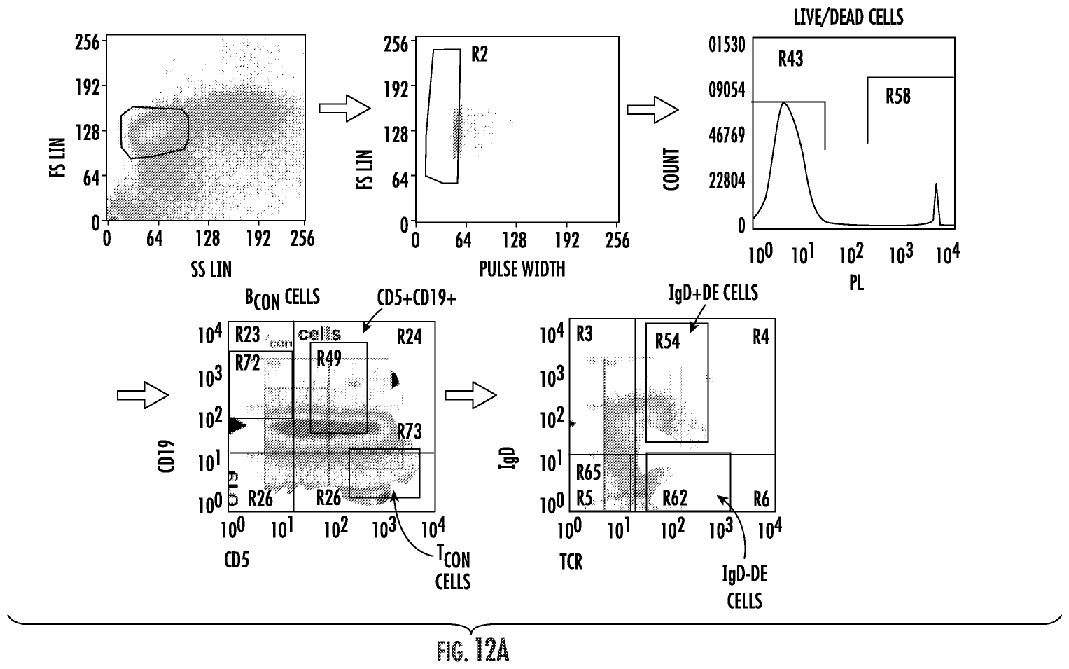

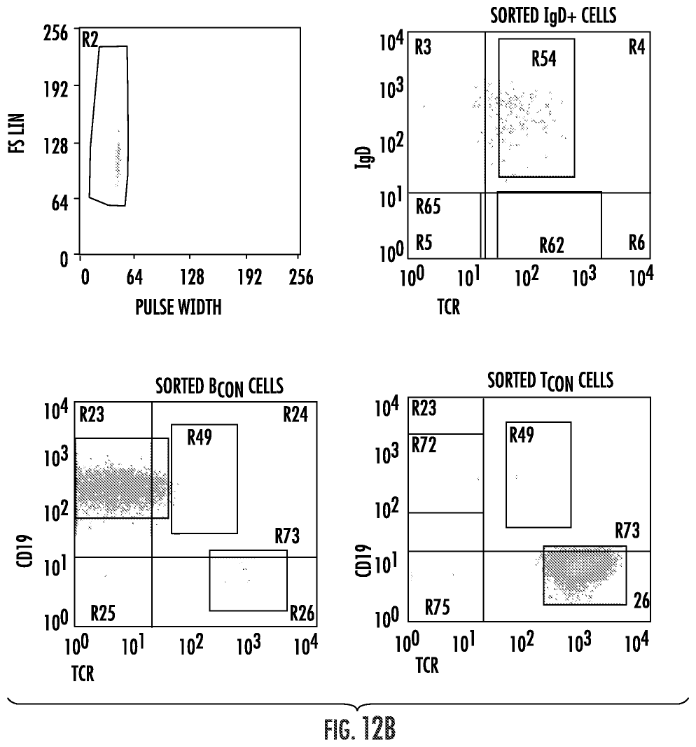

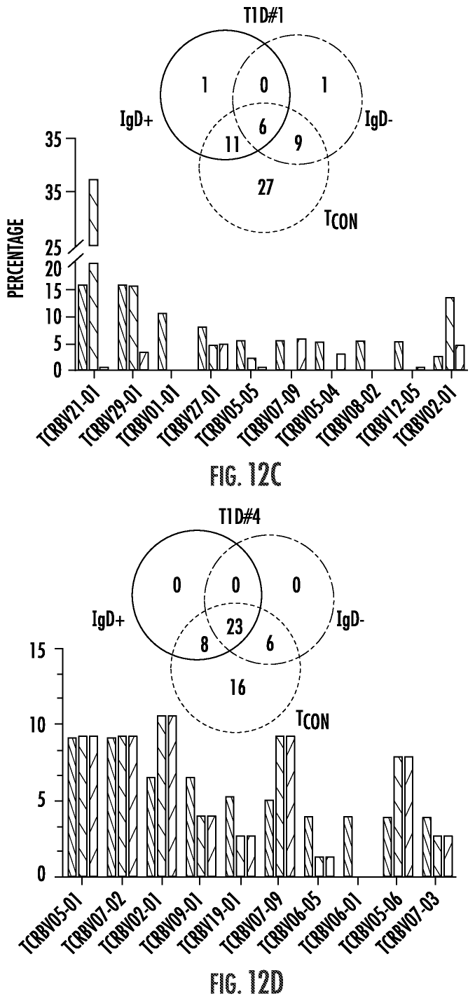

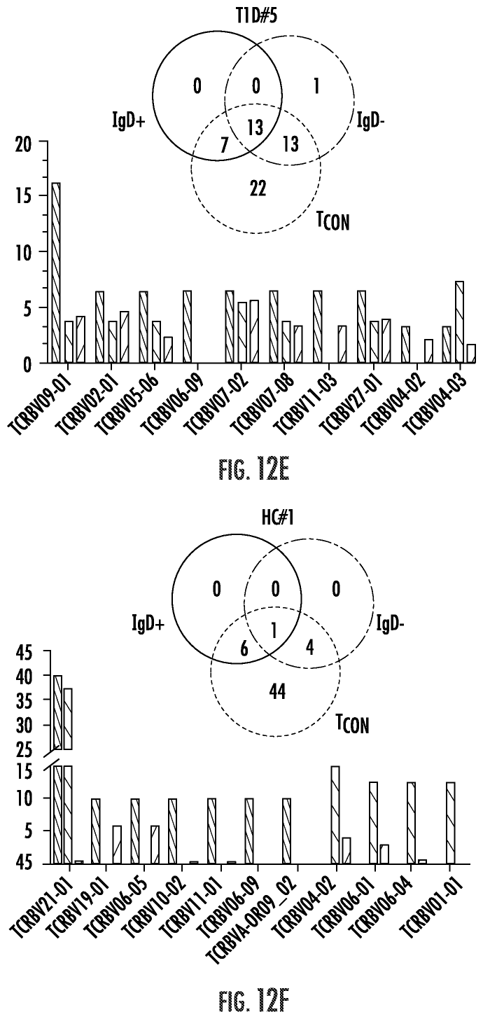

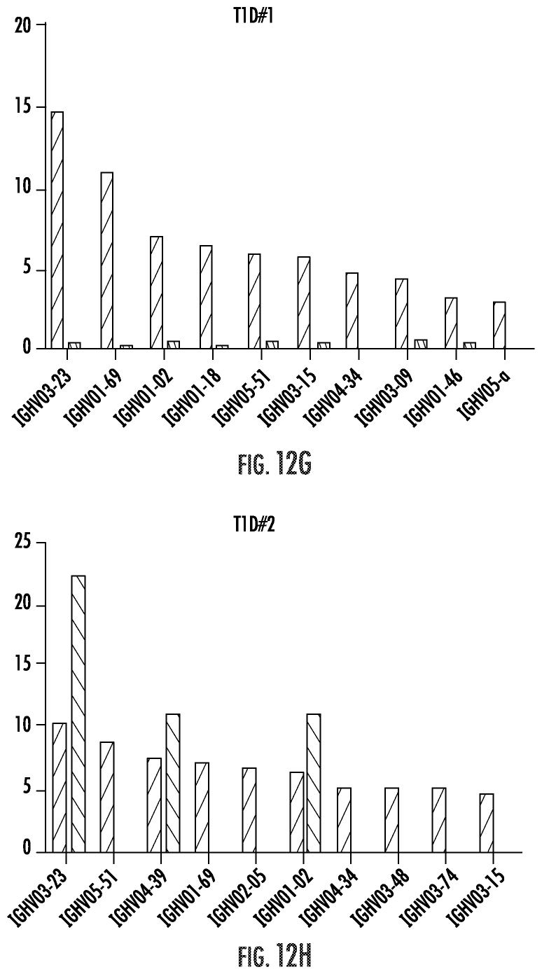

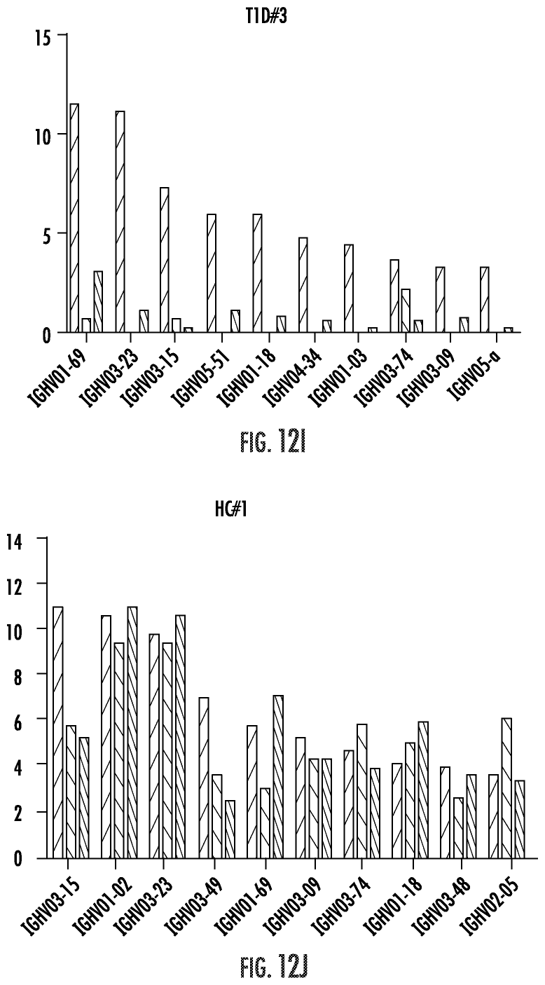

Single cell RNA-seq data generation and processing. FACS sorted single cells (see FIGS. 12A and 12B for sorting strategy) were processed with the Smart-seq2 protocol (Picelli et al., 2014) with the following modifications. RNA purification was performed prior to reverse transcription using RNAClean XP beads (Beckman Coulter). cDNA was amplified with 21 PCR cycles followed by DNA cleanup with AMPure XP beads. Libraries were prepared using the Nextera XT Library Prep kit (Illumina) using custom barcode adapters. Uniquely barcoded Libraries were sequenced together on a NextSeq 500 sequencer (Illumina).

Bioinformatic analysis of scRNA-seq Data. QC checks were performed on the scRNA-seq data with R bioconductor package scater following the methods described by Lun et al. (Lun et al., 2016). The QC metrics included library size, number of features expressed, proportions of ERCC spike-in controls, and three empty wells that were included in the experimental design as negative controls. In addition to the three empty wells, 18 out of 93 biological (B, T and DE) cells had either log-library sizes and/or log-transformed number of expressed transcripts blow the respective medians by more than 3 median absolute deviations (MADs) and were filtered out as low quality outlier samples. Another DE cell D 07 had a library size below the maximum of the three empty wells and was viewed as a low quality sample. Among the 19 low-quality biological cell samples, 12/45 are DE cells, 6/24 B_{con} cells and 1/24 T_{con} cells. All the 19 cells had library sizes lower than or comparable to the empty wells. 64 out of the 74 good quality samples have a sequencing depth of 1-3 million reads and are deemed to reach saturation while the other 10 samples have a depth between 0.7-1 million reads, good for the detection of large majority of genes (Michel et al., 2012; Wu et al., 2014; Ziegenhain et al., 2017). The sequencing assay kit also included 12 ERCC spike in controls. The 19 low quality cells had a pattern of spike-in ERCC proportions similar to the good quality ones above and did not show any increase. Assuming the majority of cells are of high quality, it suggests there is little loss of endogenous RNA in all the cells. Taken together, the analyses above suggest good overall quality of the scRNA-seq experiment.

Following a biology-guided strategy, we limited downstream analysis of the scRNA-Seq data to cells in which at least two of three housekeeping genes (PPIA, ACTB, and UBB) were detected as expressed, defined as having log 2 (RSEM value+1)>0. This resulted in 77 high quality cells. Genes preferentially expressed in either B_{con} cells, T_{con} cells, or DE cells were identified using the Template Matching method, which tests for an association between each profile and an artificial profile that represents an ideal, cluster- or condition-specific, profile using the Pearson's product moment correlation coefficient (Pavlidis and Noble, 2001). Multiple testing corrections were performed using Holm's method (Holm, 1979). To identify BCR and TCR transcripts expressed in the single cells, we used BASIC (Canzar et al., 2017). We annotated each cell according to whether BASIC was able to assemble BCR or TCR transcripts.

Polyclonal TCR stimulation. Freshly isolated PBMCs were placed onto the wells of 24-well tissue culture plates (106 cells in 1 ml complete culture medium) in the presence or absence of anti-CD3/CD28 beads (106 bead/well) and incubated at 37° C. and 5% CO2. Alternatively, plates coated with anti-CD3 (10 μg/ml) and anti-CD28 (10 μg/ml) were used (Yoneshiro et al., 2017). After 7 days in culture, viable cells were harvested, counted using trypan blue, and analyzed for the expression of indicated molecules using a BD LSRII flow cytometer. Absolute cell numbers were determined by multiplying the frequency of the indicated subset by the viable cell count.

CFSE proliferation assay. Freshly isolated PBMCs were washed twice with warm (37° C.) 1×PBS to remove serum that affect staining and the cells were resuspended in warm (37° C.) 1×PBS at a density of 1.5-2.0×106 cells/ml. Cells were labeled with 1 μM CFSE (eBioscience) for 1-2 min at 37° C. with continuous vortexing. The labeling reaction was quenched by adding chilled complete culture media. CFSE-labeled cells were washed in 1×PBS, resuspended in complete media, and plated into 24-well tissue culture plates (1.5-2.0×106 cells/well in 1 mL complete culture medium). To evaluate functionality of HLA-DQ8 molecules, we immobilized DQ8 molecules loaded with indicated peptides (x-Id, TP-Id, mimotope, native insulin and CDR3 peptide from IgD+ DE from HC #1 (referred to as h-Id) into wells of 24-well plates (10 μM) and examined their ability to stimulate CFSE-labeled CD4 T cells from among PBMCs. In parallel experiments, we activated cultures in the presence (20 uM) of mouse anti-HLA-DQ (SPV-L3; Abcam) and anti-HLA-DR (L243; Abcam) to assess MHC restriction. In similar manner, CFSE labeled cells were also stimulated in the presence or absence of the above-indicated peptides (10 μM) as soluble antigen. Alternatively, in a separate experiment, to evaluate the mAb-specific proliferative response, purified mAbR and mAbN (described later in the method) concentration of 2.5 and 5 ug, immobilized into the wells of 24-well plates, and used to stimulate CFSE-labeled PBMCs. CFSE labelled cells without stimulation and with CD3-28 stimulation were taken as specific negative and positive controls respectively. After 7 days of incubation, cultures were stained as indicated in FIG. legends and proliferation assessed by determining frequency of CFSElow CD4 T cells.

Intracellular Cytokine analysis. Single cell suspensions were stimulated for 4 h at 37° C. in 5% CO2 with phorbol 12-myristate 13-acetate (PMA) (50 ng/mL) and ionomycin (500 ng/mL) in the presence of Golgi-Plug (Saxena et al., 2017; Xiao et al., 2011). Intracellular cytokine analysis was performed using the manufacturers' instructions. Briefly, surface-stained samples were fixed, permeabilized and incubated with mAbs against indicated intracellular cytokines for 30 min on ice, washed, acquired and analyzed using the above described strategy.

Anti-IgM stimulation. Freshly isolated PBMCs (1×106) in RPMI-1640 medium were allowed to rest at 37° C. in CO2 incubator for 30 min before stimulation. BCR signaling was induced by crosslinking the BCR with 10 μg/ml goat F(ab′) 2 anti-IgM (Jackson ImmunoResearch) at 37° C. in CO2 incubator for indicated time points as described (Wang et al., 2014). Time course analysis was achieved by adding anti-IgM to each sample in reverse time points and fixing all samples in unison. To determine basal levels of phosphorylation, parallel cultures of unstimulated PBMCs were fixed at time zero. To detect phosphorylated CD79a (pIgα), cells were fixed (1.5% paraformaldehyde, 5 mins, room temperature), permeabilized (90% methanol, 10 min, 4° C.), and stained with rabbit antibodies specific for pCD79A (Igα, Tyr82) followed by PE-conjugated goat anti-rabbit IgG (Jackson ImmunoResearch Laboratories).

Cell sorting and DNA extraction for high-throughput sequencing of IGHV and TRB. For repertoire analysis, IgD+ and IgD− DE cells, B_{con}, and T_{con} cells were sorted from freshly isolated PBMCs using a FACSAria II (BD Biosciences, Bedford, Mass.). The sorting strategy and purity of isolated cell populations are shown in (FIGS. 12A and 12B). Two sorts were performed from each donor and (except HC #2) were performed at different time points with one sort used for IGHV and the second for TRB analysis. Donor characteristics, including islet autoantibodies and HLA genotypes, are shown in (Table 1, not shown). Autologous B_{con} cells were used as controls for IGVH analysis and T_{con} cells for TRB analysis. Briefly, freshly isolated PBMCs were stained for CD19, CD5, IgD, and TCRαβ for 30 min on ice, washed thoroughly, and suspended in a pre-sort buffer (BD Biosciences). Propidium iodide (PI) was added immediately prior to sorting to exclude non-viable cells. Sorted cells were collected in 50% FBS on ice. IgD+ DE cells were identified as CD19+CD5+IgD+ TCRβ+ (800-1000 cells per sort) and IgD− DE cells as CD19+CD5+IgD−TCRβ+ cells (100-200 cells per sort). B_{con} cells were identified as CD19+CD5−TCRβ− and T_{con} cells as CD19−CD5+TCRP+ cells. Total DNA was directly extracted from sorted cells using QIAmp DNA blood mini Kit (Qiagen) according to the manufacturer's instructions. DNA from sorted IgD+ and IgD− DE cells, B_{con} cells and T_{con} cells were used for BCR or TCRBV sequencing as described in text.

High-throughput immune SEQ and data analysis. Analyses of IGHV and TRBV clonotypes were performed on genomic DNA from each sorted cell type using the immunoSEQ platform at survey level resolution (Adaptive Biotechnologies). The immunoSEQ assay combines multiplex PCR with high-throughput sequencing and sophisticated bioinformatics pipeline for CDR3 region analysis (Carlson et al., 2013; DeWitt et al., 2016; Robins et al., 2009). Samples were amplified from 40 ng to 100 ng genomic DNA per sample. Attempts to sequence IgD− from T1D #1 and IgD+ cells from T1D #2 were unsuccessful. TCR and IGH sequences will be available at Adaptive Biotechnologies. Raw ImmunoSeq data from individual samples were processed with ImmunoSeq Analyzer 2.0 software (Adaptive Biotech). Measurement metrics of processed data were exported in the tsv file format and analyzed using the R platform. Clones of uncertain vGene identity or out-of-frame were excluded from downstream analysis. For vGene in each cell type in individual samples, counts of distinct cell clones were obtained by summing the metric of the “estimated number of cell genomes present in the sample”, upon which the corresponding percentages were calculated. The percentage quantification provides a uniform basis for the vGene (VH and Vβ) usages to be fairly and consistently compared across the different cell types and samples, minimizing any effects that could result from sequencing different and very tiny numbers of DE subset cells. Percentages were visualized with bar plots to make straight comparisons of vGene usages between different cell types. The presence or absence of vGenes in the different cell subsets was determined on the basis of the vGene usages. Unique and shared vGenes among different cell subsets were identified and displayed in Venn diagrams using the functions of R Limma package. The vGene mutations are identified based on alignment with the IMGT database, upon which the differences from germ lines are marked, counted and recorded in the column of “vAlignSubstitutionCount” of the Raw ImmunoSeq data tsv spreadsheet. The vGene mutation values were further summed per gene and displayed with a combination of boxplot and scatter plot using R. String searches with the amino acid sequences of the selected clones were performed to determine their presence in individual subjects. A string search of the amino acid sequence of the invariant clonotype, “CARQEDTAMVYYFDYW” (SEQ ID NO:1) was also performed with an R script against a public ImmunoSEQ database3 of 37 million unique BCR sequences of naïve and memory B_{con} cells and that of IBC examined from nPOD Adaptive Immune Repertoire.

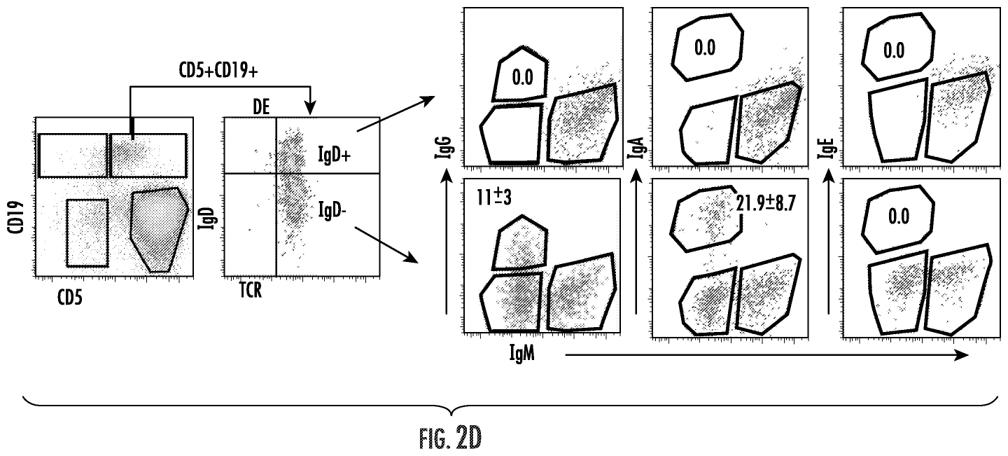

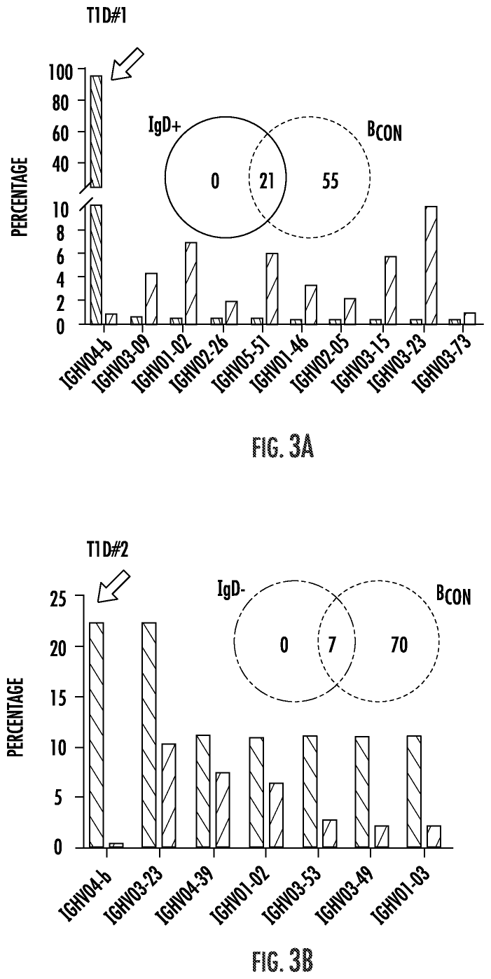

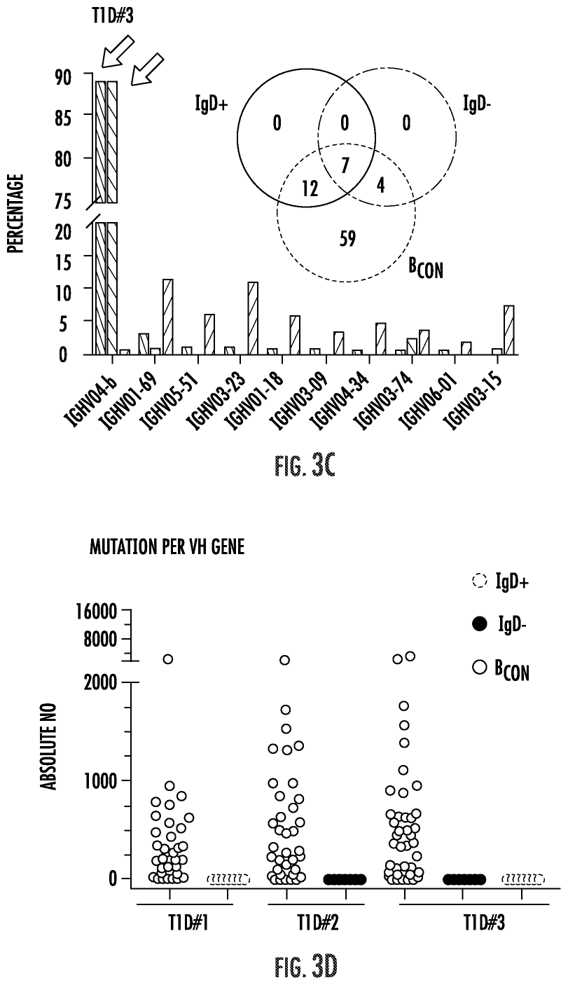

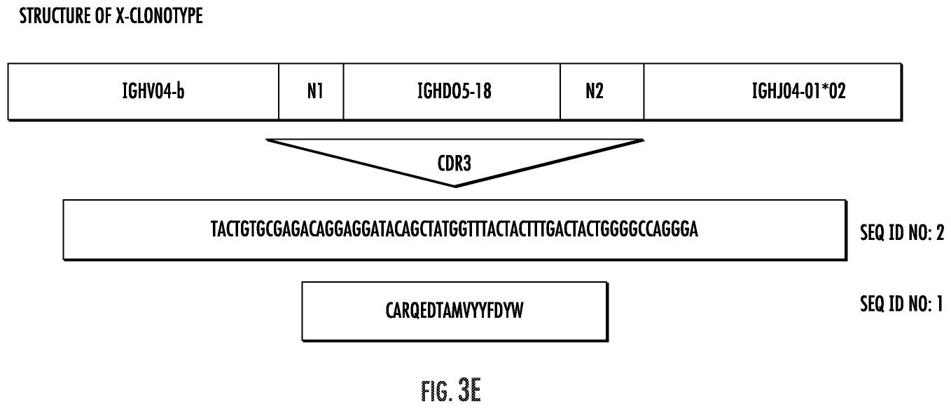

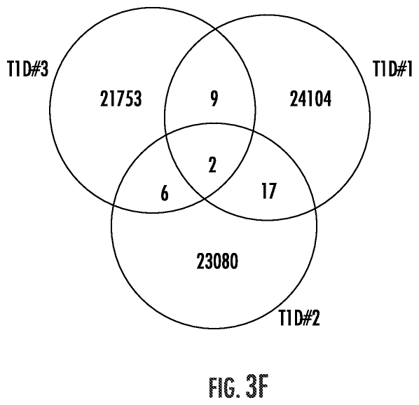

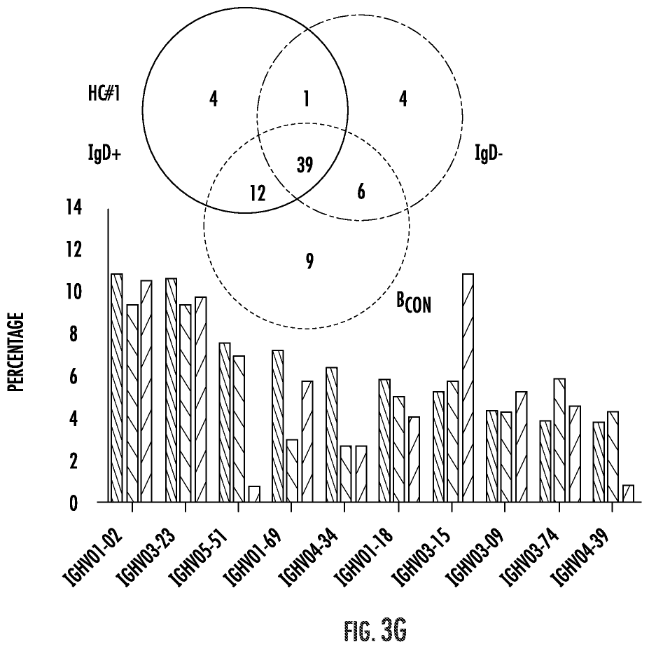

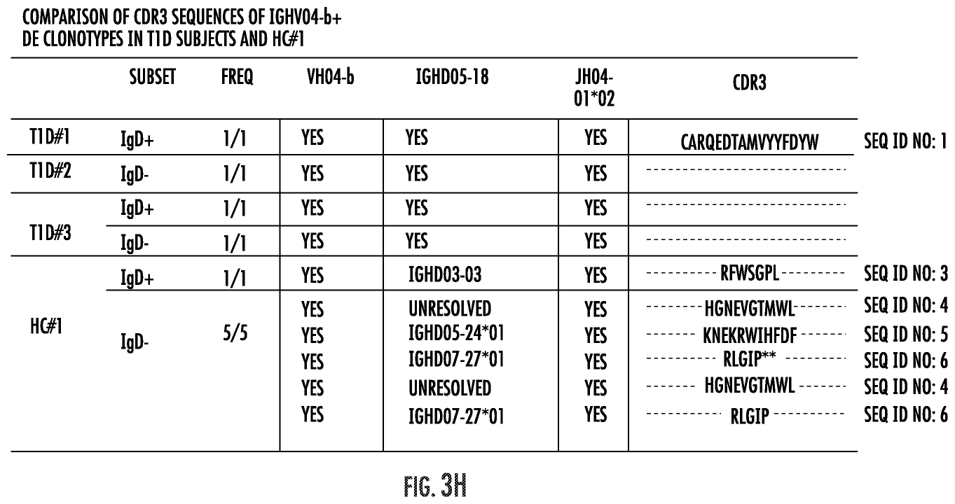

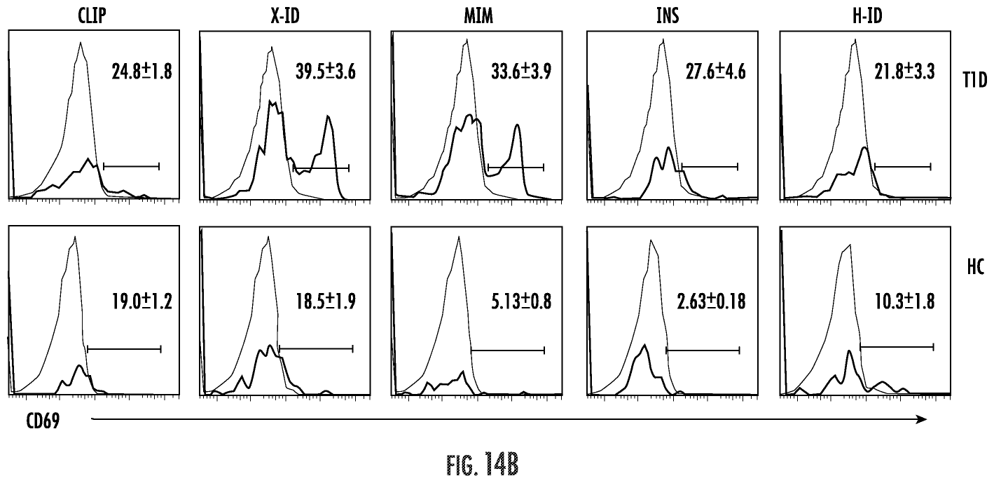

PCR probes for detection of x-clonotype in peripheral blood. To determine whether the x-Id clonotype can be detected in peripheral blood, we designed and used two PCR probes for analysis of PBMCs. In the first probe we used a VH04-b-specific sense primer (5-GCTGGAGTGGATTGGGAGTA-3) (SEQ ID NO:17) paired with antisense primer (CCCAGTAGTCAAAGTAGTAAACCATA) (SEQ ID NO:18) complementary to the entire CDR3 region (see diagram, FIG. 3J). In the second probe, the VH04-b-specific primer was paired with a reverse primer (3-TCCCTGGCCCCAGTAGTCAAAGTAGTA-5) (SEQ ID NO:19) that span JH04 and ended at the N2 region (see diagram, FIG. 14). Briefly, RNA was extracted from fresh PBMCs using the RNeasy blood mini kit (Quigen) and analyzed by NanoDrop (ND-1000 spectrophotometer) to assess purity and measure concentration. Reverse transcription (RT) PCR was performed on approximately 1 μg of purified RNA to prepare cDNA using the RevertAid First Strand cDNA Synthesis Kit (Thermofisher) according to the kit protocol: RNA was incubated with 5× reaction mix, random hexamer primer, and RevertAid M-MuLV RT (200 U/μL) enzyme mix in a final volume of 20 μL at 25° C. for 10 min, followed by 42° C. for 60 min and inactivation at 70° C. for 5 min. Positive (GAPDH-specific primers) and negative (reaction mixture without RT enzymes) control reactions were used to verify specificity of cDNA synthesis. PCR reaction (2 μL cDNA in a total volume of 25 μL prepared using 2× QIAGEN HotStarTaq master mix) was performed under the following conditions: initial denaturation at 95° C. for 3 min, 95° C. for 30 s, 54° C. for 30 s, 40 cycles at 72° C. for 1 min followed by a final extension step at 72° C. for 10 min using a thermocycler (BioRad T100). PCR product was visualized as a band size of 200 bp on 1.2% agarose gel and the band was excised, purified using PCR purification kit (Quigen) and Sanger sequenced at the Johns Hopkins Medical Institute GRCF sequencing core. Sequences were analyzed using the Immunogenetics IMGT/V-QUEST software.

Molecular dynamics simulations. The new peptide system was built from a crystal structure of an insulin B chain epitope bound to HLA-DQ8 (PDB ID 1JK8) (Sharp, 2012). The insulin epitope sequence was mutated to the new peptide epitope sequence using the Mutator plugin from VMD, ensuring the new peptide epitope was in the desired register. The CDR3 epitope from HC #1 was also built from the insulin-bound epitope structure (PDB ID 1JK8), following the same protocol as the new peptide system. The super-agonist system was built from the crystal structure of an insulin mimotope bound to HLA-DQ8 (PDB ID 5UJT) (Wang et al., 2018). For this system, in addition to mutating the epitope to match the super-agonist, both HLA chains α and β were mutated to match the sequence of the HLA in the insulin crystal structure (PDB ID 1JK8). More specifically, besides distal residues, residue 72 C of HLA-α was mutated to Isoleucine to match the 1JK8 HLA sequence. Each system was solvated in a TIP3P water box and then charged, neutralized, and ionized with 100 mM concentration using Na+ and Cl—.

Following system creation, each system underwent at least 20,000 steps of conjugate-gradient minimization to hold protein atoms fixed, followed by at least 10,000 steps of minimization allowing all the atoms to move. The systems were subsequently equilibrated for 1 ns at 310K using a 2 fs timestep. Production MD simulations were run for 500 ns using a 2 fs timestep. A Langevin thermostat maintained the temperature at 310K. The CHARMM36 force field (Best et al., 2012) was used for protein parameters. The Particle Mesh Ewald (PME) method was used to compute long-range electrostatics with the electrostatics and van der Waals cutoff of 12 Å. All simulations were run using NAMD2.11. For the MD simulations, only the last 250 ns were used for analysis, dividing the trajectory into 5 parts.

The contact area was computed using solvent accessible surface area (SASA) calculation in Gromacs tools with a water radius of 1.4 Å. Van der Waals interaction energy was computed using NAMDEnergy. The electrostatics energy was biased due to the absence of solvent screening and was left out of the interaction energy. The RMSD and RMSF were computed using Gromacs tools. Averages and error bars for contact area, interaction energy, and RMSF were computed by taking the last half (250 ns) of the MD simulations and dividing them into 5 sections with 50 ns each and taking the average of each section as a measurement in the sample. Error bars shown are standard error.

Free energy perturbation. Binding affinity was calculated via the free energy perturbation (FEP) method. The final structures of the production MD simulations were selected for FEP computation. We computed free energy perturbation calculations for the bound (HLA+epitope) and free states (epitope only) with 6 replicas for each calculation. Due to the extensive sequence differences between epitopes, we mutated the epitopes to a neutral, intermediate sequence of polyglycine the length of the epitope. The dual topology was implemented using the Mutator plugin from VMD. Each system was slowly mutated from the epitope to polyglycine using λ increments of maximum 0.04 with smaller increments towards the ends, totaling at least 34 FEP windows for each system. Each FEP window was run for 1 ns, leading to well over 800 ns simulation (6 replicas×34 windows×2 states (complex+free)×2 epitopes). Electrostatics was switched on starting at λ=0.1. Convergence at each window was assessed by comparing values across replicas. NAMD2.11 with the CHARMM36 protein force field and TIP3P water model were used for FEP calculations, matching the MD work. From observing the FEP trajectories, the polyglycines do not shift registers but maintain the starting register of the epitope. Free energy error bars are standard errors.

Analysis of x-Id peptide binding to DQ8 using gentle SDS-PAGE assay. Gentle SDS-PAGE was used to assess formation of stable complexes between peptides and HLA-DQ8 molecules as previously described (Kim et al., 2013; Sadegh-Nasseri and Germain, 1991). Briefly, 0.5 μM of HLA-DQ8 monomers (provided by NIH tetramer core facility) were treated with thrombin to cleave and remove CLIP peptide. Empty monomers were incubated in the absence or presence of 100 μM of indicated peptides (x-Id, TP-Id, mimotope (R22E), native insulin B:9-23, and h-Id) at 37° C. for 72 h in citrate phosphate buffer, pH 5.5 with 1 mM PMSF and 0.025% NaN3. Reactions were neutralized, mixed with equal volumes of SDS-PAGE sample buffer containing 0.1% SDS (final concentration) and placed for 15 min at room temperature and run on 10% PAGE gels and silver-stained using a standard protocol. To assess stability some samples were boiled for 3 min, which resulted in degradation of complexes (data not shown).

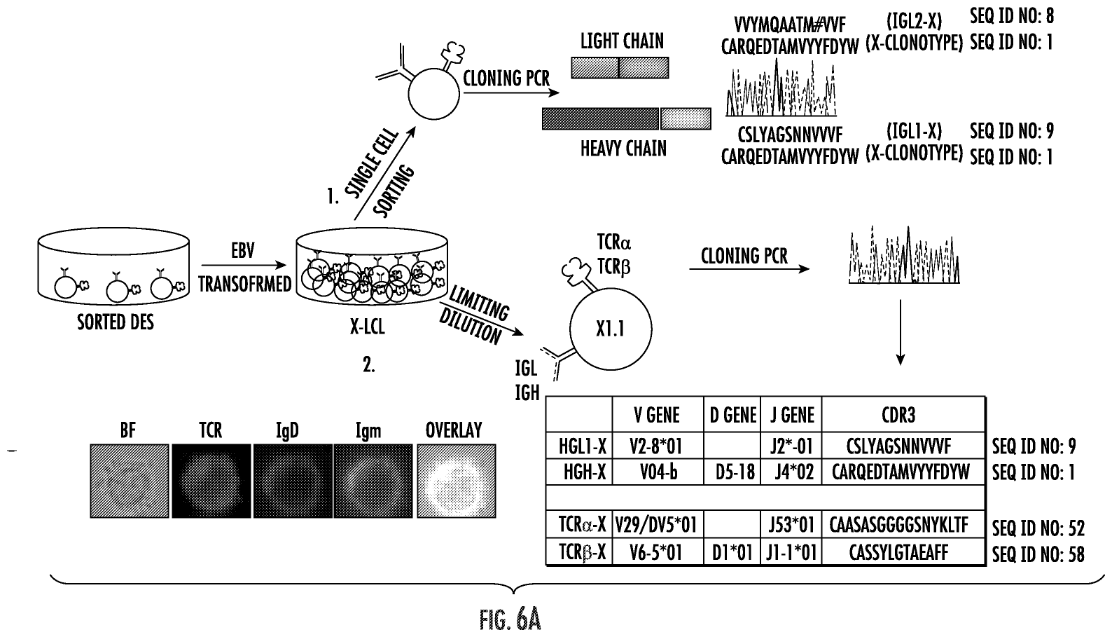

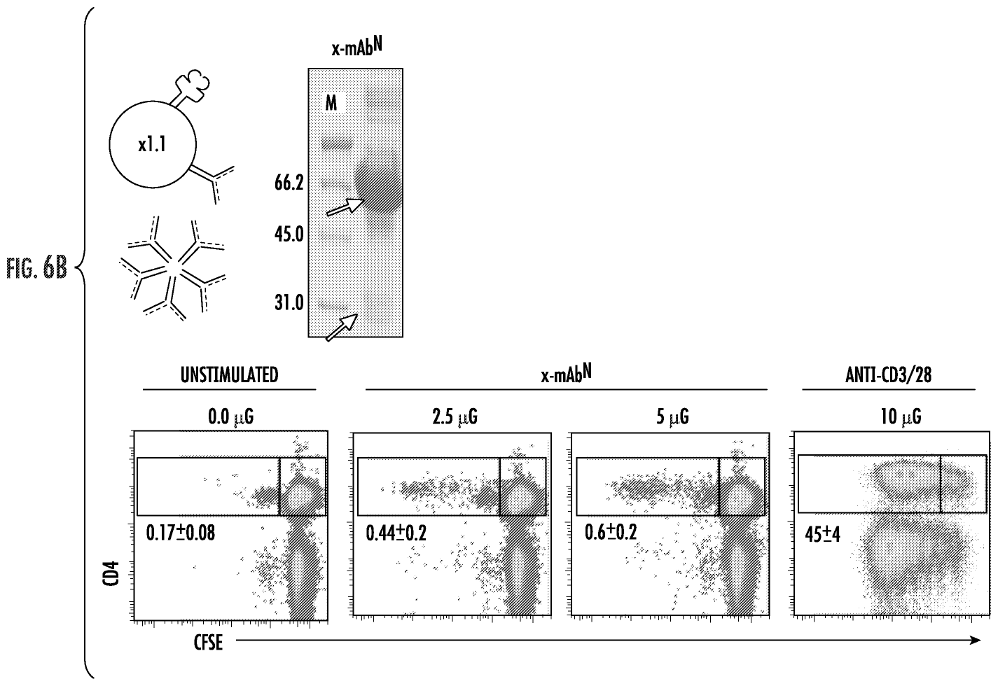

Generation of EBV-immortalized DE x1.1 clone. To generate immortalized DE cells, we sorted IgD+ DE cells from freshly isolated PBMCs using a FACSAria II using described strategy (FIG. 6A). Sorted cells were seeded at 10, 25, 50 or 100 cells per well of 96-well microplates that had been coated 24 h earlier with irradiated fibroblasts (ATCC® 55-X™) Cultures were pulsed with 2.5 μg/ml CpG ODN 2006 (ODN7909) and EBV supernatant stock from B95-8 cells (ATCC® VR-1492) according to the method described by Caputo et al. (Caputo and Flytzanis, 1991). Cultures were maintained by replacing half of culture medium with fresh medium every 5 to 7 days. Immortalized cells were visible after 8 days in cultures seeded with 50 or 100 DE cells. We selected on lymphoblastoid cell line (hereafter referred to as x-LCL) for subsequent analysis. In one set of experiments, we sorted single cells from x-LCL and examined for expression of Ig heavy and light chains. In a second set, we used x-LCL to generate x1.1 clone by limiting dilution (0.3 cell/well) as described (Hamad et al., 1994). Cells of the x1.1 clone were used for analysis of BCR and TCR and spontaneous antibody production.

Analysis of x1.1 clone for coexpression of BCR and TCRαβ. We used two approaches to ensure single cell clonality of immortalized DE cell populations, outgrown monoclonal DE cells were sorted on 96 well microplates using a FACSAria II (BD Biosciences, Bedford, Mass.) as described above containing RNA catch buffer (Smith et al., 2009).

Cloning and expression of BCR from x1.1 clone and fresh single DE cells. We used the same protocol that was developed by Smith et al. (Smith et al., 2009) for analysis of BCR expression from single cells of the x1.1 clone and freshly sorted single DE cells. Briefly, individual cells were sorted into wells of 96-well PCR plate loaded with catch buffer containing RNase inhibitor to perform RT-PCR using OneStep RT-PCR Kit (Qiagen). Two primers were utilized to amplify all VH4 gene family members and 8 primers for amplification of genes encoding lambda chain. Cloning PCR of the heavy chain was performed using primers that incorporate the cloning restriction sites and place VDJ heavy chain and constant region genes in frame within the cloning vector (AbVec-hIgG1). Cloning PCR products were purified using Monarch PCR & DNA Cleanup Kit (New England, BioLabs) and visualization as a band of approximately 400 bp in 1.5% agarose gel. Insert and vector were digested with AgeI and SalI and purified as described above. A three-fold molar excess of insert to vector were used to transform DH5a cells. Positive colonies were picked, cultured and plasmid extracted by QIAprep Spin Miniprep Kit (Qiagen) followed by sequencing using the AbVec primer. The lambda chain was cloned using the same procedure except that insert and vector were digested with AgeI and XhoI and cloned into AbVec-Complete sequences of the variable regions were used to identify VDJ usage and CDR3 by IMGTV-Quest.

Expression and analysis of recombinant x-mAbR from single DE cells. Following purification and digestion, amplified cDNAs of the antibody variable genes from single cells were cloned into expression vectors containing human IgG, and Igλ constant regions (AbVec-hIgG1, AbVec-Igλ) as previously described (Smith et al., 2009). AbVec-hIgG1 containing the heavy- and AbVec-Igλ containing light-chain Ig genes were co-transfected into the 293A cell line using polyplus jet-prime transfection (who) and manufacturer's instruction. Transfected 293A cells were allowed to secrete antibodies in serum-free basal media for 4 to 5 days and mAbR was purified using immobilized protein A columns (Pierce). Antibody expression and purity was verified by SDS-PAGE, and purified antibody concentrations were determined using the EZQ Protein Quantitation Kit (Invitrogen).

Cloning of α and β chains of TCR from the x1.1 clone. The genes for TCRα and TCRβ chains were cloned using a modified version of the method described by Eugster et al. (Eugster et al., 2013). Briefly, total RNA was isolated from cells of the x1.1 clone using RNA extraction kit (Biolabs). cDNA was prepared and mixed with degenerate primers for the α and β chains using OneStep RT-PCR Kit (Qiagen) and used to amplify the α and β chains by nested-PCR using specific primers for the α chain and β chains, separately. Amplified products were visualized on 1.5% agarose gel and cloned into pGEM®-T Easy vector (Promega). DNA was extracted using plasmid extraction kit (Qiagen) and Sanger sequenced using the M1 primer (Eugster et al., 2013). Complete sequences of the variable regions were used to identify VDJ usage and CDR3 by IMGTV-Quest.

Characterization of the x-mAbN produced by x1.1 clone. Cells of x1.1 clones were expanded in complete media for 3-4 days, washed with PBS and cultured in basal media (Star method) for five days. Secreted mAbN was detected in supernatants by using SDS-PAGE. Isotype of secreted mAb was determined as IgM using Pro-Detect™ Rapid Antibody Isotyping Assay human Kit (Thermo Fisher). Antibody concentration was determined using the EZQ Protein Quantitation Kit (Invitrogen)

Tetramer preparation and staining. We made three DQ8 tetramers using biotinylated HLA-DQ monomer provided by (NIH Tetramer Core Facility) using stablished method (Crawford et al., 2011). One tetramer is made of HLA-DQ with x-Id peptide (x-Id tet), one complexed with insulin mimotope (mim-tet) and the third one complexed with CLIP (CLIP-tet). Clip was removed using thrombin and empty monomers were loaded with 0.2 mg/ml of either x-Id or Insulin-mimotope peptide. Loading was conducted at 37° C. for 72 h in the presence of 2.5 mg/ml (0.25%) n-octyl-β-Dglucopyranoside and 1 mM Pefabloc SC (Sigma-Aldrich). Peptide-loaded HLA-DQ monomers were tetramerized with PE-conjugated streptavidin (eBioscience) at a molar ratio of 1:4, respectively. HLA-DQ/CLIP monomer was tetramerized as control negative in staining. The successful formation of tetramer complexes were verified by gentle SDS-PAGE. Tetramer staining was performed incubating PBMCs with 2 μg/ml HLA class II tetramer for 1 hr at room temperature in FACS buffer. Antibody specific for surface CD4, TCR and CD19 have been used and samples were acquired. The data was analyzed as described above (Dai et al., 2015).

Quantification and statistical analysis. Description of experimental replicates and sample sizes are describe in the figures legends. Statistical significance of the results was performed using Prism 6 (GraphPad Software). Analysis was performed using independent samples t-test or a paired sample student t-test as appropriate. The results were expressed as the mean±SEM unless stated otherwise. p<0.05 was considered as statistically significant.

Data and software availability. RNA-seq, and DNA-seq data reported in this paper will be deposited at GenBank upon acceptance of the manuscript for publication.

### Results

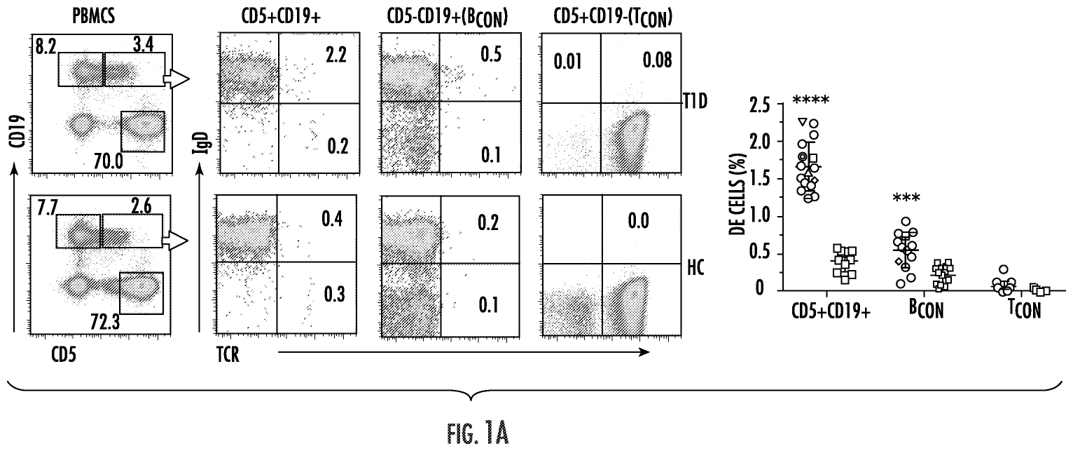

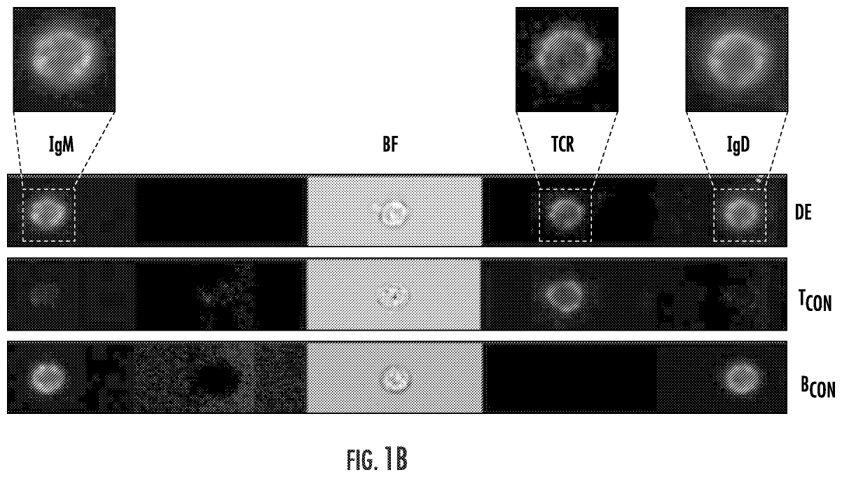

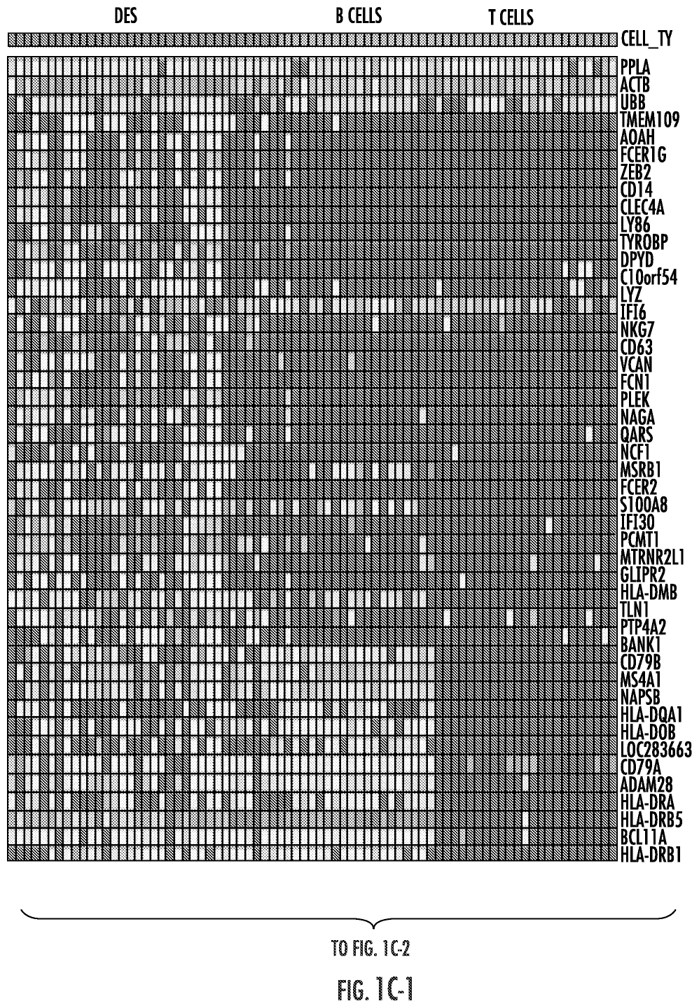

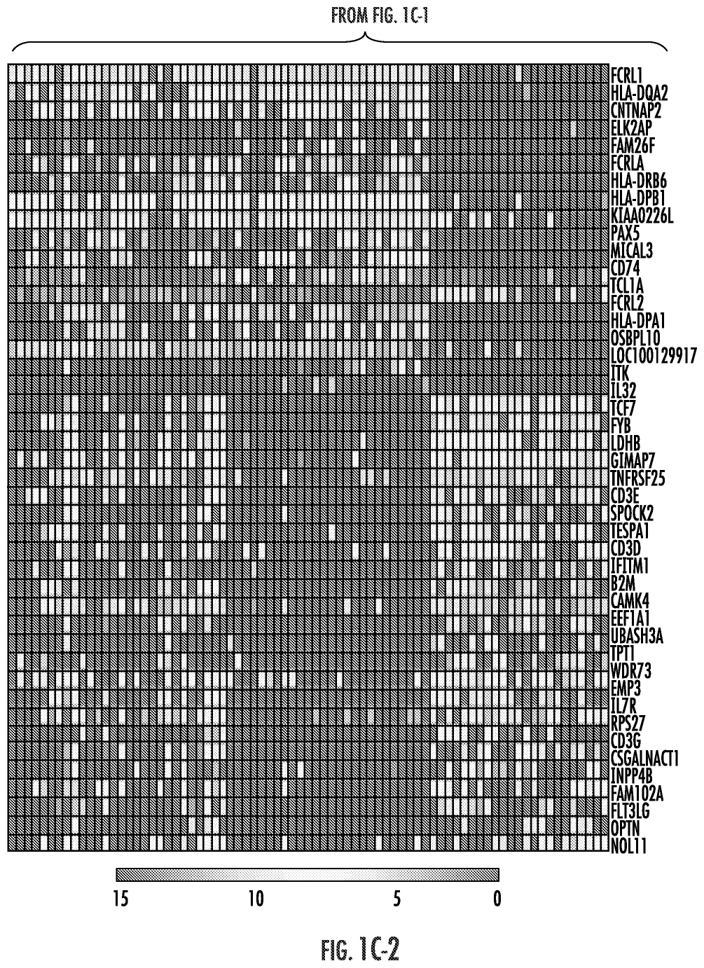

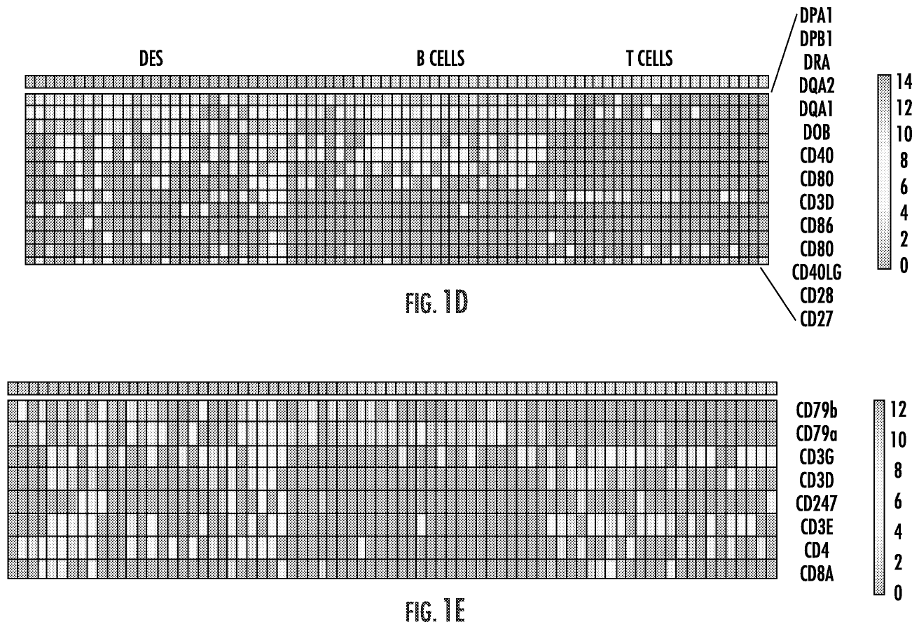

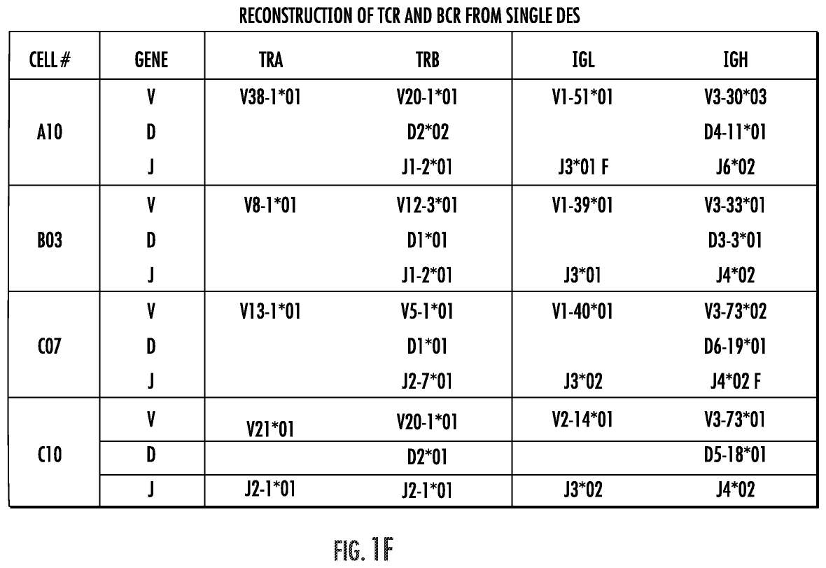

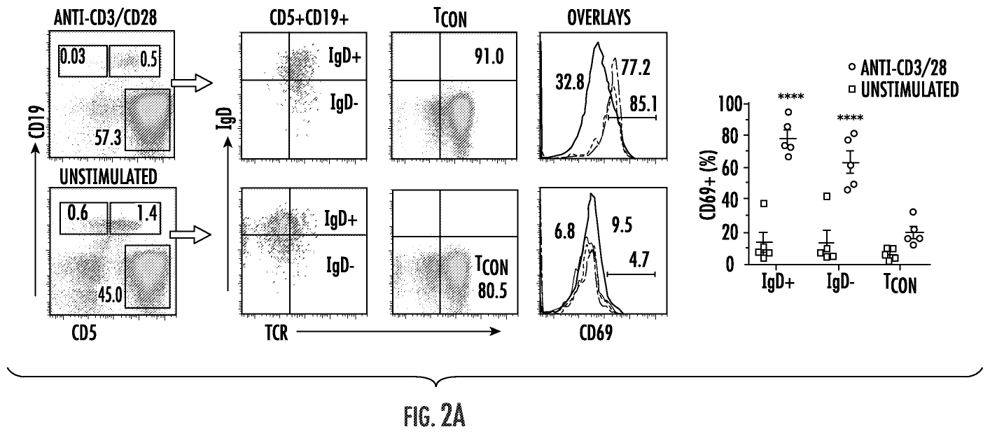

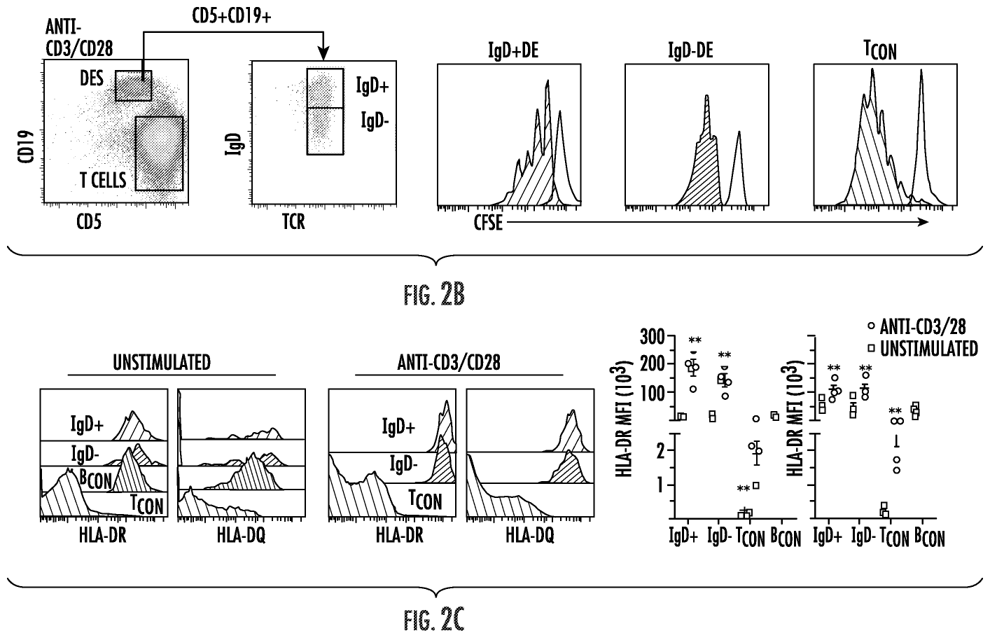

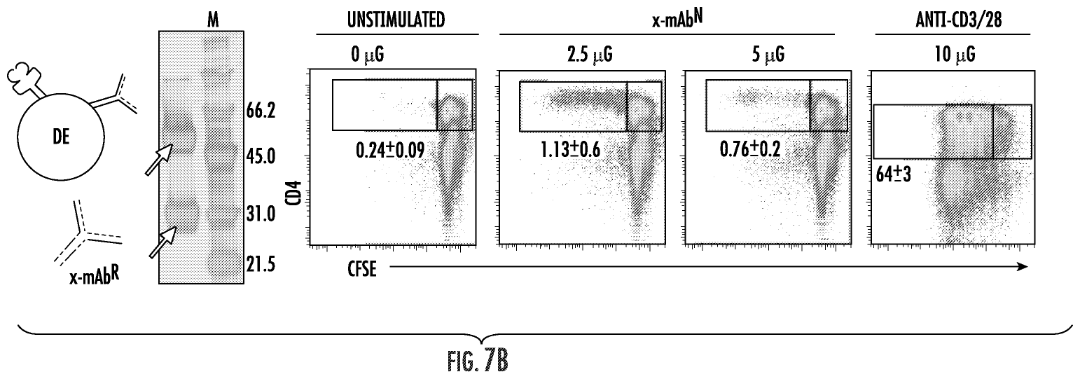

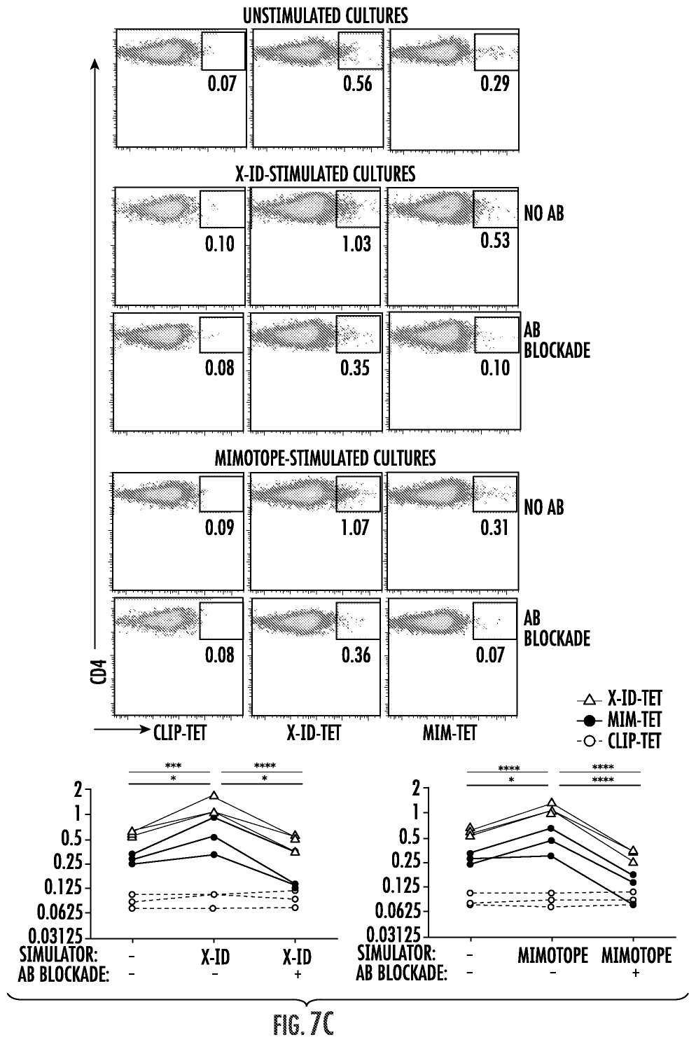

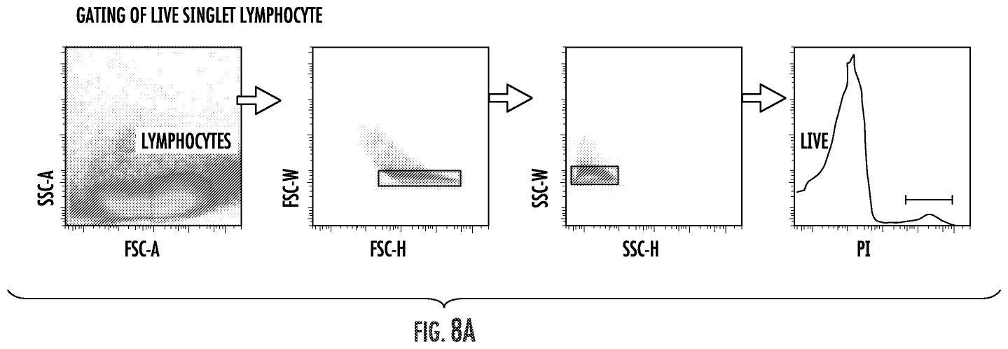

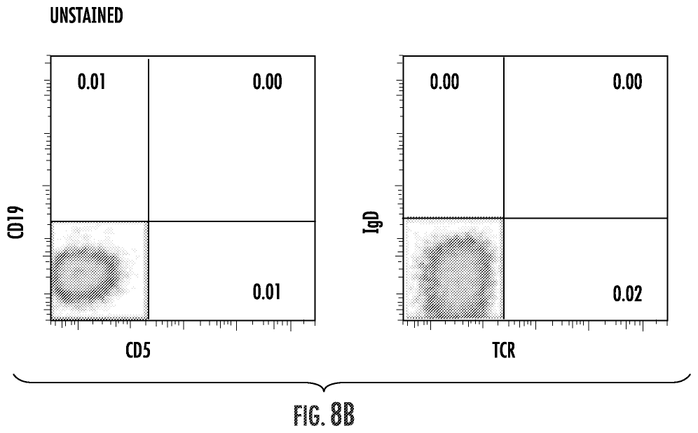

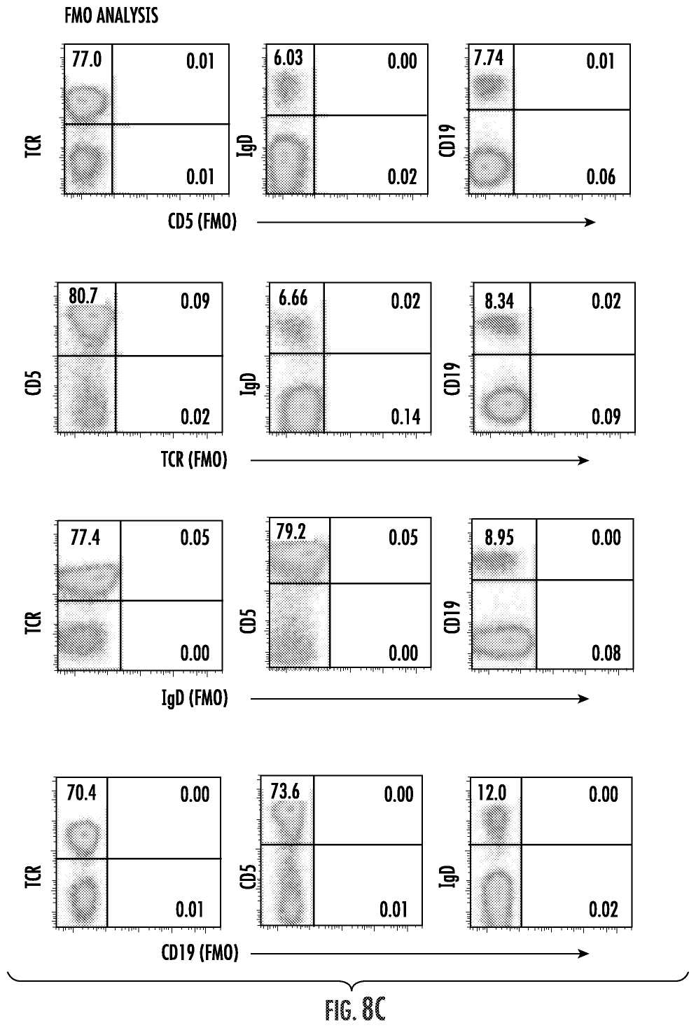

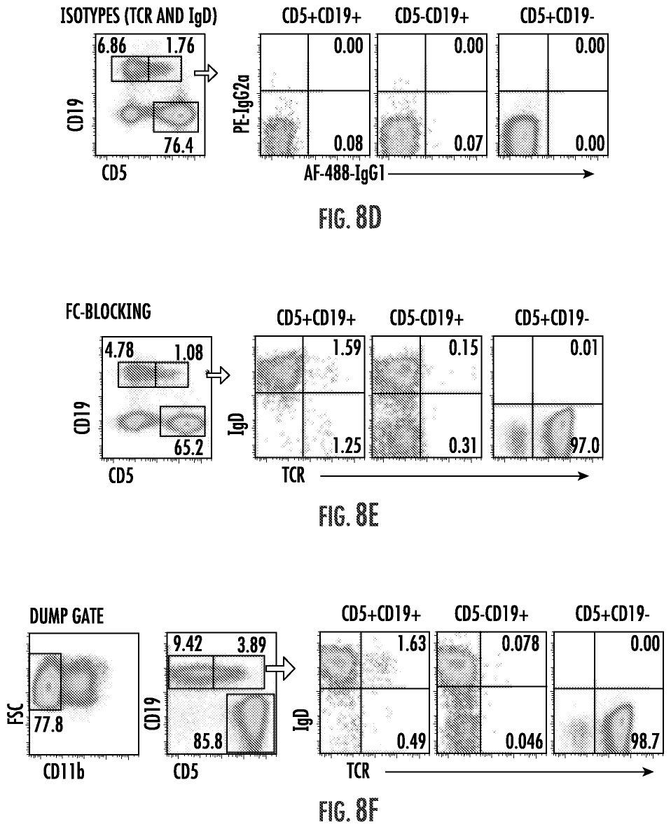

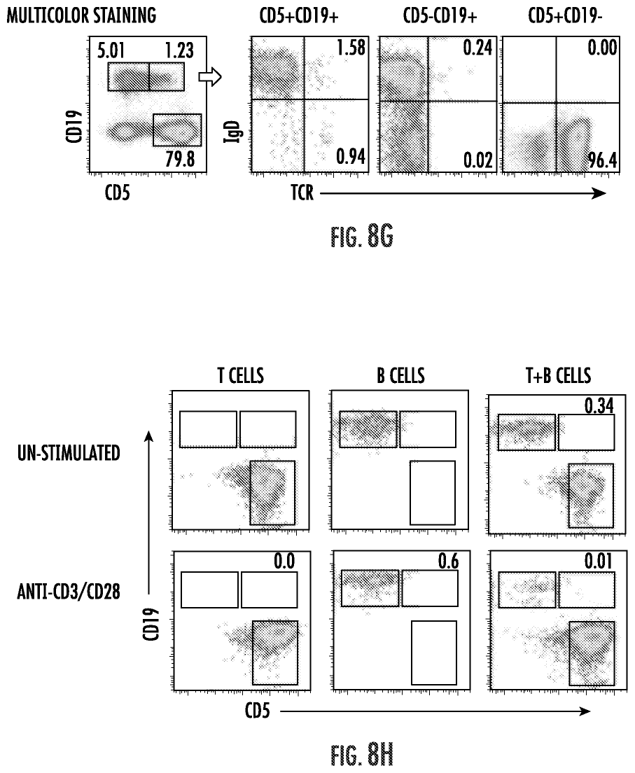

A rare subset of lymphocytes coexpresses T and B cell lineage markers and expands in T1D. We identified a rare population of lymphocytes that coexpresses the BCR and TCR and found predominantly among the CD5+ CD19+ population in peripheral blood (FIGS. 1A and 8 for gating strategy). The majority of these dual expressers (hereafter referred to as DEs) expressed IgD/IgM and were phenotypically identified as CD5+ CD19+ TCRβ+ IgD+ cells (FIG. 1A). A minor subset of CD5+ CD19+ TCRβ+ were IgD−, but expressed IgG, IgA or IgM and could thus be class-switched DEs (see FIG. 2D below). Few DEs were present among CD5− CD19+ B_{con} cells and were not analyzed further. Instead, we focused on IgD+ and IgD− DEs found in the CD5+ CD19+ compartment. DEs were significantly more frequent in T1D subjects than in healthy controls, HCs (FIG. 1A and see subject characteristics in Table 1, not shown). We visualized coexpression of the IgD, IgM and TCR in DEs at the single cell level using flow cytometric imaging, AMNIS (FIG. 1B). Thus, although rare, DEs could be pathophysiologically important due to their expansion in T1D. To investigate the dual phenotype of DEs at single cell resolution, we examined their transcriptomes using single cell RNA sequencing (scRNA-seq). We sorted individual DEs, B_{con} and T_{con} cells from PBMCs of T1D #1 and analyzed their transcriptomes using the plate-based SMART-seq2 protocol (Tirosh and Suva, 2018; Tirosh et al., 2016). A total of 77 cells (34 DEs, 20 B_{con}, and 23 T_{con}) passed quality control based on the expression of at least two out of three housekeeping genes (FIG. 1C). Results show the top 30 distinctively differentially expressed genes by DEs compared to T_{con} or B_{con} cells. Simultaneously, DEs expressed the top 30 differentially expressed genes between B_{con} and T_{con} cells. The results show the crossover phenotype of DEs at single cell resolution. Expression of unique sets of genes by DEs points to a complex transcriptome worthy of detailed future investigation.

We highlighted shared expression of selected lineage markers of B and T cells by DEs (FIGS. 1D and 1E). These were also visualized at the protein level using FACS and AMNIS (FIGS. 1B and 2C and 9 and 10). We also analyzed DEs for the invariant components of BCR (CD79a, and CD79b) and TCR (CD3γ, CD3ε, CD3ζ, and CD247) that are responsible for transducing activation signals. In line with our FACS, AMNIS and functional data, DEs shared expression of CD79a, and CD79b with B_{con} cells and CD3γ, CDε, CD3δ and CD247 (CD3ζ) with T_{con} cells (FIG. 1E).

Finally, we used the bioinformatics BASIC (BCR assembly from single cells) software to reconstruct recombined BCR and TCR genes expressed in DEs using B_{con} and T_{con} cells as controls. We detected contigs of both BCR and TCR in DEs and as expected we detected contigs of BCR genes in B_{con} and those of TCR genes in T_{con} cells (Table 2, not shown). We used the IMGT/V-QUEST software and examined reconstructed sequences for V(D)J usage. Many single DEs successfully reconstructed at least one (22 cells) or both (18 cells) BCR chains (Table 2A, not shown). In addition, several (8 cells) had fully assembled TCRβ variable (Table 2B, not shown). Importantly, four individual DEs coexpressed fully assembled BCRs (heavy and light chains) together with fully assembled TCRβ chain with TCRVα (FIG. 1F). As specificity controls, there were no assembled TCR chains in B_{con} cells or BCR chains in T_{con} cells. Collectively, these results provide proof of principle of the existence of lymphocytes with a hybrid phenotype of T and B cells.

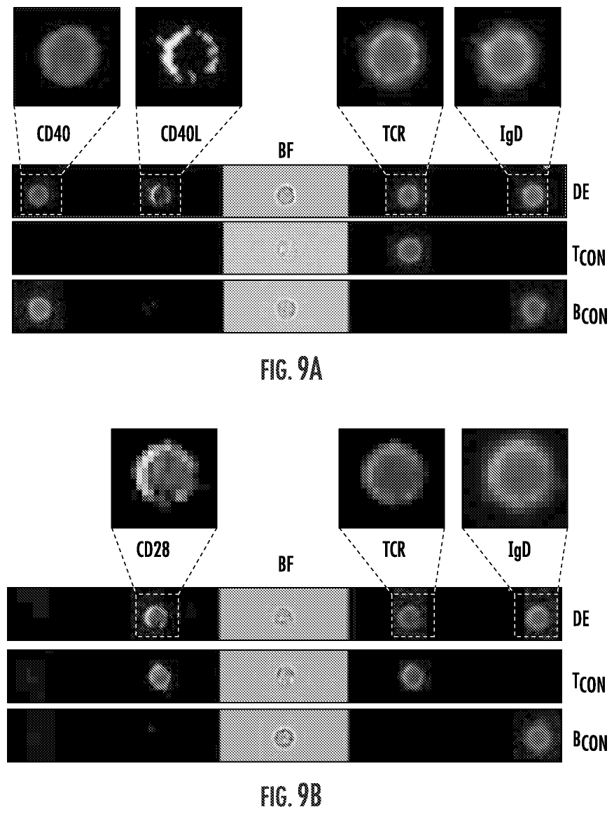

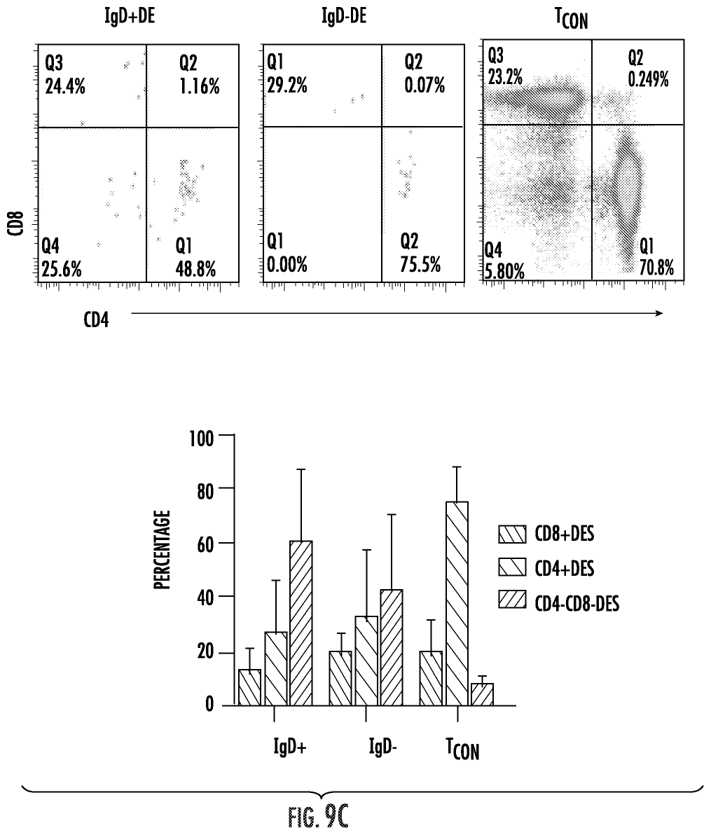

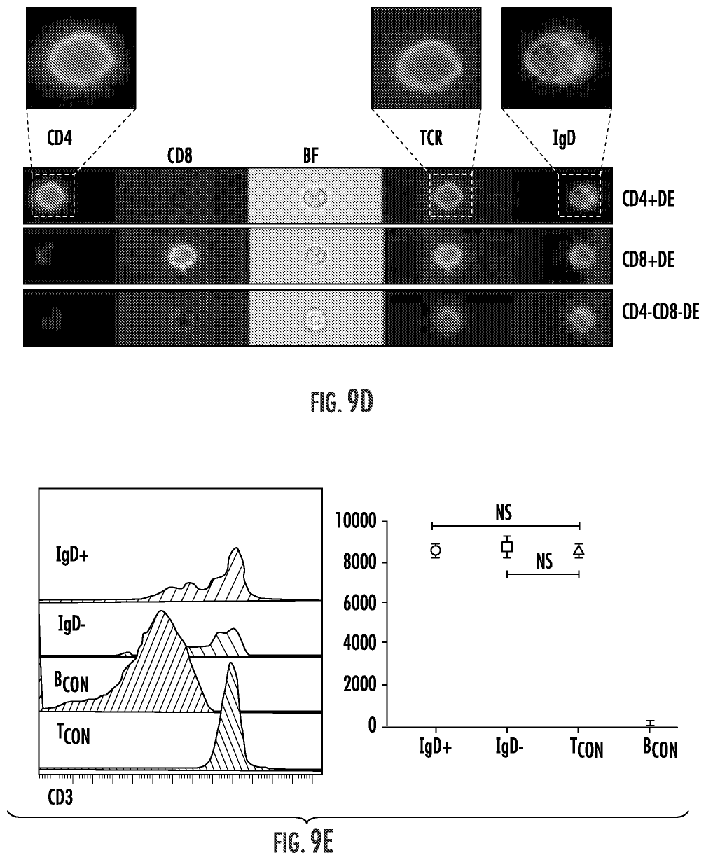

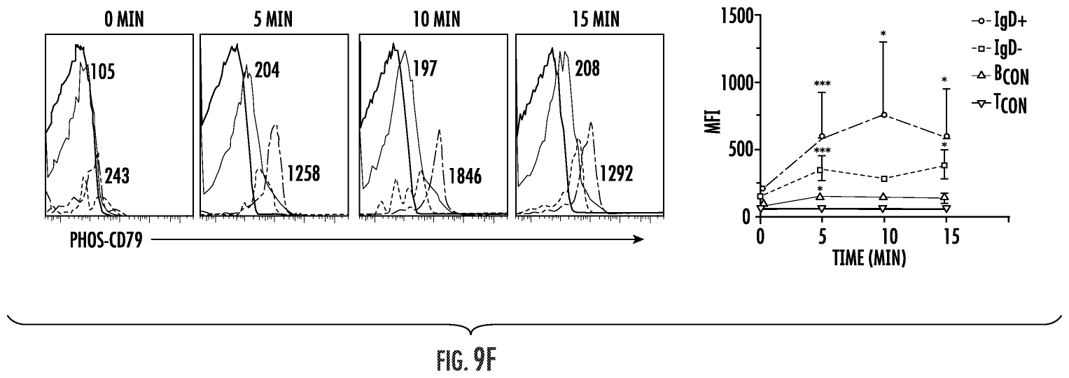

TCR-activated DEs maintain their dual phenotype and upregulate MHC and B cell costimulatory molecules. Next, we examined functionality of TCRs expressed on DEs and phenotypic and functional consequences of their crosslinking. Consistent with scRNA-seq analysis, DEs expressed the CD3c signaling subunit (FIG. 9E), suggesting a functional TCR/CD3 complex. To test this notion, we activated PBMCs with anti-CD3/CD28 for 7 d and analyzed different subsets for CD69 upregulation. Because of the rarity of DEs in HCs, we did our experiments using PBMCs from T1D subjects unless stated otherwise. Analysis of activated cultures show that CD19+ CD5− B_{con} cells regressed to a minor component of lymphocytes and could not be properly analyzed for CD69 expression, consistent with being bystanders. Consequently, remaining CD19+ cells were present within the CD5 intermediate gate (87.3%±11.7; n=5), expressed TCR and included IgD+ and IgD− DE (FIG. 2A). TCR stimulation led to significant upregulation of CD69 by IgD+ and IgD− DEs as compared to DEs in control cultures (FIG. 2A). T_{con} cells which made the bulk of cultures also upregulated CD69 but significantly less than DEs. Thus, TCRs on DEs are not only functional molecules, but are highly responsive to TCR crosslinking.

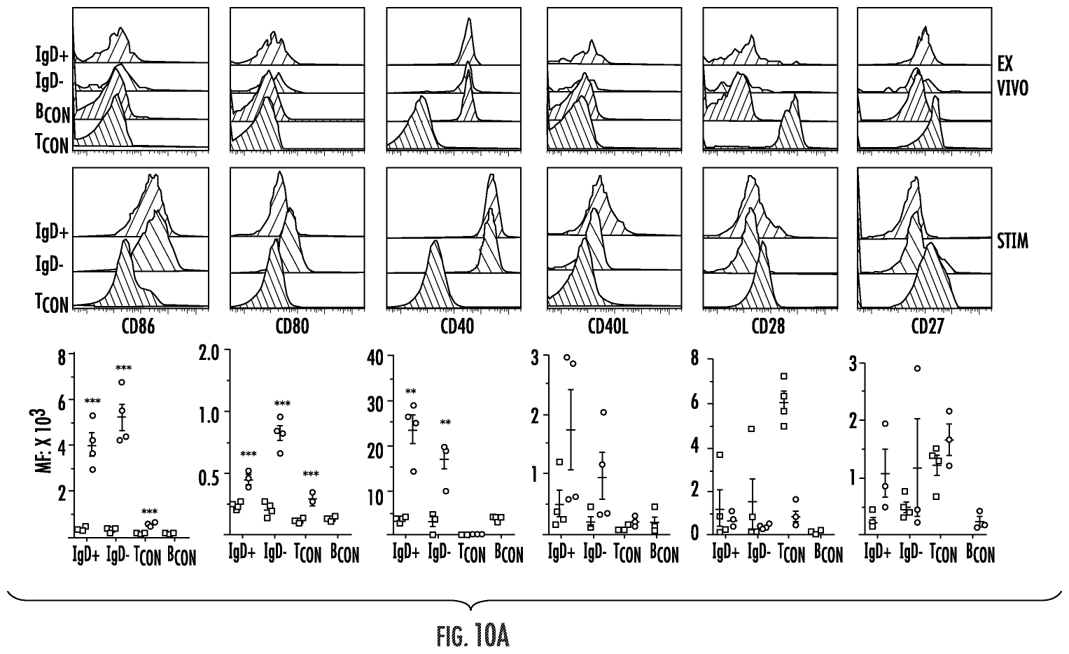

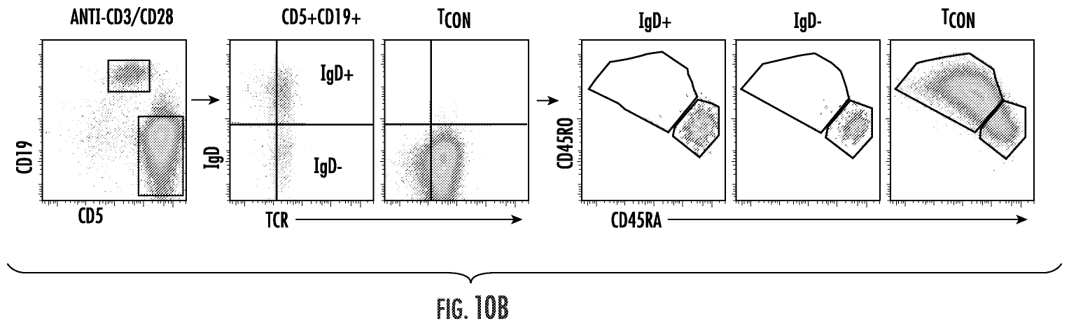

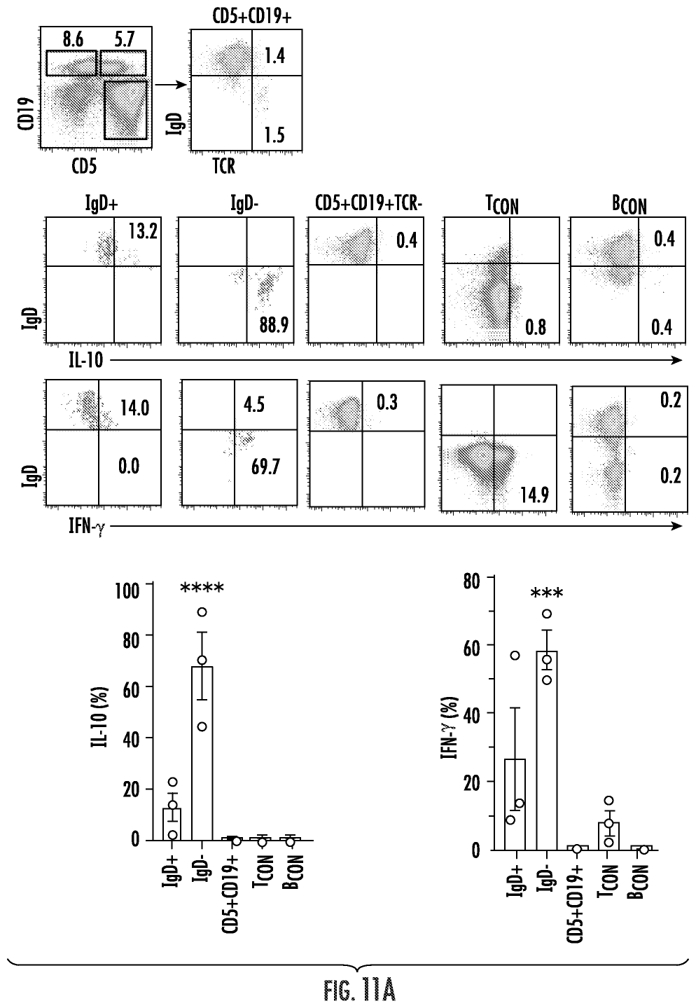

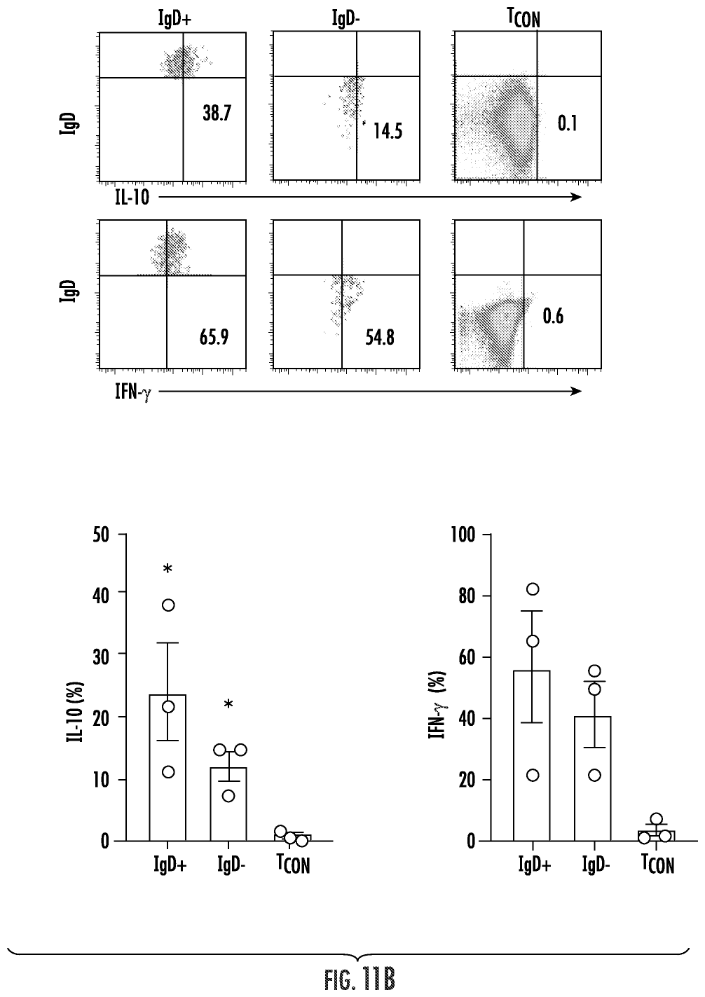

In a second set of experiments, we used proliferation as a readout. We stimulated PBMCs with anti-CD3/CD28 and visualized proliferation using the CFSE dilution assay (FIG. 2B). Consistent with the above results, B_{con} cells regressed and most remaining CD19+ cells in activated cultures expressed an intermediate level of CD5. Both IgD+ and IgD− DEs, similar to T_{con} cells, divided robustly in response to anti-CD3/CD28 stimulation as indicated by CFSE dilution (FIG. 2B). As shown above by scRNA-seq analysis, DE expressed transcripts for MHC molecules and key costimulatory molecules. We thus verified expression of these molecules at the protein level and examined the modulatory effects of TCR stimulation on HLA (FIG. 2C) and indicated costimulatory molecules (see FIG. 10A). FACS analysis confirmed that DEs expressed HLA-DR (DR) and HLA-DQ (DQ) molecules and several pan-B and -T cell markers. Anti-CD3/CD28 stimulation led to significant upregulation of DR and DQ molecules (FIG. 2C) and pan-B cell costimulatory markers, whereas pan-T cell markers did not significantly change (FIG. 10A). Most IgD+ DEs cells maintained coexpression of IgM and none switched to IgA, IgG and IgD− DEs remained mixtures of IgG+, IgA+ and IgM+ cells and no IgE+ DE cells were detected (FIG. 2D). Differential expression of Ig isotypes by DEs shows that they do not suffer from generalized dysregulated gene expression. In addition, activated DEs expressed CD45RA and none expressed CD45RO (FIG. 10B). Likewise, consistent with transcriptome analysis, DEs differentially expressed CD4 and CD8 coreceptors while some were CD4 and CD8 double negative (FIGS. 9C and 9D). It is also noteworthy that, DEs particularly IgD− cells produced IL-10 and IFN-γ when stimulated via PMA/ionomcyin or with anti-CD3/CD28 (see FIG. 11). These results indicate that TCRs and BCRs remain stably coexpressed on DEs after TCR-mediated expansion.

We also examined the functionality of BCRs expressed on DEs. FACS analysis confirmed expression of CD79a (Igα) and CD79b (Igβ) by DEs, indicating a functional receptor (Luisiri et al., 1996; Reth, 1992; Yu and Chang, 1992). To test this possibility, we determined whether crosslinking of IgM induces phosphorylation of CD79a in DEs. This experiment was possible because almost all IgD+ DEs and a fraction of IgD− DEs were IgM+ (FIG. 2D). We stimulated PBMCs with F(ab)2 goat anti-human IgM and measured CD79a phosphorylation at indicated time points. Anti-IgM stimulation led to extended phosphorylation of CD79a in IgD+ DE cells (FIG. 9F) and to a lesser extent in IgD− DEs (expected given that many IgD− DEs expressed IgG or IgA (see FIG. 2D). On the other hand, B_{con} cells transiently, but significantly phosphorylated CD79a. As a specificity control, T_{con} cells did not show any signal in response to anti-IgM stimulation. These results show that BCR complexes expressed by DEs are functional molecules.

Analysis of TCR and BCR repertoires of DE cells. Next, we sorted and analyzed TCRβ chain (TRBV) and Ig heavy chain (IGHV) repertoires of DEs and compared to their conventional counterparts using genomic DNA and high-throughput ImmunoSEQ (Adaptive Biotech). This analysis was important to determine clonality of DEs and to rule out unforeseen artifacts at the protein level such as the transfer of one plasma membrane protein to another [i.e., trogocytosis (LeMaoult et al., 2007)].

Restricted TCRVβ usage by DE cells. We first analyzed TRBV repertoires of eight DE samples (four IgD+ and four IgD− subsets) from three T1D subjects (T1D #1, #4 and #5). The subjects were unrelated and sequenced at different points in time. The sorting strategy and purity of sorted subsets are shown (FIGS. 12A and 12B). DEs exhibited restricted TCRVβ usage (FIGS. 12C and 12E and Tables 3A, 3B and 3C (Tables not shown)). In general, the IgD+ clonotypes used between 18 to 31 Vβs, the IgD− clonotypes used 5 to 29 Vβs, whereas T_{con} cells used almost all 55 Vβ genes. The paucity of DE cells in HCs did not allow sorting and deep sequencing except from one donor (HC #1) who expressed the DQ2 risk allele, but was negative for islet autoantibodies, IAAs (Table 1C, not shown). We analyzed both IgD+ and IgD− DE cells from HC #1. The IgD+ subset in HC #1 used only 7 Vβs and the IgD− subset used 5 Vβs as compared to usage of 55 Vβ genes used by T_{con} cells (FIG. 12F, Venn diagram, Table 3D, not shown). The skewed TCR repertoires of DEs provides another line of evidence of their distinctiveness. The results also argue against the possibility that TCRs detected on the surface of DEs were derived from T_{con} cells that had been conjugated with B_{con} cells because in such a case, the Vβ usage should reflect the diverse repertoire of T_{con} cells. It is also noteworthy that conjugates are usually short-lived and occur in secondary lymphoid organs, not peripheral blood (Okada et al., 2005). Thus, DEs, unlike T_{con} cells in the same subjects, have a skewed TCR repertoire with limited Vβ usage.

Analyses of IGHV repertoires of DEs identify a public dominant clonotype in T1D subjects. Next, we analyzed the IGHV repertoires of IgD+ and IgD− DEs and B_{con} cells from three T1D (#1, #2, #3) subjects using the sorting strategy described above (FIGS. 12A and 12B). We obtained results from four out of six DE samples: IgD+ cells from T1D #1, the IgD− cells from T1D #2, and both the IgD+ and IgD− cells from T1D #3. We obtained IGHV sequences of B_{con} cells from each subject. There was predominant usage of the IGHV04-b gene recently named IGHV4-38-2 (Watson et al., 2013) by DEs in the three T1D subjects. It was used by 95% of IgD+ cells in T1D #1, 22% (top clonotype) of IgD− cells in T1D #2, and 88% of IgD+ and IgD− cells in T1D #3 (FIGS. 3A, 3B and 3C). In contrast, the VH04-b gene was used by less than 1% of B_{con} cells and ranked by usage number 27/76, 31/77 and 28/82 in the three subjects, respectively (Table 4, not shown). Moreover, there was no significant overlap in VH usage by DEs and B_{con} cells. In fact, the top 10 VH genes used by B_{con} cells (particularly T1D #1 and T1D #3) were either entirely absent or constituted minor components of DE repertoires (FIGS. 12G, 12H, 12I and 12J). A complete list of the VH genes used by DE cells and B_{con} cells in the three subjects is shown (Table 4, not shown). Furthermore, the VH genes used by DEs, unlike their counterparts on B_{con} cells, were mainly of germline configuration (FIG. 3D). The distinct BCR properties of DEs rules out cross contamination of DEs by B_{con} cells. In addition, the results indicate commonality between DEs at least in a subset of T1D patients represented by those analyzed in this study.

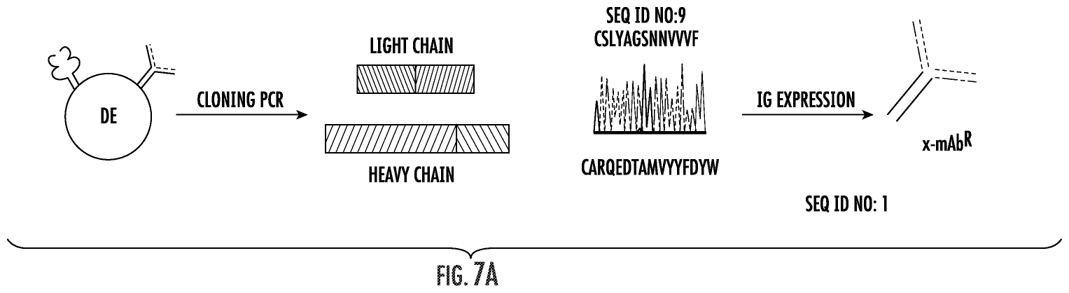

Intrigued by the predominant usage of the IGHV04-b by DEs, we analyzed them for clonality. Remarkably, IGHV04-b+ DEs were comprised of a single clonotype that used the same VH, DH and JH segments and N1 and N2 junctions in the three subjects, resulting in a CDR3 with identical nucleotide and amino acid sequences (FIG. 3E). The CDR3 (CARQEDTAMVYYFDYW) (SEQ ID NO:1) is encoded by rearranged IGHV04-b, IGHD05-18 and IGHJ04-01*02 (FIG. 3E and Table 5, not shown)—hereafter referred to as the x-clonotype. The dominance of the x-clonotype in the unrelated patients is unlikely coincidental given the extreme diversity of BCR repertoire (Truck et al., 2015; Venturi et al., 2008). Case in point, the clonal diversity of B_{con} cells in the three subjects (see Tables 5E, 5F and 5G, not shown). To confirm the identity of the x-clonotype, we sorted single DEs from T1D #1 and analyzed for IGHV expression using multiplex PCR (Smith et al., 2009). We detected the exact x-clonotype nucleotide sequence in 7 out of 7 DE cells (see FIGS. 6 and 7 and data not shown), confirming the high throughput sequencing results which identified the x-clonotype in 95% of DEs in T1D #1.

We also detected the x-clonotype among B_{con} cells in the three T1D subjects, but it was one of several small clonotypes that used the IGHV04-b gene (8 in T1D #1, 39 in T1D #2 and 17 in T1D #3) (Tables 5E, 5F and 5G, not shown). Furthermore, the identical amino acid sequence of the x-clonotype was generated by several B_{con} clonotypes that used multiple VH genes in T1D #1 (VH04-b; VH03-11; VH01-69; VH01-46; Vh05-51; VH01-18), T1D #2 (VH04-b; VH-04-39), and T1D #3 (VH04-b; VH03-53, VH01-02, VH1-69) patients (see Tables 5E, 5F and 5G, not shown). Generation of an identical CDR3 amino acid sequence by different VDJ rearrangements (convergent recombination) is a characteristic of public TCRs shared between at least two individuals (Venturi et al., 2008). In this regards, the x-clonotype was only one of two IGHV clonotypes (FIG. 3F)—(other less dominant one has a CAGGHNYGIKSYW (SEQ ID NO:20) CDR3 sequence) shared by B_{con} cells in the three T1D subjects. Thus, the x-clonotype predominated repertoires of DEs cells and was one of only two clonotypes shared between B_{con} cells of three T1D patients.

The x-clonotype is absent from repertoires of DEs of a healthy subject and public database. To shed further light into DEs and prevalence of the x-clonotype, we were able to obtain and analyze repertoires of IgD+ and IgD− DEs and compare to that of B_{con} cells in HC #1. We found that the repertoires of DEs in HC #1 were as diverse as that of B_{con} cells (FIG. 3G and Table 6, not shown). Usage of IGHV04-b gene was rare (<0.015%) in in IgD+, IgD− cells, and B_{con} cells of HC #1 (Table 6A, not shown). More importantly, the x-clonotype was absent from repertoires of IgD+, IgD− and B_{con} cells of HC #1 (Tables 6B, 6C and 6D, not shown). Nonetheless, IGHV04-b+ IgD+ cells in HC #1 as in T1D subjects were comprised of one clonotype that used the IGHJ04-01*2 gene, but not the DH05-18 gene, and their CDR3 sequence (CARQRFWSGPLFDYW) (SEQ ID NO:21) partly matched (boldfaced) that of x-clonotype. IGHV04-b+IgD− DE cells, however, were comprised of five clonotypes that used the IGHJ04-01*2, but not DH05-18 (FIG. 3H). Furthermore, DE clonotypes in HC #1, as in T1D patients, were of germline configuration albeit with few somatic mutations (FIG. 3I). Thus, repertoires of DEs in HC #1, unlike in the three T1D subjects, were diverse and did not include the x-clonotype.

Survey of public database showed that the x-clonotype was absent from high resolution immunoSEQ database (37 million unique BCR sequences) of naïve and memory B cells from healthy subjects (DeWitt et al., 2016) and that of insulin-binding B_{con} cells (IBCs) from T1D and control subjects (Seay et al., 2016)—potential reasons discussed below. Survey of NCBI protein database, however, discovered a highly overlapping sequence (RQENFDTAMVYYF) (SEQ ID NO:22) derived from the variant surface glycoprotein (VSG 1125.4290) of Trypanosoma brucei. Boldfaced letters indicate overlapping residues. VSGs are potent antigenic stimulators of B cells and Tindependent IgM response (Mansfield, 1994). We conclude that the x-clonotype is rarely used as indicated by its absence from the available database of IGHV sequences of B cells including IBCs.

X-clonotype is detectable in peripheral blood using sequence-specific PCR probes. We sought to detect the x-clonotype in peripheral blood using two sets of sequence-specific primers. In both cases, we used a forward primer specific for the VH04-b gene. In the first set (probe 1), it was paired with a reverse primer complementary to the entire CDR3 sequence (see diagram in FIG. 3J). Since our primary goal was to confirm the presence of the idiotype by an independent mechanism, we confined our analysis to a limited number of T1D and HC subjects (Tables 1B and 1C, not shown). We detected the x-clonotype in 4/8 T1D and 3/8 HCs. Detection of the x-clonotype in HCs prompted us to determine the HLA and islet autoantibody (IAA) profiles of participants. As expected all T1D subjects carried at least one risk allele (DQ2 or DQ8, hereafter referred to collectively as (357D-4) with one participant also expressed DQ7 (the disease-neutral isoform, DQB:3*01, of DQ8 that expresses D at β57). All T1D subjects were positive for at least one IAA and all HCs were negative for IAAs. Interestingly, the three x-clonotype+ HCs expressed DQ7, a DQ8 isoform that expresses D at β57. We did not detect the x-clonotype in the three HCs carrying at least one risk allele i.e. β57D-4, a result consistent with the absence of the x-clonotype from the high-throughput IGHV sequences of (357D-4 HC #1 described above (FIG. 3G). In the second probe (probe 2), to impart more stringent specificity, we used a reverse primer complementary to the N2-J and downstream JH sequence (see diagram, Table 7A, not shown). Again, we identified the x-clonotype in 4/9 T1D subjects, and 4/14 HCs (Table 7B and 7C, not shown). These subjects had not been genotyped or checked for IAAs. However, based on the results of the first probe, we speculate that x-clonotype+ HCs would include IAA−/DQ7+ and at risk IAA+/β57D−/+ individuals.

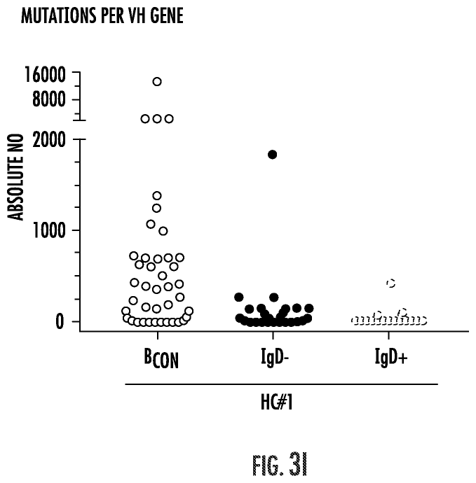

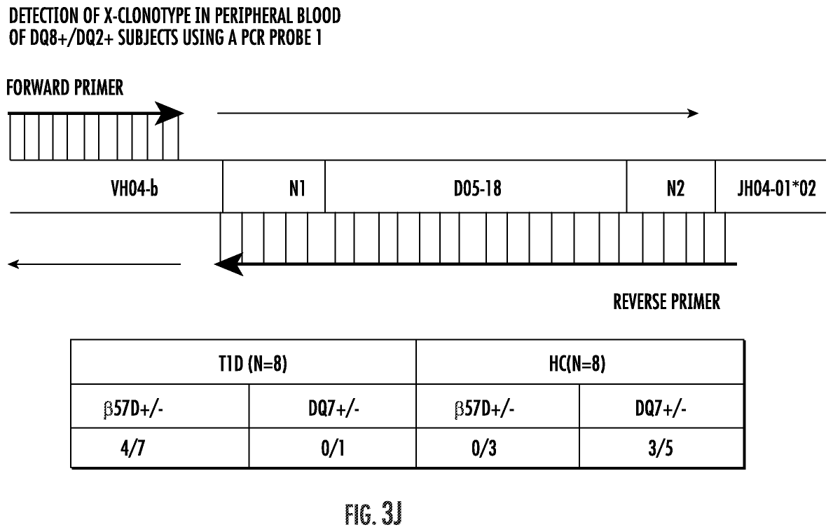

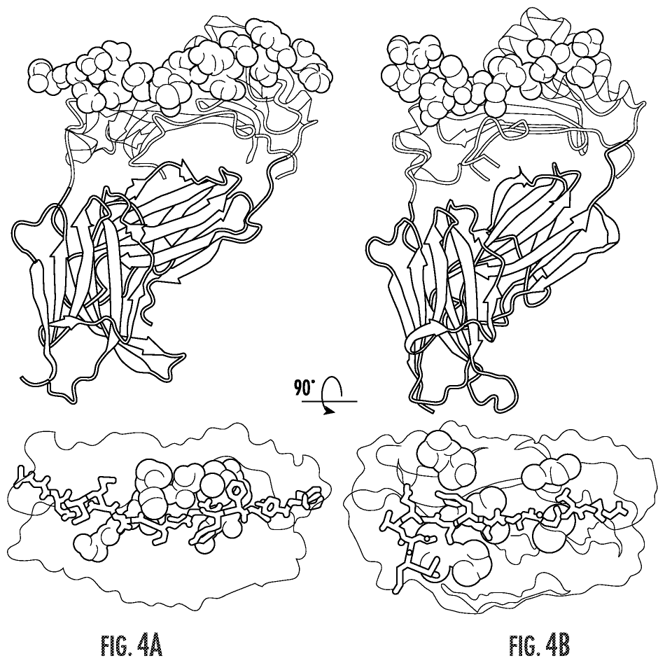

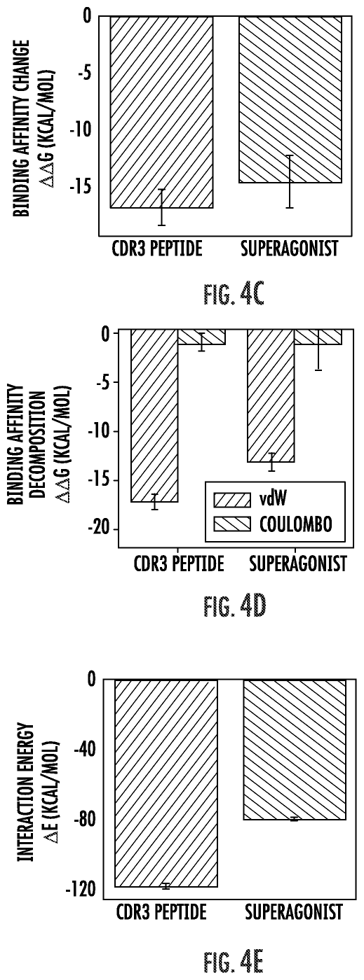

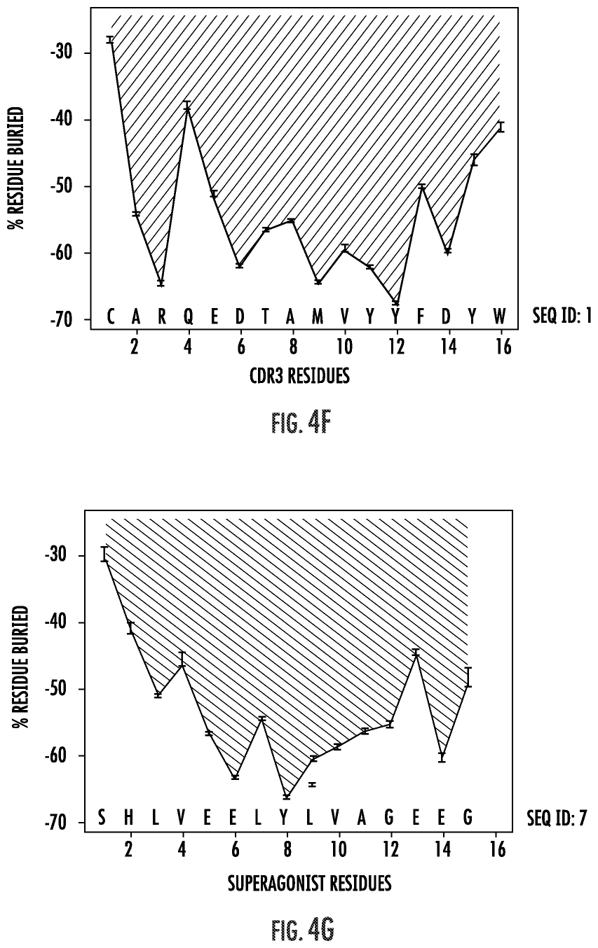

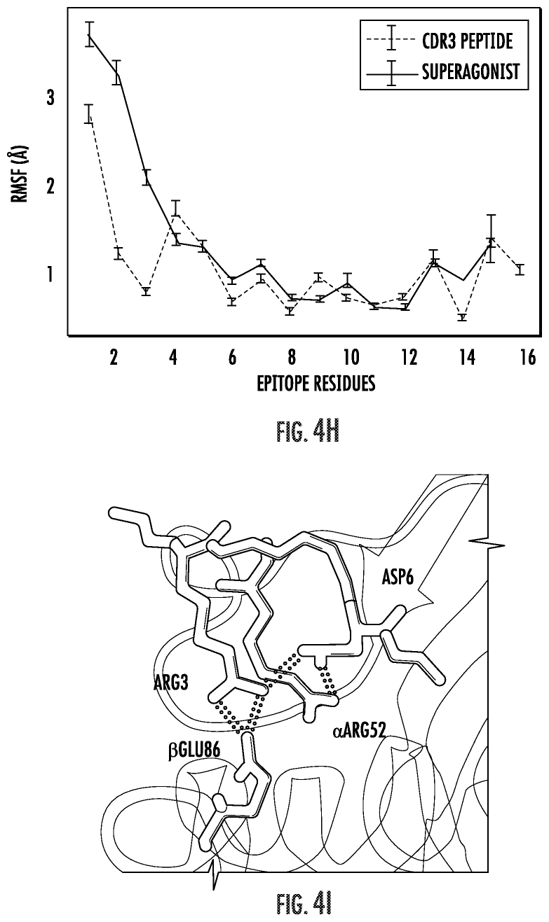

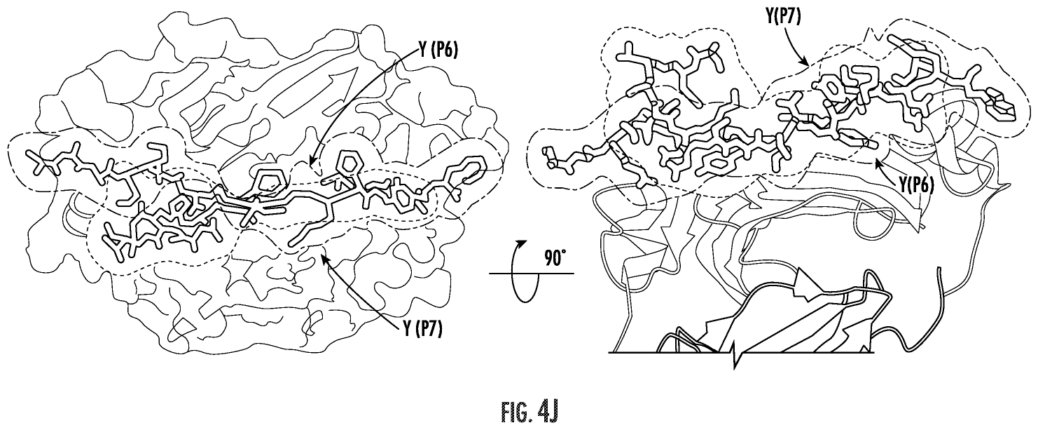

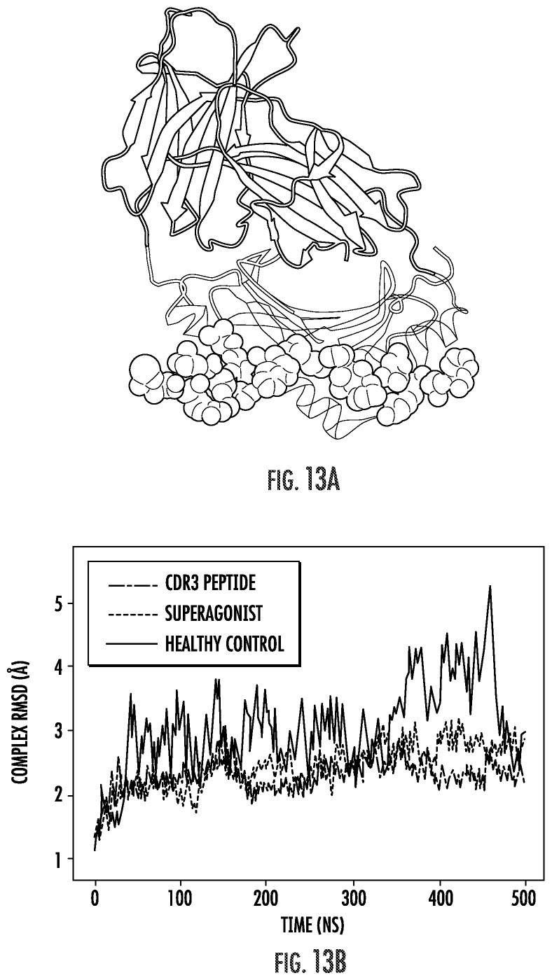

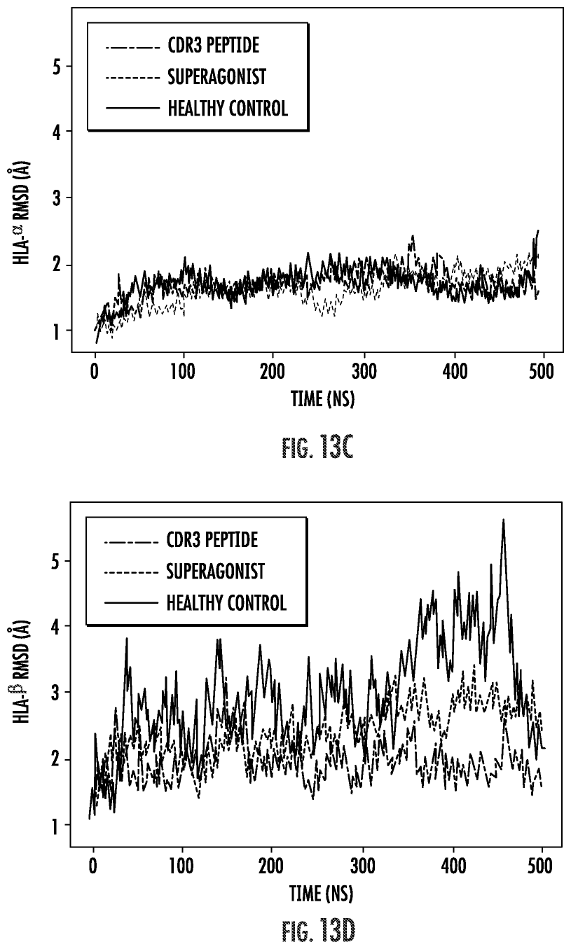

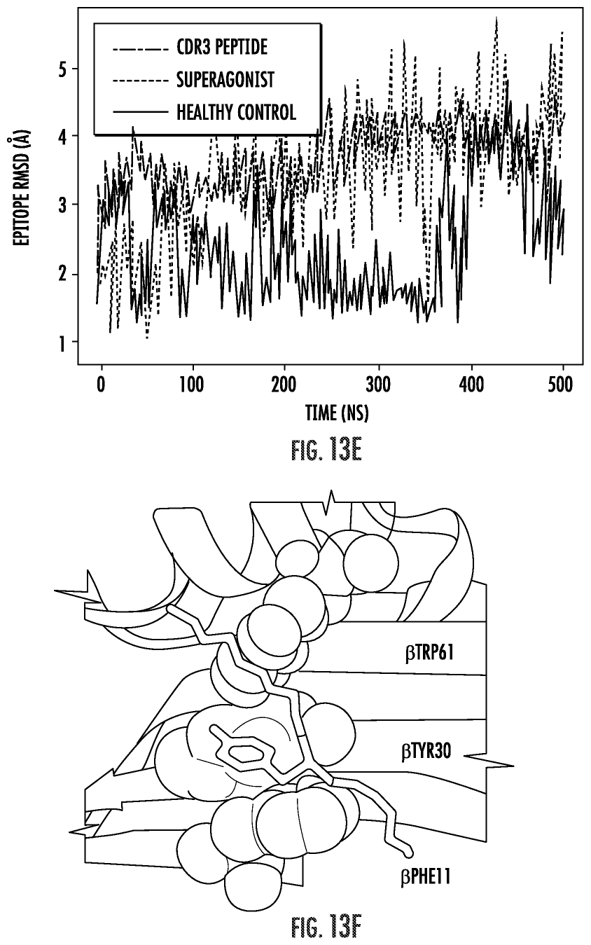

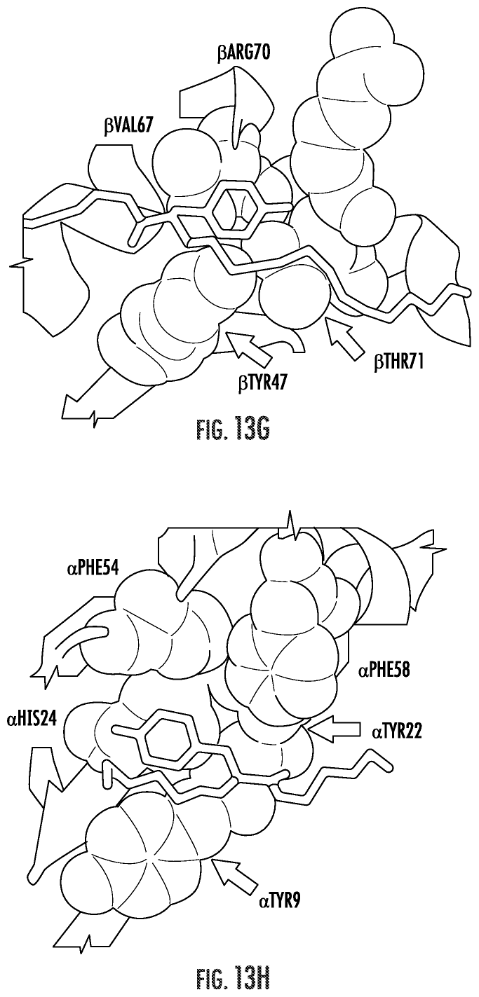

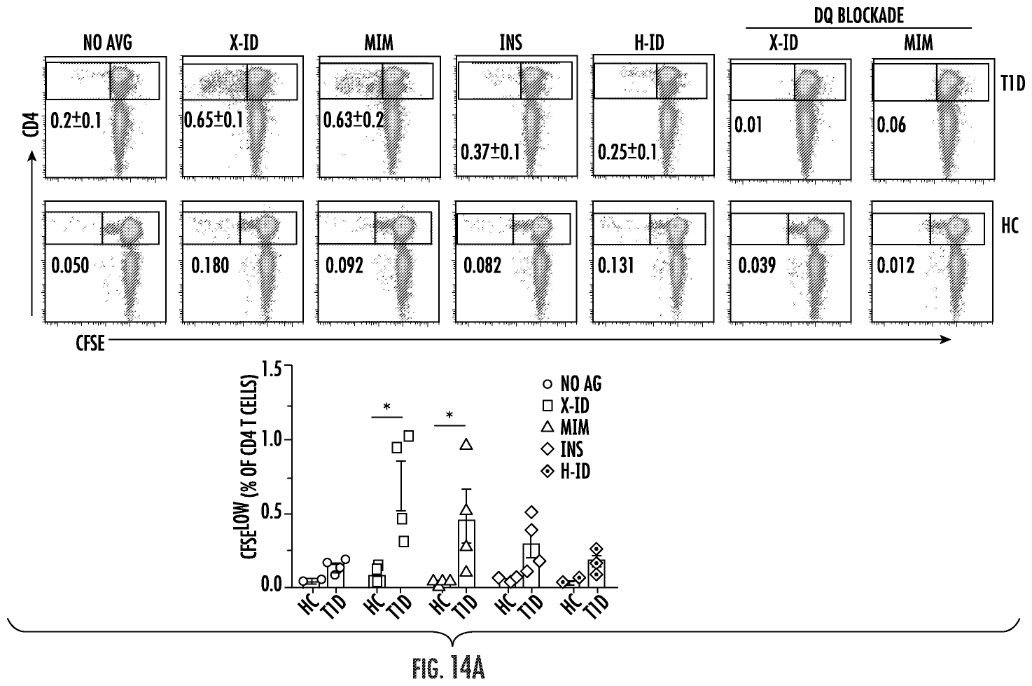

Molecular Dynamics simulations (MDS) identify the x-clonotype as an optimal peptidome for DQ8. As mentioned above, combining R22E at P9 and A14E at P1 substitutions generates an insulin superagonist with high affinity for DQ8 as shown in a recently published crystal structure (Wang et al., 2018). Alignment analysis predicted that the x-clonotype could include a DQ8 binding epitope with acidic residues (E or D) at the P1 and P9 positions, similar to that of the superagonist. To test this prediction and further characterize CDR3 peptide-HLA loading, we conducted computational biophysical modeling of epitope-HLA binding, which has been successfully used in several previous studies (Chowell et al., 2018; Holzemer et al., 2015; Joglekar et al., 2018; Xia et al., 2014). The binding complexes of HLA with the CDR3 peptide (CARQEDTAMVYYFDYW (SEQ ID NO:1), core epitope underlined) and superagonist (SHLVEELYLVAGEEG) (SEQ ID NO:7), as well as their associated binding energies, are shown in (FIG. 4). We first ran Molecular Dynamics (MD) simulations of three HLA-epitope complexes (CDR3, superagonist, healthy control CARQRFWSGPLFDYW) (SEQ ID NO:21) to assess stability of the bound epitopes (for Methods, see supplementary material). We found that all three epitopes remained bound to the HLA without register shifts from their initial anchoring sites; however, the HC epitope destabilized the HLA-τ3 subunit (backbone RMSD>4 Å, FIG. 13) indicating unfavorable binding. Hence, subsequent analyses were limited to the CDR3 peptide and superagonist. The final bound structures are shown in (FIG. 4A) for the CDR3 peptide (side and top views, respectively) and (FIG. 4B) for the superagonist, with both displaying strong binding to DQ8. Free Energy Perturbation (FEP) calculations reveal that the CDR3 peptide binds even stronger than the superagonist (FIGS. 4C and 4D). In general, the FEP method can compute the binding affinity difference of alchemically mutating from one epitope to another (Chowell et al., 2018; Holzemer et al., 2015; Joglekar et al., 2018; Xia et al., 2014) (see Methods). However, given that the epitope sequences diverge greatly in the current case with only one conserved residue (Val10), we computed the binding affinity change (AG) for the mutation of each epitope to a neutral polyglycine (peptide backbone) intermediate, which serves as a reference point. The relative binding affinity between different epitopes can be easily calculated from the relative differences of the values of ΔG for each epitope. Our results show that the CDR3 peptide has more favorable binding affinity than the superagonist by −2.3±2.8 kcal/mol (FIG. 4C). Decomposition of the binding affinity reveals a −4 kcal/mol van der Waals interaction preference for CDR3 binding over the superagonist (FIG. 4D). This is supported by a simple interaction energy comparison as shown in FIG. 4E where the CDR3 peptide displays a stronger van der Waals interaction than the superagonist. Overall, these binding affinity results agree well with the above in vitro experimental binding assays where the CDR3 peptide was found to be a more potent autoantigen.

Further in-depth structural analyses reveal several beneficial binding characteristics of this super potent CDR3 peptide (FIGS. 4F, 4G, 4H, 4I and 4J). The importance of the anchor residues at sites 1, 4, 6 and 9 of the CDR3 core epitope is clearly visible from contact analyses (FIGS. 4F and 4G). Interestingly, the tyrosine residues of CDR3 site 7 and the superagonist site 3 hold the largest normalized contact area, making extensive contacts with aromatic and hydrophobic HLA residues (see FIGS. 13G and 13H). Residue fluctuations, as presented in (FIG. 4H), often hint at which residues are rigorously bound to the HLA. Despite not being in the ‘core epitope’, the N-terminal CDR3 residues bound favorably to the HLA once the Arg3 and Asp6 formed a robust yet intricate buried salt bridge complex with β86E and α52R of the HLA (FIG. 4I). In contrast, the N-terminal superagonist residues SHL not only had lower contact numbers with the HLA (FIG. 4G), but also displayed larger fluctuations throughout the simulation (FIGS. 4H, 4I and 4J). Furthermore, in addition to the tyrosine residue of CDR3 at position 7 [Y (P7)] as mentioned above (making strong π-π stacking with β47Y and hydrophobic interactions with β67V, (FIG. 13G), the core tyrosine residue at position 6 [Y (P6)] also contributed favorably to binding by forming favorable π-π interactions with β11F, β30Y, and β61W (FIGS. 4J and 13F). Taken together, we conclude the strong π/π (stacking and hydrophobic interactions from CDR3 peptide's aromatic residues Y(P6) and Y(P7) contributed favorably to stronger binding with HLA, while the large fluctuation of the superagonist N-terminus contributed slightly unfavorably towards its lower binding affinity. Taken together, results of MDS show that the CDR3 peptide (x-Id) peptide appears to have optimal anchor residues for binding to DQ8.

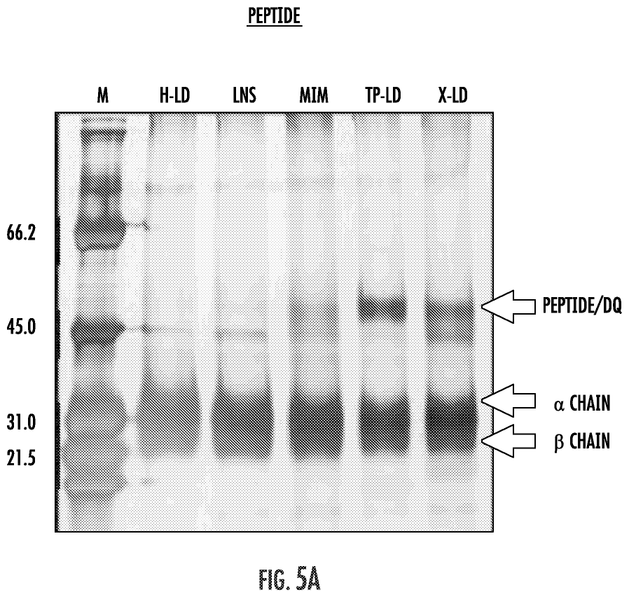

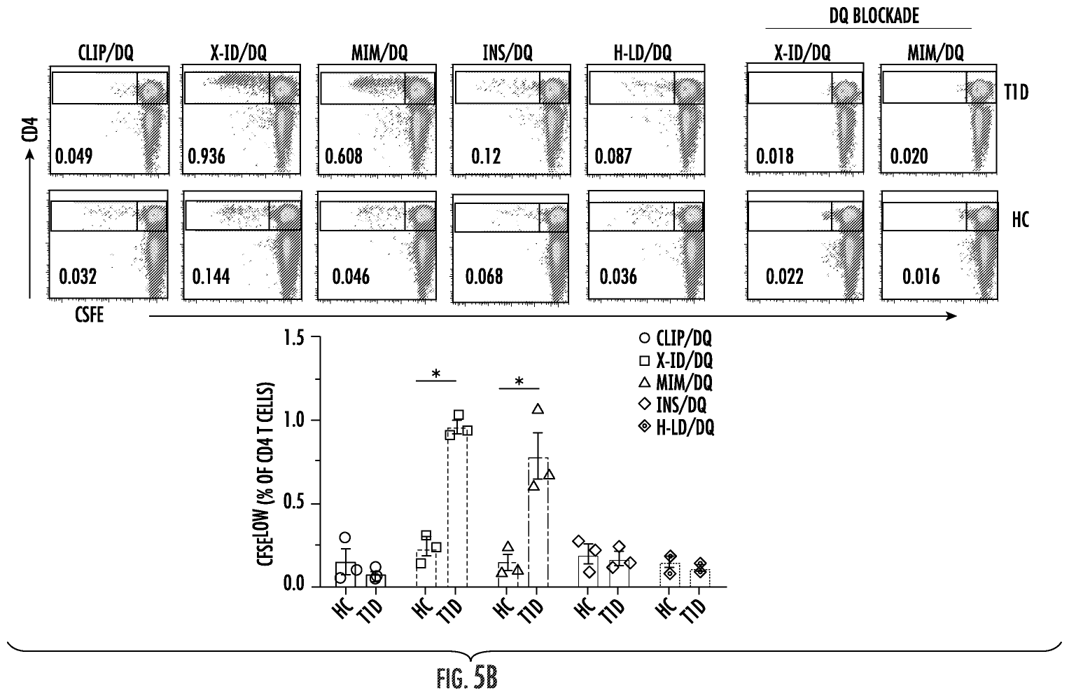

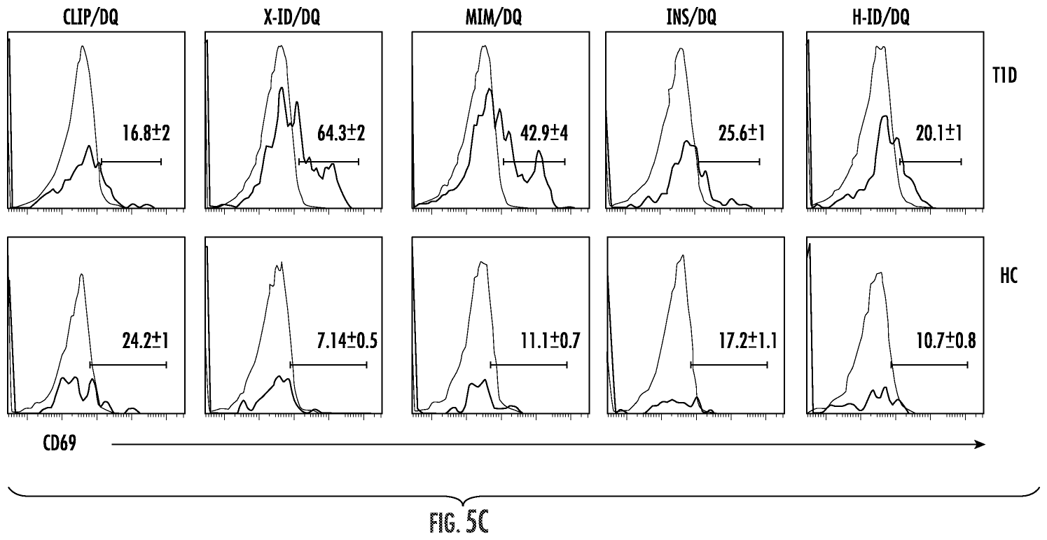

The CDR3 sequence of x-clonotype is a potent CD4 T cell epitope. Expansion of the x-clonotype-expressing DEs in T1D and the unique DQ8 binding properties of the x-Id peptide suggest a connection to the disease pathogenesis. We considered and excluded the possibility that the x-clonotype encodes an IAA because IAAs generally use VH06, have net positive charges, and long CDR3 (Smith et al., 2015). In contrast, the x-clonotype uses VH04, has a net negative charge (−2.01) and normal CDR3 length. Moreover, as mentioned above, the x-clonotype is absent from published sequences of IBCs. An alternative hypothesis is that the x-clonotype encodes a previously unknown DQ8-restricted CD4 T cell neoantigen. This hypothesis is directly supported by the results of MDS analysis (see FIG. 4). To functionally test this idea, we examined the ability of two idiotypic peptides to form stable DQ8 complexes using a gentle SDS-PAGE assay (Kim et al., 2013; Sadegh-Nasseri and Germain, 1991). One peptide is the full CDR3 sequence, CARQEDTAMVYYFDYW (x-Id) (SEQ ID NO:1), and the second is a truncated version (TP-Id) that lacked cysteine (C) at the C terminal and tryptophan (W) at the N terminal—CARQEDTAMVYYFDYW (SEQ ID NO:1). We used native insulin B: 9-23 and the mimotope as controls. We also tested binding of the CDR3 peptide (CARQRFWSGPLFDYW) (SEQ ID NO:1) of the IGVH04-b+ IgD+ clonotype from HC #1. Both idiotypic peptides (x-Id and TP-Id) were able to bind soluble DQ8 molecules forming SDS-stable complexes as did the mimotope (FIG. 5A). In contrast, the HC (h-Id) and native insulin peptides did not form detectable DQ8 complexes. These results show that x-idiotype (x-Id) from T1D, but not HC (h-Id), can form SDS-stable complexes with DQ8. Using CFSE proliferation assay and CD69 upregulation, we demonstrated that x-Id/DQ8 complexes, similar to mim/DQ8 complexes, were potent stimulators of CD4 T cells from DQ8+ T1D subjects. Responders included T1D #1 in whom most of DEs expressed the IGHV04-b+ clonotype, hence an autoreactive response. Consistent with their poor binding to DQ8, native insulin and HC (h-Id) peptides generated no significant responses. The proliferative responses, as expected, were inhibited by anti-DQ8 mAb (FIG. 5B). Importantly, x-Id/DQ8 complexes induced weak or no responses from healthy subjects, indicating their high reactivity is associated with T1D (FIG. 5B). In addition, most of CFSElow CD4 T cells upregulated CD69 as compared to their CFSEhi counterparts (FIG. 5C). Similar MHC class II-dependent responses results were obtained when the x-Id peptide was used to pulse PBMCs (FIG. 14). These results identify for the first time a potent T cell autoantigen that is encoded in the idiotype of a public BCR.

Verification of dual expression of BCR and TCR by DEs using an EBV-immortalized clone. Next, we successfully generated an EBV-immortalized lymphoblastoid cell line (x-LCL) from sorted DEs that were isolated from T1D #1 and transformed using an established method (Caputo and Flytzanis, 1991; Hui-Yuen et al., 2011). Given that almost all DEs in T1D #1 expressed the x-clonotype (see FIG. 3), we used x-LCL cells to clone and characterize the antibody encoding the x-clonotype. We sorted single cells from cultured x-LCL and examined each for transcripts of heavy and light chains. We confirmed expression of the x-clonotype in 7/7 sorted single x-LCL cells using the same primers described above for detection of the x-clonotype in fresh DE cells (FIG. 6A). Analysis of transcripts of the light chain identified two light chains: a dominant productive light chain (IGL1-x with CDR3: CSLYAGSNNVVVF (SEQ ID NO:9)) and a minor non-productive chain 2 (IGL2-x with CDR3: VVYMQAATMLWYS (SEQ ID NO:8)) that was detected in two cells that could possibly express productive light chain(s) that were not picked by the PCR. Alternatively, IGL1-a and IGL2-x may be representing the same light chains because the IGL2-x has the same nucleotide sequence of the IGL1-x except for missing three nts, which could be caused by PCR or reading errors. Thus, the antibodies expressed in DEs, at least in T1D #1, used the x-clonotype paired with at least one productive light chain.

Subsequently, we took advantage of x-LCL cells to verify coexpression of BCR and TCR in DE cells. We used limiting dilution (0.3 cell/well) and generated one clone (referred to as x1.1) that was confirmed by cloning PCR to express the IGL1 light chain paired with the x-clonotype (FIG. 6A). In addition, we have visualized coexpression of TCR, IgD, and IgM at the single cell level using florescence imaging. Finally, using RT-PCR and nested PCR, we successfully amplified and cloned a TCRαβ composed of TRBV6-5*01/D 1*01, JB1-1*01 (TCRβ-x) and TRAV29/DV5*01/J53*01 (TCRα-x) from cells of the x1.1 clone (FIG. 6A). Detection of fully rearranged and expressed BCR and TCR chains from cells of the x1.1 clone confirms their dual expression in DEs.

x-Id-peptide and insulin mimotope recognize overlapping subpopulations of CD4 T cells. Antibodies can activate T cells by being sources of soluble autoantigens (Khodadoust et al., 2017) and idiotypic-specific CD4 T cells have been described in multiple sclerosis (Hestvik et al., 2007) and lupus (Aas-Hanssen et al., 2014). As shown above, the x-Id peptide can serve as an autoantigen by forming functional complexes with DQ8 molecules (see FIG. 5). Cultured x1.1 cells secreted copious amounts of an x-clonotype-encoding antibody, herein referred to as x-mAbN. We assessed the ability of the x-mAbN to stimulate CD4 T cells from T1D subjects using the CFSE proliferation assay (FIG. 6B). Addition of soluble x-mAbN to PBMCs resulted in potent proliferation as indicated by the percentage of CFSElow CD4 T cells. We have also generated a recombinant x-mAb of IgG1 isotype (referred to x-mAbR). We cloned the variable regions of the heavy and light chains from sorted single DE cell isolated from T1D #1 and fused into human IgG vectors as described in methods (FIG. 7A). Similar to its natural counterpart, immobilized x-mAbR activated CD4 T cells in a CFSE proliferation assay (FIG. 7B). Thus, both naturally-produced and recombinant x-mAbs are stimulatory for CD4 T cells.

To examine the relationship between x-Id- and insulin-reactive CD4 T cells, we generated DQ8 tetramers that were loaded with x-Id peptide (x-tetramer) or mimotope peptide (mim-tetramer). We used CLIP-DQ8 tetramer (CLIP-tetramer) as a specificity. We cultured PBMCs from T1D subjects in the presence or absence of x-Id peptide or insulin mimoptope for 7 days and analyzed each culture for the presence of tetramer-positive CD4 T cells (FIG. 7C). We detected x-Id and mimotope specific CD4 T cells in both unstimulated and stimulated cultures, using CLIP-tet staining as a negative control. Furthermore, percentages of tetramer positive cells were significantly higher in stimulated cultures as compared to unstimulated cultures. More importantly, the x-tetramer was able to detect CD4 T cells expanded by the insulin mimotope and the opposite was true for the mim-tetramer which detected CD4 T cells expanded by the x-Id peptide. These results suggested the mimotope and x-Id peptide stimulate the same or overlapping subpopulations of CD4 T cells. In direct support of this notion, x-mAb significantly inhibited binding of the mim-tetramer to activated CD4 T cells (FIG. 7C). Thus, it appears that x-clonotype cross-activates insulin-specific CD4 T cells.

### Discussion

This study describes rare lymphocytes (DEs) that clonally expand in T1D subjects and bore lineage markers of both B and T cells epitomized by expression of TCR and BCR. Clonally expanded DEs encode a potent autoantigen (x-autoantigen) in the antigen binding site of the Ig heavy chain with an optimal register for diabetogenic DQ8 molecules. The x-autoantigen peptide forms functional complexes with DQ8 molecules that robustly stimulate CD4 T cells from T1D, but not HC subjects. In addition, x-clonotype-bearing mAbs also stimulate CD4 T cells. Competitive binding inhibition analysis indicates that the x-mAb and insulin mimotope stimulate overlapping T cell subpopulations (FIG. 7C). Taken together these findings indicate that compartmentalization of T and B cells is not absolute and violators of this paradigm could be key drivers of autoimmunity.

Features of the x-autoantigen and implications for T1D. Molecular dynamic simulations show that the x-Id peptide has an optimal binding register for DQ8 with negatively charged residues at P1 and P9. Significance of having acidic residues at these critical anchor positions is indicated by the transformation of native insulin B:9-23 peptide into a potent superagonist with strong binding ability to DQ8 molecules by substituting glutamic acids for alanine at P1 and arginine at P9. In concordance, the x-Id peptide and insulin mimotope have comparable class II-restricted T cell stimulatory abilities. Given that BCR idiotypes are frequently processed and presented in the context of MHCII as neoantigens to CD4 T cells (Khodadoust et al., 2017), it is conceivable that DEs serve as a source of x-Id epitopes in vivo. In support of this possibility, idiotypic-specific CD4 T cells have been described in multiple sclerosis (Hestvik et al., 2007) and lupus (Aas-Hanssen et al., 2014). To our knowledge, the cellular sources of idiotypic autoantigens in these diseases have not been identified, however. Therefore, it will be important to analyze patients with different autoimmune diseases for the presence, clonal expansion and specificities of DEs. It will also be important to search peptide repertoires eluted from MHC class II molecules from T1D subjects for the presence of x-Id peptide to determine whether our in vitro findings could be linked to the disease pathogenesis. Presence of x-Id/DQ8 complexes in vivo is possible given the clonal expansion of DEs bearing the x-Id autoantigen in T1D subjects and convergent recombination in B_{con} cells as we detected as many as 10 B_{con} clonotypes in the immunoSEQ repertoires of B_{con} cells in the three T1D subjects that used different VH genes joined to the D 5-18/JH04-2 gene segments to generate CDR3s that encode the amino acid sequence of the x-autoantigen albeit present at very low frequencies. Alternatively, but not mutually exclusive, DEs can influence pathogenesis of T1D by secretion of mAbs encoding the x-autoantigen in their heavy chain idiotypes. Our results show that antibodies (x-mAbN) secreted by immortalized DEs or cloned from fresh DE cell (x-mAbR) are potent stimulators of CD4 T cells. In addition, x-mAbR significantly inhibited binding of the mimotope tetramer to autoreactive CD4 T cells, suggesting overlapping specificities. Examination of sera from T1D subjects for the x-mAb in the future would provide new insights into pathophysiologic roles in disease pathogenesis.

Expression of functional TCR on DE cells and pathogenic implications. Expression of the TCR on DEs could have important implications. For one, it gives DEs the ability to expand and increase their numbers upon TCR stimulation. In addition, crosslinking of TCR on DEs leads to upregulation of MHCII and costimulatory molecules, including CD40 and CD80/CD86 thereby converting DEs into potentially potent APCs. These features will be pathogenically relevant if future analysis show that DEs process and present x-Id peptide on their surface DQ molecules and/or use membrane Ig to stimulate autoreactive CD4 T cells. TCR stimulation of DEs also leads to production of cytokines that can influence their local milieus. Thus, future studies should investigate antigen specificity of TCRs expressed on DEs including reactivity against environmental antigens and common pathogens.

Immediate translational impacts and future significance. Our results being derived from studying peripheral blood samples from T1D and HCs are expected to have translational significance. One immediately testable question is whether presence of the x-autoantigen in peripheral blood serves as a biomarker that distinguish between at-risk individuals. In the numbers of subjects examined in this study, the x-clonotype was detected in peripheral blood of T1D and also in HC expressing DQ7 (the neutral isoform of DQ8 that express Aspartic acid at position 57 of the β chain), but not in HC-bearing risk (β57D−) DQ alleles. Thus, it is possible that β57D− subjects who are negative for the x-clonotype are at low risk for developing T1D. On the other hand, the DQ7 molecule, by virtue of having Aspartic acid at β57 position, will favor a positively charged residue at P9 and hence cannot optimally bind the x-clonotype at the least in the same register as the DQ8 molecule does. This difference could explain the neutral role of DQ7 as a risk factor for T1D.

The limited numbers of single DE cells analyzed and short read length (38 bp) prevented us from determining whether DEs represent a distinct or a subpopulation of an existing cell type and from accurately analyzing their CDR3 sequences in scRNA-seq settings (Rizzetto et al., 2017). These are goals of our future experiments. Furthermore, if the x-clonotype proved to be a key driver of T1D, its conserved germline sequence will be useful in developing antigen-specific therapeutic strategies. Furthermore, the unique surface phenotype of DEs make them an easily detectable target and because of their small numbers their elimination might not have significant negative effect on host defense.

### REFERENCES

1. Aas-Hanssen, K., Funderud, A., Thompson, K. M., Bogen, B., and Munthe, L. A. (2014). Idiotype-specific Th cells support oligoclonal expansion of anti-dsDNA B cells in mice with lupus. Journal of immunology 193, 2691-2698.
2. Best, R. B., Zhu, X., Shim, J., Lopes, P. E., Mittal, J., Feig, M., and Mackerell, A. D., Jr. (2012). Optimization of the additive CHARMM all-atom protein force field targeting improved sampling of the backbone phi, psi and side-chain chi(1) and chi(2) dihedral angles. J Chem Theory Comput 8, 3257-3273.
3. Canzar, S., Neu, K. E., Tang, Q., Wilson, P. C., and Khan, A. A. (2017). BASIC: BCR assembly from single cells. Bioinformatics 33, 425-427.
4. Caputo, J. G., and Flytzanis, N. (1991). Kink-antikink collisions in sine-Gordon and phi4 models: Problems in the variational approach. Phys Rev A 44, 6219-6225.
5. Carlson, C. S., Emerson, R. O., Sherwood, A. M., Desmarais, C., Chung, M. W., Parsons, J. M., Steen, M. S., LaMadrid-Herrmannsfeldt, M. A., Williamson, D. W., Livingston, R. J., et al. (2013). Using synthetic templates to design an unbiased multiplex PCR assay. Nat Commun 4, 2680.
6. Chang, K N., and Unanue, E. R. (2009). Prediction of HLA-DQ8beta cell peptidome using a computational program and its relationship to autoreactive T cells. International immunology 21, 705-713.
7. Chowell, D., Morris, L. G. T., Grigg, C. M., Weber, J. K., Samstein, R. M., Makarov, V., Kuo, F., Kendall, S. M., Requena, D., Riaz, N., et al. (2018). Patient HLA class I genotype influences cancer response to checkpoint blockade immunotherapy. Science 359, 582-587.
8. Crawford, F., Stadinski, B., Jin, N., Michels, A., Nakayama, M., Pratt, P., Marrack, P., Eisenbarth, G., and Kappler, J. W. (2011). Specificity and detection of insulin-reactive CD4+ T cells in type 1 diabetes in the nonobese diabetic (NOD) mouse. Proceedings of the National Academy of Sciences of the United States of America 108, 16729-16734.
9. Dai, H., Rahman, A., Saxena, A., Jaiswal, A. K., Mohamood, A., Ramirez, L., Noel, S., Rabb, H., Jie, C., and Hamad, A. R. (2015). Syndecan-1 identifies and controls the frequency of IL-17-producing naive natural killer T (NKT17) cells in mice. European journal of immunology.
10. DeWitt, W. S., Lindau, P., Snyder, T. M., Sherwood, A M., Vignali, M., Carlson, C. S., Greenberg, P. D., Duerkopp, N., Emerson, R. O., and Robins, H. S. (2016). A Public Database of Memory and Naive B-Cell Receptor Sequences. PloS one 11, e0160853.
11. Eugster, A., Lindner, A., Heninger, A. K., Wilhelm, C., Dietz, S., Catani, M., Ziegler, A. G., and Bonifacio, E. (2013). Measuring T cell receptor and T cell gene expression diversity in antigen-responsive human CD4+ T cells. J Immunol Methods 400-401, 13-22.
12. Hamad, A. R., Herman, A., Marrack, P., and Kappler, J. W. (1994). Monoclonal antibodies defining functional sites on the toxin superantigen staphylococcal enterotoxin B. The Journal of experimental medicine 180, 615-621.
13. Hestvik, A. L., Vartdal, F., Fredriksen, A. B., Thompson, K. M., Kvale, E. O., Skorstad, G., Bogen, B., and Holmoy, T. (2007). T cells from multiple sclerosis patients recognize multiple epitopes on Self-IgG. Scandinavian journal of immunology 66, 393-401.
14. Holm, S. (1979). Simple Sequentially rejective multiple test procedure. 6.
15. Holzemer, A., Thobakgale, C. F., Jimenez Cruz, C. A., Garcia-Beltran, W. F., Carlson, J. M., van Teijlingen, N. H., Mann, J. K., Jaggernath, M., Kang, S. G., Korner, C., et al. (2015). Selection of an HLA-C*03:04-Restricted HIV-1 p24 Gag Sequence Variant Is Associated with Viral Escape from KIR2DL3+ Natural Killer Cells: Data from an Observational Cohort in South Africa. PLoS medicine 12, e1001900; discussion e1001900.
16. Hui-Yuen, J., McAllister, S., Koganti, S., Hill, E., and Bhaduri-McIntosh, S. (2011). Establishment of Epstein-Barr virus growth transformed lymphoblastoid cell lines.

Journal of visualized experiments: JoVE.
17. Joglekar, A. V., Liu, Z., Weber, J. K., Ouyang, Y., Jeppson, J. D., Noh, W. J., Lamothe-Molina, P. A., Chen, H., Kang, S. G., Bethune, M. T., et al. (2018). T cell receptors for the HIV KK10 epitope from patients with differential immunologic control are functionally indistinguishable. Proceedings of the National Academy of Sciences of the United States of America 115, 1877-1882.
18. Khodadoust, M. S., Olsson, N., Wagar, L. E., Haabeth, O. A., Chen, B., Swaminathan, K., Rawson, K., Liu, C. L., Steiner, D., Lund, P., et al. (2017). Antigen presentation profiling reveals recognition of lymphoma immunoglobulin neoantigens. Nature 543, 723-727.
19. Kim, A., Ishizuka, I., Hartman, I., Poluektov, Y., Narayan, K., and Sadegh-Nasseri, S. (2013). Studying MHC class II peptide loading and editing in vitro. Methods in molecular biology 960, 447-459.
20. LeMaoult, J., Caumartin, J., and Carosella, E. D. (2007). Exchanges of membrane patches (trogocytosis) split theoretical and actual functions of immune cells. Human immunology 68, 240-243.
21. Luisiri, P., Lee, Y. J., Eisfelder, B. J., and Clark, M. R. (1996). Cooperativity and segregation of function within the Ig-alpha/beta heterodimer of the B cell antigen receptor complex. J Biol Chem 271, 5158-5163.
22. Lun, A. T., McCarthy, D. J., and Marioni, J. C. (2016). A step-by-step workflow for low-level analysis of single-cell RNA-seq data with Bioconductor. F1000Res 5, 2122.
23. Mansfield, J. M. (1994). T cell responses to the trypanosome variant surface glycoprotein: a new paradigm? Parasitology today 10, 267-270.
24. Martina, M. N., Noel, S., Saxena, A., Bandapalle, S., Majithia, R., Jie, C., Arend, L. J., Allaf, M. E., Rabb, H., and Hamad, A. R. (2015). Double-Negative alphabeta T

Cells Are Early Responders to AM and Are Found in Human Kidney. J Am Soc Nephrol.
25. Michel, M. L., Pang, D. J., Hague, S. F., Potocnik, A. J., Pennington, D. J., and Hayday, A. C. (2012). Interleukin 7 (IL-7) selectively promotes mouse and human IL-17-producing gammadelta cells. Proceedings of the National Academy of Sciences of the United States of America 109, 17549-17554.
26. Nakayama, M., McDaniel, K., Fitzgerald-Miller, L., Kiekhaefer, C., Snell-Bergeon, J. K., Davidson, H. W., Rewers, M., Yu, L., Gottlieb, P., Kappler, J. W., et al. (2015). Regulatory vs. inflammatory cytokine T cell responses to mutated insulin peptides in healthy and type 1 diabetic subjects. Proceedings of the National Academy of Sciences of the United States of America 112, 4429-4434.
27. Okada, T., Miller, M. J., Parker, I., Krummel, M. F., Neighbors, M., Hartley, S. B., O'Garra, A., Cahalan, M. D., and Cyster, J. G. (2005). Antigen-engaged B cells undergo chemotaxis toward the T zone and form motile conjugates with helper T cells. PLoS biology 3, e150.
28. Pavlidis, P., and Noble, W. S. (2001). Analysis of strain and regional variation in gene expression in mouse brain. Genome biology 2, RESEARCH0042.
29. Picelli, S., Faridani, O. R., Bjorklund, A. K., Winberg, G., Sagasser, S., and Sandberg, R. (2014). Full-length RNA-seq from single cells using Smart-seq2. Nature protocols 9, 171-181.
30. Reth, M. (1992). Antigen receptors on B lymphocytes. Annual review of immunology 10, 97-121.
31. Rizzetto, S., Eltahla, A. A., Lin, P., Bull, R., Lloyd, A. R., Ho, J. W. K., Venturi, V., and Luciani, F. (2017). Impact of sequencing depth and read length on single cell RNA sequencing data of T cells. Scientific reports 7, 12781.
32. Robins, H. S., Campregher, P. V., Srivastava, S. K., Wacher, A., Turtle, C. J., Kahsai, O., Riddell, S. R., Warren, E. H., and Carlson, C. S. (2009). Comprehensive assessment of T cell receptor beta-chain diversity in alphabeta T cells. Blood 114, 4099-4107.
33. Sadegh-Nasseri, S., and Germain, R. N. (1991). A role for peptide in determining MHC class II structure. Nature 353, 167-170.
34. Saxena, A., Yagita, H., Donner, T. W., and Hamad, A. R. A. (2017). Expansion of FasL-Expressing CD5+ B Cells in Type 1 Diabetes Patients. Frontiers in immunology 8, 402.
35. Seay, H. R., Yusko, E., Rothweiler, S. J., Zhang, L., Posgai, A. L., Campbell-Thompson, M., Vignali, M., Emerson, R. O., Kaddis, J. S., Ko, D., et al. (2016). Tissue distribution and clonal diversity of the T and B cell repertoire in type 1 diabetes. JCI Insight 1, e88242.
36. Sewell, A. K. (2012). Why must T cells be cross-reactive? Nature reviews Immunology 12, 669-677.
37. Sharp, K. A. (2012). Statistical thermodynamics of binding and molecular recognition models (Wiley-VCH Verlag GmbH & Co. KGaA.).
38. Smith, K., Garman, L., Wrammert, J., Zheng, N. Y., Capra, J. D., Ahmed, R., and Wilson, P. C. (2009). Rapid generation of fully human monoclonal antibodies specific to a vaccinating antigen. Nature protocols 4, 372-384.
39. Smith, M. J., Packard, T. A., O'Neill, S. K., Henry Dunand, C. J., Huang, M., Fitzgerald-Miller, L., Stowell, D., Hinman, R. M., Wilson, P. C., Gottlieb, P. A., et al. (2015). Loss of anergic B cells in prediabetic and new-onset type 1 diabetic patients. Diabetes 64, 1703-1712.
40. Tirosh, I., and Suva, M. L. (2018). Dissecting human gliomas by single-cell RNA sequencing. Neuro-oncology 20, 37-43.
41. Tirosh, I., Venteicher, A S., Hebert, C., Escalante, L. E., Patel, A. P., Yizhak, K., Fisher, J. M., Rodman, C., Mount, C., Filbin, M. G., et al. (2016). Single-cell RNA-seq supports a developmental hierarchy in human oligodendroglioma. Nature 539, 309-313.
42. Truck, J., Ramasamy, M. N., Galson, J. D., Rance, R., Parkhill, J., Lunter, G., Pollard, A. J., and Kelly, D. F. (2015). Identification of antigen-specific B cell receptor sequences using public repertoire analysis. Journal of immunology 194, 252-261.
43. Venturi, V., Price, D. A., Douek, D. C., and Davenport, M. P. (2008). The molecular basis for public T cell responses? Nature reviews Immunology 8, 231-238.
44. Wang, J., Sohn, H., Sun, G., Milner, J. D., and Pierce, S. K. (2014). The autoinhibitory C-terminal SH2 domain of phospholipase Cgamma2 stabilizes B cell receptor signalosome assembly. Sci Signal 7, ra89.
45. Wang, Y., Sosinowski, T., Novikov, A., Crawford, F., Neau, D. B., Yang, J., Kwok, W. W., Marrack, P., Kappler, J. W., and Dai, S. (2018). C-terminal modification of the insulin B:11-23 peptide creates superagonists in mouse and human type 1 diabetes. Proceedings of the National Academy of Sciences of the United States of America 115, 162-167.
46. Wardemann, H., Yurasov, S., Schaefer, A., Young, J. W., Meffre, E., and Nussenzweig, M. C. (2003). Predominant autoantibody production by early human B cell precursors. Science 301, 1374-1377.
47. Watson, C. T., Steinberg, K. M., Huddleston, J., Warren, R. L., Malig, M., Schein, J., Willsey, A. J., Joy, J. B., Scott, J. K., Graves, T. A., et al. (2013). Complete haplotype sequence of the human immunoglobulin heavy-chain variable, diversity, and joining genes and characterization of allelic and copy-number variation. Am J Hum Genet 92, 530-546.
48. Wu, A. R., Neff, N. F., Kalisky, T., Dalerba, P., Treutlein, B., Rothenberg, M. E., Mburu, F. M., Mantalas, G. L., Sim, S., Clarke, M. F., et al. (2014). Quantitative assessment of single-cell RNA-sequencing methods. Nature methods 11, 41-46.
49. Xia, Z., Chen, H., Kang, S. G., Huynh, T., Fang, J. W., Lamothe, P. A., Walker, B. D., and Zhou, R. (2014). The complex and specific pMHC interactions with diverse HIV-1 TCR clonotypes reveal a structural basis for alterations in CTL function. Scientific reports 4, 4087.
50. Xiao, Z., Mohamood, A. S., Uddin, S., Gutfreund, R., Nakata, C., Marshall, A., Kimura, H., Caturegli, P., Womer, K. L., Huang, Y., et al. (2011). Inhibition of Fas ligand in NOD mice unmasks a protective role for IL-10 against insulitis development. Am J Pathol 179, 725-732.
51. Yoneshiro, T., Matsushita, M., Hibi, M., Tone, H., Takeshita, M., Yasunaga, K., Katsuragi, Y., Kameya, T., Sugie, H., and Saito, M. (2017). Tea catechin and caffeine activate brown adipose tissue and increase cold-induced thermogenic capacity in humans. Am J Clin Nutr 105, 873-881.
52. Yu, L. M., and Chang, T. W. (1992). Human mb-1 gene: complete cDNA sequence and its expression in B cells bearing membrane Ig of various isotypes. Journal of immunology 148, 633-637.
53. Ziegenhain, C., Vieth, B., Parekh, S., Reinius, B., Guillaumet-Adkins, A., Smets, M., Leonhardt, H., Heyn, H., Hellmann, I., and Enard, W. (2017). Comparative Analysis of Single-Cell RNA Sequencing Methods. Mol Cell 65, 631-643 e634.

### Example 2: Detection of x-mAb in Patient Samples

The present inventors developed ELISA assays to measure the presence of x-mAb in blood serum. In certain embodiments, the method can be used to screen for individuals bearing the x-idiotype to identify at-risk individuals and/or to confirm diagnosis of T1D in new patients. In particular embodiments, blood serum from healthy individuals is used as a control.

Protocol:

1. Coat high affinity 96 well plate with various concentrations of anti-x-mAb in bicarbonate buffer overnight at 40 C.

2. Wash plates four times with 200 ul of phosphate buffer saline with 0.05%

3. Block plates using blocking buffer (20% FCS) for 2 hr at 370 C.

4. Prepare two fold serial dilutions of serum in blocking buffer starting at 1:50.

5. Wash plates 6 times with 200 microliters of PBST.

6. Add diluted serum in three replicate wells.

7. Use wells that lack anti-x-mAb or serum as control for background signals.

8. Incubate plates overnight at 40 C.

9. Wash plates 6 times with 200 microliters of PBST.

10. Prepare the secondary antibody (goat anti-human IgG) diluted 1:10,000.

11. Add secondary antibody to different wells as indicated.

12. Incubate plates at 370 C for 30 min.

13. Wash plates with 200 microliters of PBST 6 times.

14. Add TMB solution to each well for 10 mins and then stop reaction using sulfuric acid.

15. Read the plate using ELISA read at 405 nm and 550 nm.

16. Calculate the averages and normalization with its 550 nm calculations.

17. Statistically significant signals are defined as p<0.05%.

### Example 3: Prediction of Type 1 Diabetes

A labeled x-mAb documents that the presence of T1D specific X-cells in individuals at high risk of developing T1D (for example, who have 2 or more T1D antibodies) predicts who will go on to develop T1D.

TrialNet collects and stores serial blood samples from individuals having 1 or more T1D antibodies, including those who do and do not go on to develop T1D. Frozen samples from TrailNet from individuals with one or more T1D antibodies are used to show that those with T1D specific X-cells predicts who goes on to develop T1D.

### Example 4: Prevention and Treatment of Type 1 Diabetes

Humanized antibodies to the T1D disease-specific amino acid sequence on the surface of the X-cell (“x-mAbs”) that bind to and either inactivate or destroy the cell prevent its pathogenic actions. When X-cells are added to a population of T1D autoreactive T cells, they become markedly activated, divide rapidly and secrete cytokines. Experiments are performed to demonstrate the neutralizing action of the x-mAb, experiments are performed, including:

1. x-mAbs and T1D specific X-cells are added to a population of T1D autoreactive T cells to demonstrate that cytokine release and expansion of autoreactive T cells is blocked in vitro.

2. In a humanized mouse model of T1D, the addition of T1D specific X-cells leads to lymphocytic infiltration of islet cells of the pancreas (insulitis) and T1D.

3. In a humanized mouse model of T1D, treating mice given T1D specific X-cells with x-mAbs prevents insulitis and T1D from developing.

4. Treating patients at high risk for developing T1D (e.g., those having 2 or more T1D antibodies) with x-mAbs prevents the disease.

5. Treating patients at high risk for developing T1D (e.g., having the T1D specific X-cells and 1 or more T1D antibodies) with x-mAbs prevents the disease.

1. A method comprising detecting a nucleotide sequence encoding SEQ ID NO:1 from a biological sample obtained from a patient.

2. The method of claim 1, wherein the detecting step comprises polymerase chain reaction (PCR).

3. The method of claim 1, wherein the PCR comprises amplification using primers comprising SEQ ID NOS:17-18.

4. The method of claim 1, wherein the PCR comprises amplification using primers comprising SEQ ID NOS:23 and 19.

5. The method of claim 1, wherein the nucleotide sequence comprises SEQ ID NO:25.

6. The method of claim 1, wherein the biological sample is peripheral blood.

7. The method of claim 1, wherein the detecting step further comprises sequencing.

8. The method of claim 1, further comprising the step of genotyping the HLA-DQ allele.

9. A method for determining whether a patient is at-risk for Type 1 Diabetes (T1D) comprising the steps of:
(a) detecting a nucleotide sequence encoding SEQ ID NO:1 from a biological sample obtained from a patient;
(b) genotyping HLA-DQ to detect the presence of the HLA-DQ7 allele or the HLA-DQ8 allele,
wherein a patient having SEQ ID NO:1 and HLA-DQ8 is at risk for T1D, a patient not having SEQ ID NO:1 and HLA-DQ7 is not at risk for T1D, and a patient not having SEQ ID NO:1 is not at risk for T1D.

10. A method for determining whether a patient is at-risk for an autoimmune disease comprising the steps of:
(c) detecting a nucleotide sequence encoding SEQ ID NO:1 from a biological sample obtained from a patient;
(d) genotyping the HLA-DQ to detect the presence of the HLA-DQ7 allele or the HLA-DQ8 allele,
wherein a patient having SEQ ID NO:1 and HLA-DQ8 is at risk for an autoimmune disease, a patient not having SEQ ID NO:1 and HLA-DQ7 is not at risk for an autoimmune disease, and a patient not having SEQ ID NO:1 is not at risk for an autoimmune disease.

11. The method of claim 10, wherein the autoimmune disease comprises rheumatoid arthritis, multiple sclerosis, or systemic lupus erythematosus.

12. An antibody or antigen-binding fragment thereof that specifically binds SEQ ID NO:1.

13. An antibody or antigen-binding fragment thereof that specifically binds (i) a B-cell receptor expressed on a lymphocyte, wherein the B-cell receptor comprises SEQ ID NO:1; or (ii) a free-floating antibody comprising SEQ ID NO:1.

14. An antibody or antigen-binding fragment thereof that specifically binds an antibody comprising SEQ ID NO:1.

15. The antibody or antigen-binding fragment of claim 13, wherein the antibody or antigen-binding fragment prevents or reduces the binding of antigen to SEQ ID NO:1.

16. The antibody or antigen-binding fragment of claim 13, wherein the antigen-binding fragment is selected from the group consisting of an scFv, sc(Fv)2, Fab, F(ab)2, and a diabody.

17. A method for treating or preventing type 1 diabetes (T1D) in a subject having T1D or a risk thereof comprising the step of administering to the patient a therapeutically effective amount of the antibody or antigen-binding fragment of claim 13.

18. An isolated antibody or antibody-binding fragment thereof comprising heavy chain complementarity determining regions (CDRs) 1, 2 and 3, wherein the heavy chain CDR1 comprises an amino acid sequence as set forth in SEQ ID NO:60, or the amino acid sequence as set forth in SEQ ID NO:60 with a substitution at two or fewer amino acid positions, the heavy chain CDR2 comprising an amino acid set forth in SEQ ID NO:62, or the amino acid set forth in SEQ ID NO:62 with a substitution at two or fewer amino acid positions, and the heavy chain CDR3 comprising an amino acid sequence as set forth in SEQ ID NO:64, or the amino acid sequence as set forth in SEQ ID NO:64 with a substitution at two or fewer amino acid positions.

19. The isolated antibody of claim 18, wherein the isolated antibody or antigen-binding fragment further comprises light chain CDRs 1, 2 and 3, wherein the light chain CDR1 comprises an amino acid sequence as set forth in SEQ ID NO:66, or the amino acid sequence as set forth in SEQ ID NO:66 with a substitution at two or fewer amino acid positions, the light chain CDR2 comprising an amino acid sequence as set forth in SEQ ID NO:68, or the amino acid sequence as set forth in SEQ ID NO:68 with a substitution at two or fewer amino acid positions, and the light chain CDR3 comprising an amino acid sequence as set forth in SEQ ID NO:9, or the amino acid sequence as set forth in SEQ ID NO:9 with a substitution at two or fewer amino acid positions.

20. An isolated antibody or antigen-binding fragment thereof that specifically binds (1) a B-cell receptor expressed on a lymphocyte, wherein the B-cell receptor comprises SEQ ID NO:1, or (2) a free-floating antibody comprising SEQ ID NO:1, wherein the antibody or antigen-binding fragment thereof comprises a heavy chain variable region (VH) comprising CDR1, CDR2, and CDR3 consisting of the amino acid sequences as set forth in SEQ ID NOS:60, 62 and 64, respectively.

21. An isolated antibody or antigen-binding fragment thereof that specifically binds (1) a B-cell receptor expressed on a lymphocyte, wherein the B-cell receptor comprises SEQ ID NO:1, or (2) a free-floating antibody comprising SEQ ID NO:1, wherein the antibody or antigen-binding fragment thereof comprises a light chain variable region (VL) comprising CDR1, CDR2, and CDR3 consisting of the amino acid sequences as set forth in SEQ ID NOS: 66, 68 and 9, respectively.

22. An isolated antibody or antigen-binding fragment thereof that specifically binds (1) a B-cell receptor expressed on a lymphocyte, wherein the B-cell receptor comprises SEQ ID NO:1, or (2) a free-floating antibody comprising SEQ ID NO:1, wherein the antibody or antigen-binding fragment thereof comprises:
(a) a VH comprising CDR1, CDR2, and CDR, consisting of the amino acid sequences as set forth in SEQ ID NOS:60, 62 and 64, respectively; and
(b) a VL comprising CDR1, CDR2, and CDR3, consisting of the amino acid sequences as set forth in SEQ ID NOS:66, 68 and 9.

23. An isolated nucleic acid molecule encoding the antibody or antigen-binding fragment thereof of claim 22.

24. A vector comprising a nucleic acid molecule of claim 23.

25. A host cell comprising a vector of claim 24.

26. The host cell of claim 25, wherein the host cell is a prokaryotic or a eukaryotic cell.

27. A method for producing an antibody or antigen-binding fragment thereof comprising the steps of (a) culturing a host cell of claim 25 under conditions suitable for expression of the antibody or antigen-binding fragment thereof by the host cells; and (b) recovering the antibody or antigen-binding fragment thereof.

28. The method of claim 27, wherein the host cell is a prokaryotic or a eukaryotic cell.

29. A composition comprising the antibody or antigen-binding fragment thereof according to claim 22 and a suitable pharmaceutical carrier.

30. The composition of claim 29, wherein the composition is formulated for intravenous, intramuscular, oral, subcutaneous, intraperitoneal, intrathecal or intramuscular administration.

31. A method of treating diabetes in a mammal comprising the step of administering to the mammal a therapeutically effective amount of the antibody or antigen-binding fragment thereof of claim 22.

32. A method of treating an autoimmune disease in a mammal comprising the step of administering to the mammal a therapeutically effective amount of the antibody or antigen-binding fragment thereof of claim 22.

33. The antibody or antigen-binding fragment of claim 18, wherein the antigen-binding fragment is selected from the group consisting of an scFv, sc(Fv)2, Fab, F(ab)2, and a diabody.

34. An isolated antibody or antigen-binding fragment thereof that specifically binds (1) a B-cell receptor expressed on a lymphocyte, wherein the B-cell receptor comprises SEQ ID NO:1, or (2) a free-floating antibody comprising SEQ ID NO:1, wherein the antibody or antigen-binding fragment thereof comprises a VH comprising the amino acid sequence set forth in SEQ ID NO:74.

35. An isolated antibody or antigen-binding fragment thereof that specifically binds (1) a B-cell receptor expressed on a lymphocyte, wherein the B-cell receptor comprises SEQ ID NO:1, or (2) a free-floating antibody comprising SEQ ID NO:1, wherein the antibody or antigen-binding fragment thereof comprises a VL comprising the amino acid sequence as set forth in SEQ ID NO:76.

36. An isolated antibody or antigen-binding fragment thereof that specifically binds (1) a B-cell receptor expressed on a lymphocyte, wherein the B-cell receptor comprises SEQ ID NO:1, or (2) a free-floating antibody comprising SEQ ID NO:1, wherein the antibody or antigen-binding fragment thereof comprises a VH comprising the amino acid sequence as set forth in SEQ ID NO:74 and a VL comprising the amino acid sequence as set forth in SEQ ID NO:76.

37. An isolated antibody or antibody-binding fragment thereof comprising heavy chain complementarity determining regions (CDRs) 1, 2 and 3, wherein the heavy chain CDR1 comprises an amino acid sequence as set forth in SEQ ID NO:78, or the amino acid sequence as set forth in SEQ ID NO:78 with a substitution at two or fewer amino acid positions, the heavy chain CDR2 comprising an amino acid set forth in SEQ ID NO:80, or the amino acid set forth in SEQ ID NO:80 with a substitution at two or fewer amino acid positions, and the heavy chain CDR3 comprising an amino acid sequence as set forth in SEQ ID NO:82, or the amino acid sequence as set forth in SEQ ID NO:82 with a substitution at two or fewer amino acid positions.

38. The isolated antibody of claim 37, wherein the isolated antibody or antigen-binding fragment further comprises light chain CDRs 1, 2 and 3, wherein the light chain CDR1 comprises an amino acid sequence as set forth in SEQ ID NO:84, or the amino acid sequence as set forth in SEQ ID NO:84 with a substitution at two or fewer amino acid positions, the light chain CDR2 comprising an amino acid sequence as set forth in SEQ ID NO:86, or the amino acid sequence as set forth in SEQ ID NO:86 with a substitution at two or fewer amino acid positions, and the light chain CDR3 comprising an amino acid sequence as set forth in SEQ ID NO:88, or the amino acid sequence as set forth in SEQ ID NO:88 with a substitution at two or fewer amino acid positions.

39. An isolated antibody or antigen-binding fragment thereof that specifically binds (1) a B-cell receptor expressed on a lymphocyte, wherein the B-cell receptor comprises SEQ ID NO:1, or (2) a free-floating antibody comprising SEQ ID NO:1, wherein the antibody or antigen-binding fragment thereof comprises a heavy chain variable region (VH) comprising CDR1, CDR2, and CDR3 consisting of the amino acid sequences as set forth in SEQ ID NOS:78, 80 and 82, respectively.

40. An isolated antibody or antigen-binding fragment thereof that specifically binds (1) a B-cell receptor expressed on a lymphocyte, wherein the B-cell receptor comprises SEQ ID NO:1, or (2) a free-floating antibody comprising SEQ ID NO:1, wherein the antibody or antigen-binding fragment thereof comprises a light chain variable region (VL) comprising CDR1, CDR2, and CDR3 consisting of the amino acid sequences as set forth in SEQ ID NOS: 84, 86 and 88, respectively.

41. An isolated antibody or antigen-binding fragment thereof that specifically binds (1) a B-cell receptor expressed on a lymphocyte, wherein the B-cell receptor comprises SEQ ID NO:1, or (2) a free-floating antibody comprising SEQ ID NO:1, wherein the antibody or antigen-binding fragment thereof comprises:
(a) a VH comprising CDR1, CDR2, and CDR, consisting of the amino acid sequences as set forth in SEQ ID NOS:78, 80 and 82, respectively; and
(b) a VL comprising CDR1, CDR2, and CDR3, consisting of the amino acid sequences as set forth in SEQ ID NOS:84, 86 and 88.

42. An isolated nucleic acid molecule encoding the antibody or antigen-binding fragment thereof of claim 41.

43. A vector comprising a nucleic acid molecule of claim 42.

44. A host cell comprising a vector of claim 43.

45. The host cell of claim 44, wherein the host cell is a prokaryotic or a eukaryotic cell.

46. A method for producing an antibody or antigen-binding fragment thereof comprising the steps of (a) culturing a host cell of claim 44 under conditions suitable for expression of the antibody or antigen-binding fragment thereof by the host cells; and (b) recovering the antibody or antigen-binding fragment thereof.

47. The method of claim 46, wherein the host cell is a prokaryotic or a eukaryotic cell.

48. A composition comprising the antibody or antigen-binding fragment thereof according to claim 41 and a suitable pharmaceutical carrier.

49. The composition of claim 48, wherein the composition is formulated for intravenous, intramuscular, oral, subcutaneous, intraperitoneal, intrathecal or intramuscular administration.

50. A method of treating diabetes in a mammal comprising the step of administering to the mammal a therapeutically effective amount of the antibody or antigen-binding fragment thereof of claim 41.

51. A method of treating an autoimmune disease in a mammal comprising the step of administering to the mammal a therapeutically effective amount of the antibody or antigen-binding fragment thereof of claim 41.

52. The antibody or antigen-binding fragment of claim 41, wherein the antigen-binding fragment is selected from the group consisting of an scFv, sc(Fv)2, Fab, F(ab)2, and a diabody.

53. An isolated antibody or antigen-binding fragment thereof that specifically binds (1) a B-cell receptor expressed on a lymphocyte, wherein the B-cell receptor comprises SEQ ID NO:1, or (2) a free-floating antibody comprising SEQ ID NO:1, wherein the antibody or antigen-binding fragment thereof comprises a VH comprising the amino acid sequence set forth in SEQ ID NO:90.

54. An isolated antibody or antigen-binding fragment thereof that specifically binds (1) a B-cell receptor expressed on a lymphocyte, wherein the B-cell receptor comprises SEQ ID NO:1, or (2) a free-floating antibody comprising SEQ ID NO:1, wherein the antibody or antigen-binding fragment thereof comprises a VL comprising the amino acid sequence as set forth in SEQ ID NO:92.

55. An isolated antibody or antigen-binding fragment thereof that specifically binds (1) a B-cell receptor expressed on a lymphocyte, wherein the B-cell receptor comprises SEQ ID NO:1, or (2) a free-floating antibody comprising SEQ ID NO:1, wherein the antibody or antigen-binding fragment thereof comprises a VH comprising the amino acid sequence as set forth in SEQ ID NO:90 and a VL comprising the amino acid sequence as set forth in SEQ ID NO:92.
